(19) United States
(12) Patent Application Publication
(10) Pub. No.: US 2005/0049146 A1
(43) Pub. Date: Mar. 3, 2005

(54) Use of substituted dibenzothiepine derivatives as insecticidal, acaricidal and nematicidal agents

(76) Inventors: John F. Chiarello, Newtown, PA (US); William Wakefield Wood, Pennington, NJ (US); Deborah Culbertson, Wycombe, PA (US); Daniel W. Smith, Skillman, NJ (US); Robin C. Sperry, Burlington Twp, NJ (US); Thomas Schmidt, Neustadt (DE); Gerd Steiner, Kirchheim (DE); Markus Kordes, Frankenthal (DE); Wolfgang von Deyn, Neustadt (DE); Norbert Gotz, Worms (DE); Michael Hofmann, Ludwigshafen (DE); Keith Barnes, Rexford, NY (US); James Takasugi, Lawrenceville, NJ (US); Michael F. Treacy, Newton, PA (US); Hassan Oloumi-sadeghi, Raleigh, NC (US); Robert Diehl, Sunset Beach, NC (US)

Correspondence Address: KEIL & WEINKAUF, 1350 CONNECTICUT AVENUE, N.W., WASHINGTON, DC, 20036, US

(21) Appl. No.: 10/494,770

(22) PCT Filed: Nov. 2, 2002
(86) PCT No.: PCT/EP02/12228

Related U.S. Application Data
(60) Provisional application No. 60/337,463, filed on Nov. 5, 2001.

(51) Int. Cl. A01N 43/46; A01N 43/02; C07D 223/18

(52) U.S. Cl. 504/218; 504/288; 504/291; 540/588; 549/12; 549/354

(57) ABSTRACT

A composition comprising a) compounds of formula (I) 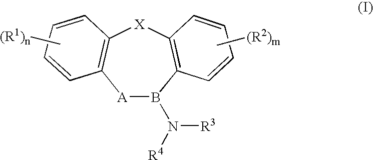 wherein A-B denotes C—C or C═C;
X is S, O, SO, SO₂, NR^{a}, or CR^{b}R^{c};
R¹, R² are H, halogen, OH, SH, NH₂, CN, NO₂, alkyl, alkoxy, alkylamino, dialkylamino, alkylthio, alkenyl, alkenyloxy, alkenylamino, alkenylthio, alkynyl, alkynyloxy, alkynylamino, alkynylthio, alkylsulfonyl, alkylsulfoxyl, alkenylsulfonyl, alkynylsulfoxyl, formyl, alkylcarbonyl, hydroxycarbonyl, alkoxycarbonyl, carbonyloxy, alkylcarbonyloxy, phenyloxy, C(O)NR^{d}R^{e}, (SO₂)ₙNR^{d}R^{e}, or C(═NOR^{f})-Γₚ-R^{f}′, a mono- or bicyclic 5- to 10-membered aromatic ringsystem, a 5- to 10-membered heteroaromatic ring system, or cycloalkyl, each optionally substituted, which is unfused or fused to the aromatic group to which it is bonded and which, when unfused, is bonded directly or through an O, S, alkyl, or alkoxy linkage;
R³, R⁴ are each independently H, alkyl, haloalkyl, alkylamine, alkoxy, cycloalkyl, wherein the carbon atoms in these aliphatic groups may be substituted,
   or R³ and R⁴ together with the nitrogen atom to which they are attached form a saturated or partially saturated mono-or bi-cyclic 5- to 10-membered ringsystem or a 5-membered hetaryl, wherein the rings are optionally substituted and are bonded directly or via an O, S, or alkyl linkage, and cycloalkenyl,
   wherein the carbon atoms in these aliphatic groups can be substituted, and wherein the group NR³R⁴ can be present as [N⁺—O⁻]R³R⁴ (aminoxide) if R³ and R⁴ are bonded through a single bond to the nitrogen atom;
   and R^{a}, R^{b}, R^{c}, R^{d}, R^{e}, R^{f}′, R^{f}, Γ and p are as defined in the description; m is 0, 1, 2, 3, or 4; n is 0, 1, 2, 3, or 4; or the enantiomers or diastereomers, salts or esters thereof, and b) an agronomically acceptable carrier, methods for protecting crops from attack by insects, arachnids and nematodes by contacting the crop or the target species with a pesticidally effective amount of the compound of formula (I), compounds of formula I, and a process for their preparation.

The present invention relates to pesticidal compositions that include a) compounds of formula (I): 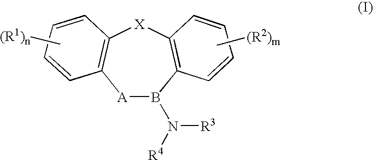
wherein
A-B denotes C—C or C═C;
X is sulfur, oxygen, sulfinyl (S═O), sulfonyl (SO₂), NR^{a}, or CR^{b}R^{c};
   R^{a} hydrogen, C₁-C₆-alkyl, C₂-C₆-alkenyl, or C₂-C₆-alkynyl wherein the carbon atoms in these groups may be substituted by 1 to 3 groups R^{#}
      R^{#} halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, or C₁-C₆-alkylthio;
      phenyl or benzyl, each unsubstituted or substituted with any combination of 1 to 5 halogen, 1 to 3 C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
   R^{b}, R^{c} are each independently hydrogen, hydroxy, amino, C₁-C₆-alkyl, C₂-C₆-alkenyl, C₂-C₆-alkinyl, C₁-C₆-hydroxyalkyl, C₁-C₆-alkylamino, di(C₁-C₆-alkyl)amino, wherein the carbon atoms in these groups may be substituted by 1 to 3 groups R^{#} or
      phenyl, unsubstituted or substituted with any combination of 1 to 5 halogen, 1 to 3 C₁-C₆-alkyl, C₁-C₆-haloalkyl, C₁-C₆-alkoxy or C₁-C₆-haloalkoxy groups, or
      CR^{b}R^{c} represents C═O or C═CR^{j}R^{k}, wherein R^{j} and R^{k} each independently are hydrogen, halogen, cyano, C₁-C₆-alkyl, C₁-C₆-haloalkyl, C₃-C₆-cycloalkyl;
R¹, R² are each independently hydrogen, halogen, hydroxy, mercapto, amino, cyano, nitro,
   C₁-C₆-alkyl, C₁-C₆-alkoxy, C₁-C₆-alkylamino, di(C₁-C₆-alkyl)amino, C₁-C₆-alkylthio, C₂-C₆-alkenyl, C₂-C₆-alkenyloxy, C₂-C₆-alkenylamino, C₂-C₆-alkenylthio, C₂-C₆-alkynyl, C₂-C₆-alkynyloxy, C₂-C₆-alkynylamino, C₂-C₆-alkynylthio, C₁-C₆-alkylsulfonyl, C₁-C₆-alkylsulfoxyl, C₂-C₆-alkenylsulfonyl, C₂-C₆-alkynylsulfoxyl, formyl, C₁-C₆-alkylcarbonyl, hydroxycarbonyl, C₁-C₆-alkoxycarbonyl, carbonyloxy, C₁-C₆-alkylcarbonyloxy, phenyloxy, C(O)NR^{d}R^{e}, (SO₂)ₙNR^{d}R^{e}, wherein the carbon atoms in the aliphatic and aromatic groups may be substituted by 1 to 3 groups R^{#} wherein R^{d} and R^{e} are each independently groups as listed for R^{a}; or
   C(═NOR^{f})-Γₚ-R^{f}′, wherein R^{f}′ and R^{f} are each independently hydrogen or C₁-C₆-alkyl, F is oxygen, sulfur or NR^{f} and p is 0 or 1; or
   a mono- or bicyclic 5- to 10-membered aromatic ringsystem which may contain 1 to 4 heteroatoms selected from oxygen, sulfur and nitrogen and which is unfused or fused to the aromatic group to which it is bonded and which, when unfused, is bonded directly or through an oxygen, sulfur, C₁-C₆-alkyl, or C₁-C₆-alkoxy linkage, and which is unsubstituted or substituted with any combination of 1 to 5 groups R^{#}; or
   C₃-C₁₂-cycloalkyl, which is bonded directly or through an oxygen, sulfur or C₁-C₆-alkyl linkage, and which is unsubstituted or substituted with any combination of 1 to 5 groups R^{#};
R³, R⁴ are each independently hydrogen, C₁-C₆-alkyl, C₁-C₆-haloalkyl, C₁-C₆-alkylamino, C₁-C₆-alkoxy, C₃-C₆-cycloalkyl, wherein the carbon atoms in these aliphatic groups may be substituted by 1 to 3 groups R^{#}, or C(O)R^{g}, C(O)NR^{h}Rⁱ C(S)NR^{h}Rⁱ,
   R^{g} hydrogen, C₁-C₆-alkyl, C₁-C₆-alkoxy, or
      phenyl or benzyl, each unsubstituted or substituted with any combination of 1 to 5 halogen, 1 to 3 C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
   R^{h}, Rⁱ are each independently groups as listed for R^{a};
   or R³ and R⁴ together with the nitrogen atom to which they are attached form a saturated or partially saturated mono- or bi-cyclic 5- to 10-membered ringsystem containing 1 to 3 heteroatoms selected from nitrogen and oxygen or
   5-membered hetaryl containing 1 to 4 nitrogen atoms, wherein the carbon and/or nitrogen atoms in the saturated, partially saturated or aromatic rings are unsubstituted or substituted with any combination of 1 to 4 groups selected from amino, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-alkylamino, di(C₁-C₆-alkyl)amino, C₂-C₆-alkenylamino, C₂-C₆-alkynylamino, C₁-C₆-hydroxyalkyl, hydroxycarbonyl-C₁-C₄-alkyl, C₁-C₆-alkoxycarbonyl -C₁-C₄-alkyl, formyl-C₁-C₄-alkyl, formyl-C₁-C₄-alkoxy, C₁-C₆-alkylcarbonyl-C₁-C₄-alkoxy, C₃-C₆-cycloalkyl, which is bonded directly or via an oxygen, sulfur or C₁-C₆-alkyl linkage, and C₅-C₈-cycloalkenyl, wherein the carbon atoms in these aliphatic groups can be substituted by 1 to 4 groups selected from halogen, cyano, hydroxy and nitro,
   and wherein the nitrogen atoms which form part of the saturated rings can be present as [N⁺—O⁻] (aminoxide);
m is 0, 1, 2, 3 or 4;
n is 0, 1, 2, 3 or 4;
   or the enantiomers or diastereomers, salts or esters thereof, and b) an agronomically acceptable carrier.

In spite of the commercial insecticides, acaricides and nematicides available today, damage to crops, both growing and harvested, caused by insects and nematodes still occurs. Therefor, there is continuing need to develop new and more effective insecticidal, acaricidal and nematicidal agents.

It was therefore an object of the present invention to provide new pesticidal compositions, new compounds and new methods for the control of insects, arachnids or nematodes and of protecting growing plants from attack or infestation by insects, arachnids or nematodes.

We have found that these objects are achieved by the compositions and the compounds of formula I. Furthermore, we have found processes for preparing the compounds of formula I.

Some compounds of formula I have been described inter alia in U.S. Pat. No. 3,351,599 and Pharm. Ind. 32, p. 923-935 (1970). However, an insecticidal, acaricidal or nematicidal activity of compounds of formula I has not been known yet.

The present invention provides a method for the control of insects, arachnids or nematodes by contacting an insect, arachnid or nematode or their food supply, habitat or breeding grounds with a pesticidally effective amount of compounds of formula I.

Moreover, the present invention also relates to a method of protecting growing plants from attack or infestation by insects, arachnids or nematodes by applying to the foliage of the plants, or to the soil or water in which they are growing, a pesticidally effective amount of compounds of formula I.

Depending on the substitution pattern, the compounds of formula I can contain one or more chiral centers, in which case they are present as enantiomer or diastereomer mixtures. Subject-matter of this invention are not only compositions containing these mixtures but also those containing the pure enantiomers or diastereomers.

The compounds useful in the present invention may be readily synthesized using techniques generally known by synthetic organic chemists. Exemplary synthesis methods are taught in U.S. Pat. No. 3,351,599, which is incorporated by reference.

Compounds of formula I wherein X is oxygen are for example obtainable according to the procedure described in Chem. Pharm. Bull. 23, pp 2223-2231 (1975).

Compounds of formula I wherein X is methylene are for example obtainable according to the procedure described in U.S. Pat. No. 3,496,182.

Compounds of formula I wherein X is NR^{a} are for example obtainable following the procedures described in J. Med. Chem. 14, p. 56-58 (1971), and J. Med. Chem. 13, p. 979-981 (1970).

Compounds of formula I-2A for example wherein R^{z} is hydrogen, amino, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-alkylamino, di(C₁-C₆-alkyl)amino, C₂-C₆-alkenylamino, C₂-C₆-alkynylamine, C₁-C₆-hydroxyalkyl, hydroxycarbonyl-C₁-C₄-alkyl, C₁-C₆-alkoxycarbonyl-C₁-C₄-alkyl, formyl-C₁-C₄-alkyl, formyl-C₁-C₄-alkoxy, C₁-C₆-alkylcarbonyl-C₁-C₄-alkoxy, C₃-C₆-cycloalkyl, which is bonded directly or through an oxygen, sulfur or C₁-C₆-alkyl linkage, or C₅-C₈-cycloalkenyl, wherein the carbon atoms in these aliphatic groups can be substituted by 1 to 4 groups selected from halogen, cyano, hydroxy and nitro, o is 1 or 2, and the further variables and the indices are as defined for formula I are preferably obtainable by a five step reaction, 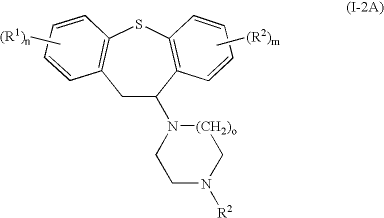
wherein in a first step benzoic acid derivates of formula II are reacted with thiophenol derivates III in the presence of a base to give compounds IV, wherein the variables and the indices of the compounds II, III and IV have the meanings as defined for formula I.

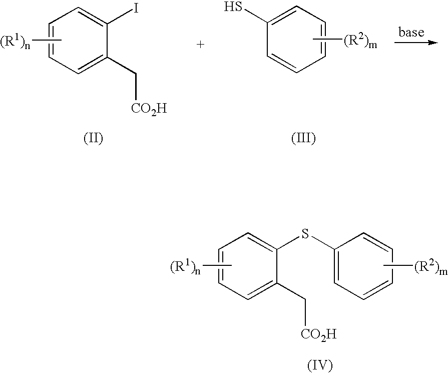

The reaction is usually carried out at temperatures of from −70° C. to 250° C., preferably from 25° C. to 150° C., in an inert organic solvent such as toluene or water in the presence of a base such as alkali metal and alkaline earth metal hydroxides, for example lithium hydroxide, sodium hydroxide, potassium hydroxide, calcium hydroxide, and potassium carbonate, and preferably a transition metal halogenid such as CuCl, CuBr or CuI as a catalyst [lit. Houben-Weyl, Methoden der organischen Synthese, 4^{th} edition, Bd. 9, p. 112-113, Georg Thieme Verlag Stuttgart].

Compounds of formula II are obtainable by customary methods, or are commercially available [see e.g. J. March, Advanced Organic Chemistry, 4th edition, chapters 11, 13, 14, and 15, John Wiley & Sons New York].

Compounds of formula III are commercially available.

In a second step, compounds of formula IV are transformed into compounds of formula V, wherein the variables and the indices have the meanings as defined for formula I, by cyclization with polyphosphoric acid [lit.: U.S. Pat. No. 3,351,599; Houben-Weyl, Methoden der organischen Synthese, 4^{th} edition, Bd. 7/2a, p. 15-22, Georg Thieme Verlag Stuttgart].

The reaction can optionally be carried out as a two-step process, preferably without isolation of the intermediates. Here, the carbon acid function of compounds IV first is activated, such as by converting carboxylic acids of formula V into their corresponding carboxylic acid halides, e.g. with chlorinating agent such as SOCl₂, POCl₃, PCl₅, or (COCl)₂ [lit.: J. March, Advanced Organic Chemistry: reactions, mechanisms and structure, 4th ed. 1992, Wiley&Sons, New York]. The thiepine ring then is formed via a Friedl-Crafts acylation with a Lewis acid such as AlCl₃, AlBr₃, FeCl₃, ZnCl₂, SnCl₄, or TiCl₄.

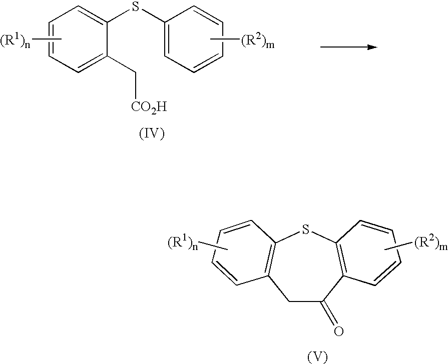

The reaction is usually carried out at temperatures of from −70° C. to 250° C., preferably from 0° C. to 100° C., in an inert organic solvent [lit.: Houben-Weyl, Methoden der organischen Synthese, 4th edition, Bd. 7/2a, p. 15-22, Georg Thieme Verlag Stuttgart].

In a third step, compounds of formula V are hydrogenated to give compounds of formula VI, wherein the variables and the indices have the meanings as defined for formula I, and the hydrogenating agent for example is a metal hydride or metal boron hyride such as NaBH₄ or LiAlH₄.

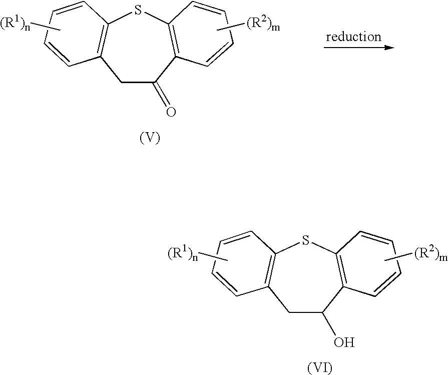

The reaction is usually carried out at temperatures of from −70° C. to 250° C., preferably from 0° C. to 100° C., in an inert organic solvent such as alcohols, for example ethanol [lit.: U.S. Pat. No. 3,351,599].

In a forth step, compounds of formula VI are halogenated to give compounds of formula VII, wherein the variables and the indices have the meanings as defined for formula I. The halogenating agent may be a chlorinating agent such as SOCl₂ or a brominating agent such as HBr.

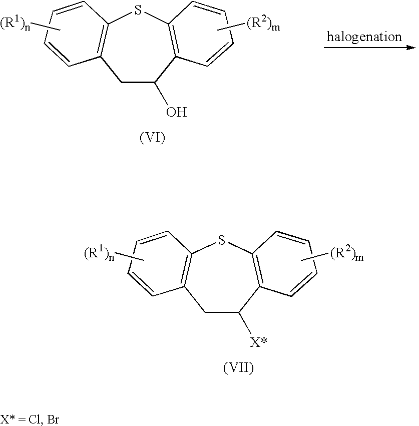

The reaction is usually carried out at temperatures of from −70° C. to 250° C., preferably from −10° C. to 100° C., in an inert organic solvent, optionally in the pure chlorinating agent (SOCl₂) [lit.: U.S. Pat. No. 3,351,599].

In a last step, compounds of formula VII are reacted with piperazin derivates VIII, wherein o and R^{z} are as defined for formula I-2A, to give compounds I-2A.

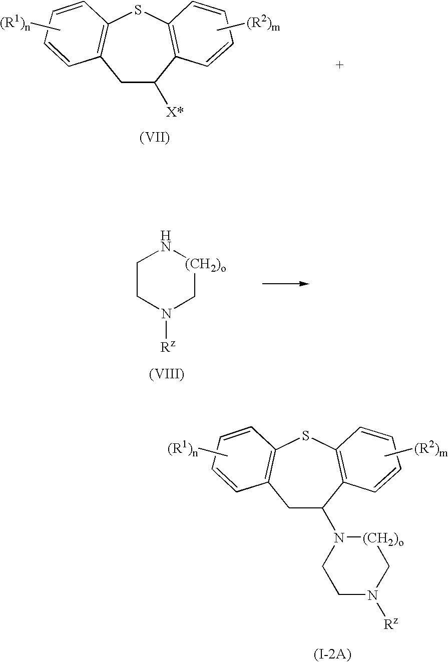

The reaction is usually carried out at temperatures of from −70° C. to 250° C., preferably from 0° C. to 200° C., in an inert organic solvent or pure compound VIII [lit. U.S. Pat. No. 3,351,599].

Compounds of formula VIII are commercially available or can be prepared following methods described in the literature [see e.g. J. Org. Chem. 31, p. 3867-3868 (1966)].

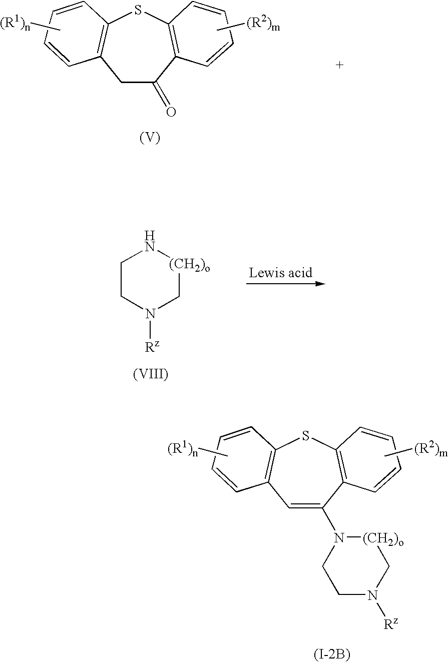

Compounds of formula I-2B wherein the variables and the indices have the meanings as defined for formula I-2A can be prepared by reacting compounds of formula V with piperazines VIII in the presence of an inert organic solvent and a Lewis acid such as AlCl₃, FeCl₃, ZnCl₂, SnCl₄, or TiCl₄.

The reaction is usually carried out at temperatures of from −70° C. to 250° C., preferably from −10° C. to 150° C., in an inert organic solvent in the presence of a Lewis acid [lit: Chem. Pharm. Bull. 23, p.2223-2231 (1975)].

Compounds of formula I-2A can be prepared from compounds of formula I-2B via reduction.

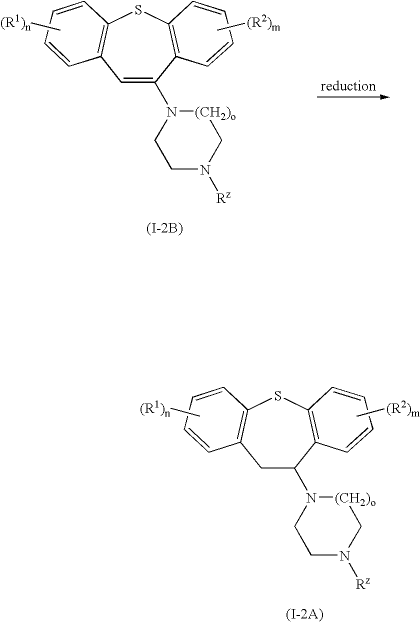

The reaction is usually carried out at temperatures of from −70° C. to 250° C., preferably from −20° C. to 100° C., in an inert organic solvent in the presence of an acid and a reduction agent such as metal boron hydrides such as LiBH₄ or diboranes, preferably NaBH₃CN [lit.: Houben-Weyl, Methoden der organischen Synthese, 4th edition, Bd. 4/1d, p. 76-78, Georg Thieme Verlag Stuttgart].

Suitable solvents are ethers, such as diethylether, diisopropylether, tert.-butylmethylether, digylme, dioxane, anisol and tetrahydrofuran, nitriles, alcoholes such as methanol, ethanol, n-propanol, isopropanol, n-butanol and tert.-butanol, and also dimethyl sulfoxide, dimethyl formamide and dimethyl acetamide. Preferred solvents are methanol and tetrahydrofuran. It is also possible to use mixtures of the solvents mentioned.

Suitable acids are inorganic acids such as hydrochloric acid, hydrobromic acid and sulfuric acid, and also organic acids, such as formic acid, acetic acid, propionic acid, oxalic acid, toluene sulfonic acid, benzene sulfonic acid, campher sulfonic acid, citric acid, and trifluoroacetic acid, preferably acetic acid. In general, the acid is employed to adjust a pH of 5.

The starting materials are generally reacted with one another in equimolar amounts. In terms of yield, it may be advantageous to use an excess of compounds of NaBH₃CN based on compounds I-2B.

The reaction mixtures are worked up in a customary manner, for example by mixing with water, phase separation and, if appropriate, chromatographic purification of the crude products. In some cases, the intermediates and end products are obtained in the form of colorless or pale brown viscous oils, which are purified or freed from volatile components under reduced pressure and at moderately elevated temperature. If the intermediates and end products are obtained as solids, they can also be purified by recrystallization or digestion.

If individual compounds I are not obtainable by the route described above, they can be prepared by derivatization of other compounds I or by customary modifications of the synthesis routes described.

The preparation of the compounds of formula I may lead to them being obtained as isomer mixtures. If desired, these can be resolved by the methods customary for this purpose, such as crystallization or chromatography, also on optically active adsorbate, to give the pure isomers.

Agronomically acceptable salts of the compounds I can be formed in a customary manner, e.g. by reaction with an acid of the anion in question.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydrocarbon group having 1 to 6 carbon atoms, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl groups having 1 to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

“Alkylamino” refers to a straight-chain or branched alkyl group having 1 to 6 carbon atoms (as mentioned above) which is bonded through a nitrogen linkage.

Similarly, “alkoxy” and “alkylthio” refer to straight-chain or branched alkyl groups having 1 to 6 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

The term “alkenyl” as used herein intends a branched or unbranched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

The term “alkynyl” as used herein refers to a branched or unbranched unsaturated hydrocarbon group containing at least one triple bond, such as ethynyl, propynyl, 1-butynyl, 2-butynyl, and the like.

Aryl: mono- or bicyclic 5- to 10-membered aromatic ringsystem, e.g. phenyl or naphthyl;

Hetaryl: a 5- to 10-membered heteroaromatic ring system containing 1 to 4 heteroatoms selected from oxygen, sulfur and nitrogen, e.g. 5-membered hetaryl, containing 1 to 4 nitrogen atoms, such as pyrrolyl, pyrazolyl, imidazolyl, triazolyl, and tetrazolyl; or
5-membered hetaryl, containing 1 to 4 nitrogen atoms or 1 to 3 nitrogen atoms and 1 sulfur or oxygen atom, e.g. furyl, thienyl, pyrrolyl, isoxazolyl, isothiazolyl, pyrazolyl, oxazolyl, thiazolyl, imidazolyl, oxadiazolyl, thiadiazolyl, oxadiazolyl, triazolyl, and tetrazolyl; or
5-membered hetaryl, containing 1 to 4 nitrogen atoms or 1 to 3 nitrogen atoms and 1 sulfur or oxygen atom, in which two adjacent ring carbon atoms or one nitrogen atom and an adjacent carbon atom can be bridged by buta-1,3-dien-1,4-diyl; or
6-membered hetaryl, containing 1 to 4 nitrogen atoms or 1 to 3 nitrogen atoms and 1 sulfur or oxygen atom, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

A saturated or partially saturated mono- or bicyclic 5- to 10-membered ringsystem containing 1 to 3 heteroatoms selected from nitrogen and oxygen intends e.g. a saturated monocyclic 5- to 7-membered ringsystem containing 1 to 3 heteroatoms selected from nitrogen and oxygen, such as pyridine, pyrimidine, pyrrolidine, piperazine, homopiperazine, morpholine, and piperidine; or e.g. a saturated bicyclic 7- to 10-membered ringsystem containing 1 to 3 heteroatoms selected from nitrogen and oxygen, such as 1,4-diazabicyclo[4.3.0]nonane, 2,5-diazabicyclo[2.2.2]octane, and 2,5-diazabicyclo[2.2.1]heptane.

Cycloalkyl: monocyclic 3- to 6-, 8-, 10- or 12-membered saturated carbon atom rings, e.g. C₃-C₈-cycloalkyl such as cyclopropyl, cyclobutyl, cyclopentyl, cyclohexyl, cycloheptyl and cyclooctyl.

With respect to the intended use of the compounds of formula I, particular preference is given to the following meanings of the substituents, in each case on their own or in combination:

Preference is given to compounds of formula I wherein A-B denotes C—C.

Moreover, preference is given to compounds of formula I wherein A-B denotes C═C.

Preference is given to compounds of formula I wherein X is sulfur, oxygen, S═O, SO₂, NH, N(CH₃), C═O, or CH₂.

Moreover, preference is given to compounds of formula I wherein X is sulfur, oxygen, CH₂ or NH.

Particular preference is given to compounds of formula I wherein X is sulfur or CH₂.

Moreover, particular preference is given to compounds of formula I wherein X is sulfur.

Also, particular preference is given to compounds of formula I wherein X is CH₂.

Preference is given to compounds of formula I wherein R¹ is hydrogen, halogen, hydroxy, mercapto, cyano, nitro, C₁-C₆-alkyl, C₁-C₆-alkoxy, C₁-C₆-alkylthio, C₁-C₆-alkylsulfonyl, C₁-C₆-alkylsulfoxyl, C₁-C₆-alkoxycarbonyl, C(O)NR^{d}R^{e}, (SO₂)ₙNR^{d}R^{e}, wherein the carbon atoms in the aliphatic groups may be substituted by 1 to 3 groups R^{#}; or C(═NOR^{f})-Γₚ-R^{f}′, wherein R^{f}′ and R^{f} are each independently hydrogen or C₁-C₆-alkyl, Γ is oxygen, sulfur or NR^{f} and p is 0 or 1;

Particular preference is given to compounds of formula I wherein R¹ is hydrogen, hydroxy, mercapto, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxycarbonyl, aminosulfonyl (NH₂SO₂), C₁-C₆-alkylaminosulfonyl, or di(C₁-C₆-alkyl)aminosulfonyl.

Furthermore, particular preference is given to compounds of formula I wherein R¹ is hydrogen, hydroxy, mercapto, halogen, cyano, nitro, C₁-C₆-alkyl, C₁-C₆-haloalkyl, C₁-C₆-alkoxy, C₁-C₆-haloalkoxy, or C₁-C₆-haloalkylthio.

Also, particular preference is given to compounds of formula I wherein R¹ is hydrogen.

Also, particular preference is given to compounds of formula I wherein R¹ is halogen, preferably fluoro, chloro, or bromo, most preferably fluoro.

Also, particular preference is given to compounds of formula I wherein R¹ is cyano.

Also, particular preference is given to compounds of formula I wherein R¹ is hydroxy.

Also, particular preference is given to compounds of formula I wherein R¹ is mercapto.

Also, particular preference is given to compounds of formula I wherein R¹ is nitro.

Also, particular preference is given to compounds of formula I wherein R¹ is C₁-C₆-alkyl, preferably methyl.

Also, particular preference is given to compounds of formula I wherein R¹ is C₁-C₆-alkoxy, preferably methoxy.

Also, particular preference is given to compounds of formula I wherein R¹ is C₁-C₆-haloalkoxy, preferably trifluoromethoxy or difluoromethoxy, most preferably trifluoromethoxy.

Also, particular preference is given to compounds of formula I wherein R¹ is C₁-C₆-haloalkyl, preferably trifluoromethyl.

Also, particular preference is given to compounds of formula I wherein R¹ is C₁-C₆-haloalkylthio, preferably trifluoromethylthio or difluoromethylthio.

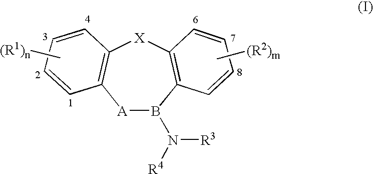

Furthermore, particular preference is given to compounds of formula I wherein R¹ is in the 1-, 2-, 4- and/or 3-position.

Moreover, particular preference is given to compounds of formula I wherein R¹ is in the 3-position.

Preference is given to compounds of formula I wherein R² is hydrogen, halogen, hydroxy, mercapto, amino, cyano, nitro, C₁-C₆-alkyl, C₁-C₆-alkoxy, C₁-C₆-alkylamino, di(C₁-C₆-alkyl)amino, C₁-C₆-alkylthio, C₂-C₆-alkenyl, C₂-C₆-alkenyloxy, C₂-C₆-alkenylamino, C₂-C₆-alkenylthio, C₂-C₆-alkynyl, C₂-C₆-alkynyloxy, C₂-C₆-alkynylamino, C₂-C₆-alkynylthio, C₁-C₆-alkylsulfonyl, C₁-C₆-alkylsulfoxyl, C₂-C₆-alkenylsulfonyl, C₂-C₆-alkynylsulfoxyl, formyl, C₁-C₆-alkylcarbonyl, hydroxycarbonyl, C₁-C₆-alkoxycarbonyl, carbonyloxy, C₁-C₆-alkylcarbonyloxy, C₃-C₆-cycloalkyl, C₃-C₆-cycloalkyl-C₁-C₄-alkyl, C₃-C₆-cycloalkylthio, C₃-C₆-cycloalkyl-C₁-C₄-alkylthio, wherein the carbon atoms in the aliphatic groups may be substituted by 1 to 3 groups R^{#}, or C(═NOR^{f})-Γ₁-R^{f}′, wherein R^{f}′ and R^{f} are each independently hydrogen or C₁-C₆-alkyl, Γ is oxygen, sulfur or NR^{f} and p is 0 or 1, or phenyl;

Particular preference is given to compounds of formula I wherein R² is hydrogen, hydroxy, mercapto, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₆-alkenyl, C₂-C₆-alkynyl, C₂-C₆-alkynylthio, C₁-C₆-alkylcarbonyl, C₁-C₆-alkoxycarbonyl, C₃-C₆-cycloalkyl, C₃-C₆-cycloalkyl-C₁-C₄-alkyl, C₃-C₆-cycloalkylthio, C₃-C₆-cycloalkyl-C₁-C₄-alkylthio, (CH₃)C═NOH, CH[═NO(C₁-C₆-alkyl)], CH═NOH, or (CH₃)C[═NO(C₁-C₆-alkyl)].

Moreover, particular preference is given to compounds of formula I wherein R² is hydrogen, hydroxy, mercapto, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₆-alkenyl, or C₂-C₆-alkynyl.

Also, particular preference is given to compounds of formula I wherein R² is hydrogen.

Also, particular preference is given to compounds of formula I wherein R² is hydroxy.

Also, particular preference is given to compounds of formula I wherein R² is mercapto.

Also, particular preference is given to compounds of formula I wherein R² is halogen, preferably fluoro, chloro or bromo, most preferably fluoro.

Also, particular preference is given to compounds of formula I wherein R² is cyano.

Also, particular preference is given to compounds of formula I wherein R² is C₁-C₆-alkyl, preferably methyl, ethyl, n-propyl, iso-propyl, n-butyl, iso-butyl, or tert.-butyl, most preferably methyl or ethyl, especially methyl.

Also, particular preference is given to compounds of formula I wherein R² is C₁-C₆-haloalkyl, preferably trifluoromethyl.

Also, particular preference is given to compounds of formula I wherein R² is C₁-C₆-alkoxy, preferably methoxy.

Also, particular preference is given to compounds of formula I wherein R² is C₁-C₆-haloalkoxy, preferably trifluoromethoxy or difluoromethoxy.

Also, particular preference is given to compounds of formula I wherein R² is C₁-C₆-alkylthio, preferably methylthio, ethylthio, n-propylthio, iso-propylthio, n-butylthio, iso-butylthio, or tert.-butylthio, most preferably methylthio or ethylthio, especially methylthio.

Also, particular preference is given to compounds of formula I wherein R² is C₁-C₆-haloalkylthio, preferably trifluoromethylthio or difluoromethylthio.

Also, particular preference is given to compounds of formula I wherein R² is C₂-C₆-alkenyl, preferably allyl.

Also, particular preference is given to compounds of formula I wherein R² is C₂-C₆-alkynyl, preferably propargyl.

Also, particular preference is given to compounds of formula I wherein R² is C₃-C₆-cycloalkyl, preferably cyclopropyl.

Also, particular preference is given to compounds of formula I wherein R² is C₃-C₆-cycloalkyl-C₁-C₄-alkyl, preferably cyclopropylmethyl.

Moreover, particular preference is given to compounds of formula I wherein R² is in the 6-, 7-, and/or 8-position.

Moreover, particular preference is given to compounds of formula I wherein R² is in the 8-position.

Preference is given to compounds of formula I wherein R^{b} is halogen, cyano, or nitro.

Preference is given to compounds of formula I wherein R³ and R⁴ together with the nitrogen atom to which they are attached form a saturated monocyclic 5- to 7-membered ringsystem containing 1 to 3 heteroatoms selected from nitrogen and oxygen, pyrrolidine, piperazine, homopiperazine, morpholine, or piperidine, wherein the carbon and/or nitrogen atoms are unsubstituted or substituted with any combination of hydrogen, amino, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-alkylamino, di(C₁-C₆-alkyl)amino, C₂-C₆-alkenylamino, C₂-C₆-alkynylamino, C₁-C₆-hydroxyalkyl, hydroxycarbonyl-C₁-C₄-alkyl, C₁-C₆-alkoxycarbonyl-C₁-C₄-alkyl, formyl-C₁-C₄-alkyl, formyl-C₁-C₄-alkoxy, C₁-C₆-alkylcarbonyl-C₁-C₄-alkoxy, C₃-C₆-cycloalkyl, which is bonded directly or through an oxygen, sulfur or C₁-C₆-alkyl linkage, or C₅-C₈-cycloalkenyl, wherein the carbon atoms in these aliphatic groups can be substituted by 1 to 4 groups selected from halogen, cyano, hydroxy and nitro.

Particular preference is given to compounds of formula I wherein R³ and R⁴ together with the nitrogen atom to which they are attached form a piperazine or homopiperazine ring of formula IX wherein R^{z} is hydrogen, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-hydroxyalkyl, hydroxycarbonyl-C₁-C₄-alkyl, C₁-C₆-alkoxycarbonyl-C₁-C₄-alkyl, formyl-C₁-C₄-alkyl, formyl-C₁-C₄-alkoxy, C₁-C₆-alkylcarbonyl-C₁-C₄-alkoxy, C₃-C₆-cycloalkyl, which is bonded directly or through an oxygen, sulfur or C₁-C₆-alkyl, C₅-C₈-cycloalkenyl linkage, wherein the carbon atoms in these aliphatic groups can be substituted by 1 to 4 groups selected from halogen, cyano, hydroxy and nitro, and o is 1 or 2.

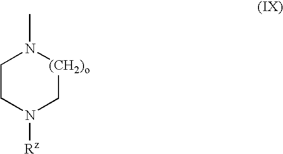

Particular preference further is given to compounds of formula I wherein R³ and R⁴ together with the nitrogen atom to which they are attached form a ring of formula IX wherein R^{z} is C₁-C₆-alkyl, C₁-C₆-haloalkyl, C₁-C₆-alkoxy, C₁-C₆-alkoxy-C₁-C₄-alkyl, C₂-C₆-alkenyl, C₂-C₆-alkynyl, C₃-C₆-cycloalkyl, C₃-C₆-cycloalkyl-C₁-C₄-alkyl, C₃-C₆-cycloalkoxy, C₃-C₆-cycloalkyl-C₁-C₄-alkoxy, phenyl, or benzyl.

Most preferred are compounds of formula I wherein R^{z} is C₁-C₆-alkyl, C₁-C₆-haloalkyl, C₂-C₆-alkenyl, C₂-C₆-alkynyl, C₃-C₆-cycloalkyl, or C₃-C₆-cycloalkyl-C₁-C₄-alkyl.

Also, particular preference is given to compounds of formula I wherein R^{z} is C₁-C₆-alkyl, preferably methyl, ethyl, n-propyl, iso-propyl, n-butyl, iso-butyl, tert.-butyl, n-hexyl, or cyclohexyl.

Also, particular preference is given to compounds of formula I wherein R^{z} is methyl.

Also, particular preference is given to compounds of formula I wherein R^{z} is ethyl.

Also, particular preference is given to compounds of formula I wherein R^{z} is C₁-C₆-haloalkyl, preferably 1,1,1-trifluoro-eth-2-yl, 1,1-difluoro-1-chloro-eth-2-yl, 1-fluoro-1,1-dichloro-eth-2-yl, 1,1,1-trichloro-eth-2-yl, most preferably 1,1,1-trifluoro-eth-2-yl.

Also, particular preference is given to compounds of formula I wherein R^{z} is C₂-C₆-alkenyl, preferably allyl.

Also, particular preference is given to compounds of formula I wherein R^{z} is C₂-C₆-alkynyl, preferably propargyl.

Also, particular preference is given to compounds of formula I wherein R^{z} is C₃-C₆-cycloalkyl, preferably cyclopropyl.

Also, particular preference is given to compounds of formula I wherein R^{z} is C₃-C₆-cycloalkyl-C₁-C₄-alkyl, preferably cyclopropylmethyl.

Moreover, particular preference is given to compounds of formula I wherein R³ and R⁴ together with the nitrogen atom to which they are attached form a ring of formula IX wherein o is 1.

Furthermore, preference is given to compounds of formula I wherein R^{a}, R^{d}, and R^{e} are each independently hydrogen, C₁-C₆-alkyl, C₁-C₆-alkenyl, C₁-C₆-alkynyl or
phenyl or benzyl, each unsubstituted or substituted with any combination of 1 to 5 halogen, or 1 to 3 C₁-C₆-alkyl, C₁-C₆-haloalkyl, C₁-C₆-alkylthio, or C₁-C₆-alkoxy.

Moreover, preference is given to compounds of formula I wherein R^{b} and R^{c} are each independently hydrogen, hydroxy, amino, C₁-C₆-alkyl, C₁-C₆-hydroxyalkyl, C₁-C₆-alkylamino, di(C₁-C₆-alkyl)amino, or phenyl, unsubstituted or substituted with any combination of 1 to 5 halogen, or 1 to 3 C₁-C₆-alkyl, C₁-C₆-haloalkyl, C₁-C₆-alkoxy or C₁-C₆-haloalkoxy groups,
or CR^{b}R^{c} represents C═O or C═CR^{j}R^{k}, wherein R^{j} and R^{k} each independently are hydrogen, halogen, cyano, C₁-C₆-alkyl, C₁-C₆-haloalkyl, or C₃-C₆-cycloalkyl.

Preference is given to compounds of formula I wherein m is 0, 1, or 2.

Particular preference is given to compounds of formula I wherein m is 1.

Preference is given to compounds of formula I wherein n is 0, 1, or 2.

Particular preference is given to compounds of formula I wherein n is 1.

With respect to their use, particular preference is given to the compounds I-1 compiled in the tables below. Moreover, the groups mentioned for a substituent in the tables are on their own, independently of the combination in which they are mentioned, a particularly preferred embodiment of the substituent in question.

With respect to their use, particular preference is also given to the maleic acid, dimaleic acid, fumaric acid, difumaric acid, methane sulfenic acid, methane sulfonic acid, and succinic acid adducts of the compounds of the tables below.

Some of the compounds of formula I are new. These are also subject-matter of this invention.

Table 1

Compounds of the formula I-1 wherein A-B denotes C—C, X is sulfur, n is zero, and the combination of (R²)ₘ, R^{z} and o corresponds in each case to a row of Table A. 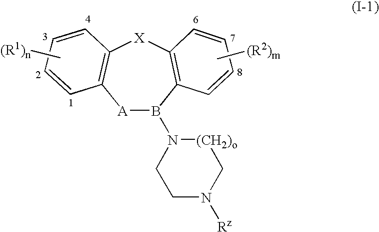
Table 2

Compounds of the formula I-1 wherein A-B denotes C—C, X is sulfur, n is 1, R¹ is 1-fluoro and the combination of (R²)ₘ, R^{z} and o corresponds in each case to a row of Table A.

Table 3

Compounds of the formula I-1 wherein A-B denotes C—C, X is sulfur, n is 1, R¹ is 1-chloro and the combination of (R²)ₘ, R^{z} and o corresponds in each case to a row of Table A.

Table 4

Compounds of the formula I-1 wherein A-B denotes C—C, X is sulfur, n is 1, R¹ is 1-bromo and the combination of (R²)ₘ, R^{z} and o corresponds in each case to a row of Table A.

Table 5

Compounds of the formula I-1 wherein A-B denotes C—C, X is sulfur, n is 1, R¹ is 1-hydroxy and the combination of (R²)ₘ, R^{z} and o corresponds in each case to a row of Table A.

Table 6

Compounds of the formula I-1 wherein A-B denotes C—C, X is sulfur, n is 1, R¹ is 1-mercapto and the combination of (R²)ₘ, R^{z} and o corresponds in each case to a row of Table A.

Table 7

Compounds of the formula I-1 wherein A-B denotes C—C, X is sulfur, n is 1, R¹ is 1-cyano and the combination of (R²)ₘ, R^{z} and o corresponds in each case to a row of Table A.

Table 8

Compounds of the formula I-1 wherein A-B denotes C—C, X is sulfur, n is 1, R¹ is 1-nitro and the combination of (R²)ₘ, R^{z} and o corresponds in each case to a row of Table A.

Table 9

Compounds of the formula I-1 wherein A-B denotes C—C, X is sulfur, n is 1, R¹ is 1-methyl and the combination of (R²)ₘ, R^{z} and o corresponds in each case to a row of Table A.

Table 10

Compounds of the formula I-1 wherein A-B denotes C—C, X is sulfur, n is 1, R¹ is 1-trifluoromethyl and the combination of (R²)ₘ, R^{z} and o corresponds in each case to a row of Table A.

Table 11

Compounds of the formula I-1 wherein A-B denotes C—C, X is sulfur, n is 1, R¹ is 1-methoxy and the combination of (R²)ₘ, R^{z} and o corresponds in each case to a row of Table A.

Table 12

Compounds of the formula I-1 wherein A-B denotes C—C, X is sulfur, n is 1, R¹ is 1-trifluoromethoxy and the combination of (R²)ₘ, R^{z} and o corresponds in each case to a row of Table A.

Table 13

Compounds of the formula I-1 wherein A-B denotes C—C, X is sulfur, n is 1, R¹ is 1-trifluoromethylthio and the combination of (R²)ₘ, R^{z} and o corresponds in each case to a row of Table A.

Table 14

Compounds of the formula I-1 wherein A-B denotes C—C, X is sulfur, n is 1, R¹ is 2-fluoro and the combination of (R²)ₘ, R^{z} and o corresponds in each case to a row of Table A.

Table 15

Compounds of the formula I-1 wherein A-B denotes C—C, X is sulfur, n is 1, R¹ is 2-chloro and the combination of (R²)ₘ, R^{z} and o corresponds in each case to a row of Table A.

Table 16

Compounds of the formula I-1 wherein A-B denotes C—C, X is sulfur, n is 1, R¹ is 2-bromo and the combination of (R²)ₘ, R^{z} and o corresponds in each case to a row of Table A.

Table 17

Compounds of the formula I-1 wherein A-B denotes C—C, X is sulfur, n is 1, R¹ is 2-hydroxy and the combination of (R²)ₘ, R^{z} and o corresponds in each case to a row of Table A.

Table 18

Compounds of the formula I-1 wherein A-B denotes C—C, X is sulfur, n is 1, R¹ is 2-mercapto and the combination of (R²)ₘ, R^{z} and o corresponds in each case to a row of Table A.

Table 19

Compounds of the formula I-1 wherein A-B denotes C—C, X is sulfur, n is 1, R¹ is 2-cyano and the combination of (R²)ₘ, R^{z} and o corresponds in each case to a row of Table A.

Table 20

Compounds of the formula I-1 wherein A-B denotes C—C, X is sulfur, n is 1, R¹ is 2-nitro and the combination of (R²)ₘ, R^{z} and o corresponds in each case to a row of Table A.

Table 21

Compounds of the formula I-1 wherein A-B denotes C—C, X is sulfur, n is 1, R¹ is 2-methyl and the combination of (R²)ₘ, R^{z} and o corresponds in each case to a row of Table A.

Table 22

Compounds of the formula I-1 wherein A-B denotes C—C, X is sulfur, n is 1, R¹ is 2-trifluoromethyl and the combination of (R²)ₘ, R^{z} and o corresponds in each case to a row of Table A.

Table 23

Compounds of the formula I-1 wherein A-B denotes C—C, X is sulfur, n is 1, R¹ is 2-methoxy and the combination of (R²)ₘ, R^{z} and o corresponds in each case to a row of Table A.

Table 24

Compounds of the formula I-1 wherein A-B denotes C—C, X is sulfur, n is 1, R¹ is 2-trifluoromethoxy and the combination of (R²)ₘ, R^{z} and o corresponds in each case to a row of Table A.

Table 25

Compounds of the formula I-1 wherein A-B denotes C—C, X is sulfur, n is 1, R¹ is 2-trifluoromethylthio and the combination of (R²)ₘ, R^{z} and o corresponds in each case to a row of Table A.

Table 26

Compounds of the formula I-1 wherein A-B denotes C—C, X is sulfur, n is 1, R¹ is 3-fluoro and the combination of (R²)ₘ, R^{z} and o corresponds in each case to a row of Table A.

Table 27

Compounds of the formula I-1 wherein A-B denotes C—C, X is sulfur, n is 1, R¹ is 3-chloro and the combination of (R²)ₘ, R^{z} and o corresponds in each case to a row of Table A.

Table 28

Compounds of the formula I-1 wherein A-B denotes C—C, X is sulfur, n is 1, R¹ is 3-bromo and the combination of (R²)ₘ, R^{z} and o corresponds in each case to a row of Table A.

Table 29

Compounds of the formula I-1 wherein A-B denotes C—C, X is sulfur, n is 1, R¹ is 3-hydroxy and the combination of (R²)ₘ, R^{z} and o corresponds in each case to a row of Table A.

Table 30

Compounds of the formula I-1 wherein A-B denotes C—C, X is sulfur, n is 1, R¹ is 3-mercapto and the combination of (R²)ₘ, R^{z} and o corresponds in each case to a row of Table A.

Table 31

Compounds of the formula I-1 wherein A-B denotes C—C, X is sulfur, n is 1, R¹ is 3-cyano and the combination of (R²)ₘ, R^{z} and o corresponds in each case to a row of Table A.

Table 32

Compounds of the formula I-1 wherein A-B denotes C—C, X is sulfur, n is 1, R¹ is 3-nitro and the combination of (R²)ₘ, R^{z} and o corresponds in each case to a row of Table A.

Table 33

Compounds of the formula I-1 wherein A-B denotes C—C, X is sulfur, n is 1, R¹ is 3-methyl and the combination of (R²)ₘ, R^{z} and o corresponds in each case to a row of Table A.

Table 34

Compounds of the formula I-1 wherein A-B denotes C—C, X is sulfur, n is 1, R¹ is 3-trifluoromethyl and the combination of (R²)ₘ, R^{z} and o corresponds in each case to a row of Table A.

Table 35

Compounds of the formula I-1 wherein A-B denotes C—C, X is sulfur, n is 1, R¹ is 3-methoxy and the combination of (R²)ₘ, R^{z} and o corresponds in each case to a row of Table A.

Table 36

Compounds of the formula I-1 wherein A-B denotes C—C, X is sulfur, n is 1, R¹ is 3-trifluoromethoxy and the combination of (R²)ₘ, R^{z} and o corresponds in each case to a row of Table A.

Table 37

Compounds of the formula I-1 wherein A-B denotes C—C, X is sulfur, n is 1, R¹ is 3-trifluoromethylthio and the combination of (R²)ₘ, R^{z} and o corresponds in each case to a row of Table A.

Table 38

Compounds of the formula I-1 wherein A-B denotes C—C, X is sulfur, n is 1, R¹ is 4-fluoro and the combination of (R²)ₘ, R^{z} and o corresponds in each case to a row of Table A.

Table 39

Compounds of the formula I-1 wherein A-B denotes C—C, X is sulfur, n is 1, R¹ is 4-chloro and the combination of (R²)ₘ, R^{z} and o corresponds in each case to a row of Table A.

Table 40

Compounds of the formula I-1 wherein A-B denotes C—C, X is sulfur, n is 1, R¹ is 4-bromo and the combination of (R²)ₘ, R^{z} and o corresponds in each case to a row of Table A.

Table 41

Compounds of the formula I-1 wherein A-B denotes C—C, X is sulfur, n is 1, R¹ is 4-hydroxy and the combination of (R²)ₘ, R^{z} and o corresponds in each case to a row of Table A.

Table 42

Compounds of the formula I-1 wherein A-B denotes C—C, X is sulfur, n is 1, R¹ is 4-mercapto and the combination of (R²)ₘ, R^{z} and o corresponds in each case to a row of Table A.

Table 43

Compounds of the formula I-1 wherein A-B denotes C—C, X is sulfur, n is 1, R¹ is 4-cyano and the combination of (R²)ₘ, R^{z} and o corresponds in each case to a row of Table A.

Table 44

Compounds of the formula I-1 wherein A-B denotes C—C, X is sulfur, n is 1, R¹ is 4-nitro and the combination of (R²)ₘ, R^{z} and o corresponds in each case to a row of Table A.

Table 45

Compounds of the formula I-1 wherein A-B denotes C—C, X is sulfur, n is 1, R¹ is 4-methyl and the combination of (R²)ₘ, R^{z} and o corresponds in each case to a row of Table A.

Table 46

Compounds of the formula I-1 wherein A-B denotes C—C, X is sulfur, n is 1, R¹ is 4-trifluoromethyl and the combination of (R²)ₘ, R^{z} and o corresponds in each case to a row of Table A.

Table 47

Compounds of the formula I-1 wherein A-B denotes C—C, X is sulfur, n is 1, R¹ is 4-methoxy and the combination of (R²)ₘ, R^{z} and o corresponds in each case to a row of Table A.

Table 48

Compounds of the formula I-1 wherein A-B denotes C—C, X is sulfur, n is 1, R¹ is 4-trifluoromethoxy and the combination of (R²)ₘ, R^{z} and o corresponds in each case to a row of Table A.

Table 49

Compounds of the formula I-1 wherein A-B denotes C—C, X is sulfur, n is 1, R¹ is 4-trifluoromethylthio and the combination of (R²)ₘ, R^{z} and o corresponds in each case to a row of Table A.

Table 50

Compounds of the formula I-1 wherein A-B denotes C—C, X is sulfur, (R¹)ₙ is 2,3-difluoro, and the combination of (R²)ₘ, R^{z} and o corresponds in each case to a row of Table A.

Table 51

Compounds of the formula I-1 wherein A-B denotes C—C, X is sulfur, (R¹)ₙ is 2-fluoro-3-chloro, and the combination of (R²)ₘ, R^{z} and o corresponds in each case to a row of Table A.

Table 52

Compounds of the formula I-1 wherein A-B denotes C—C, X is sulfur, (R¹)ₙ is 2-chloro-3-fluoro, and the combination of (R²)ₘ, R^{z} and o corresponds in each case to a row of Table A.

Table 53

Compounds of the formula I-1 wherein A-B denotes C—C, X is sulfur, (R¹)ₙ is 2,3-dimethoxy, and the combination of (R²)ₘ, R^{z} and o corresponds in each case to a row of Table A.

Table 54

Compounds of the formula I-1 wherein A-B denotes C—C, X is methylene, n is zero, and the combination of (R²)ₘ, R^{z} and o corresponds in each case to a row of Table A.

Table 55

Compounds of the formula I-1 wherein A-B denotes C—C, X is methylene, n is 1, R¹ is 1-fluoro and the combination of (R²)ₘ, R^{z} and o corresponds in each case to a row of Table A.

Table 56

Compounds of the formula I-1 wherein A-B denotes C—C, X is methylene, n is 1, R¹ is 1-chloro and the combination of (R²)ₘ, R^{z} and o corresponds in each case to a row of Table A.

Table 57

Compounds of the formula I-1 wherein A-B denotes C—C, X is methylene, n is 1, R¹ is 1-bromo and the combination of (R²)ₘ, R^{z} and o corresponds in each case to a row of Table A.

Table 58

Compounds of the formula I-1 wherein A-B denotes C—C, X is methylene, n is 1, R¹ is 1-hydroxy and the combination of (R²)ₘ, R^{z} and o corresponds in each case to a row of Table A.

Table 59

Compounds of the formula I-1 wherein A-B denotes C—C, X is methylene, n is 1, R¹ is 1-mercapto and the combination of (R²)ₘ, R^{z} and o corresponds in each case to a row of Table A.

Table 60

Compounds of the formula I-1 wherein A-B denotes C—C, X is methylene, n is 1, R¹ is 1-cyano and the combination of (R²)ₘ, R^{z} and o corresponds in each case to a row of Table A.

Table 61

Compounds of the formula I-1 wherein A-B denotes C—C, X is methylene, n is 1, R¹ is 1-nitro and the combination of (R²)ₘ, R^{z} and o corresponds in each case to a row of Table A.

Table 62

Compounds of the formula I-1 wherein A-B denotes C—C, X is methylene, n is 1, R¹ is 1-methyl and the combination of (R²)ₘ, R^{z} and o corresponds in each case to a row of Table A.

Table 63

Compounds of the formula I-1 wherein A-B denotes C—C, X is methylene, n is 1, R¹ is 1-trifluoromethyl and the combination of (R²)ₘ, R^{z} and o corresponds in each case to a row of Table A.

Table 64

Compounds of the formula I-1 wherein A-B denotes C—C, X is methylene, n is 1, R¹ is 1-methoxy and the combination of (R²)ₘ, R^{z} and o corresponds in each case to a row of Table A.

Table 65

Compounds of the formula I-1 wherein A-B denotes C—C, X is methylene, n is 1, R¹ is 1-trifluoromethoxy and the combination of (R²)ₘ, R^{z} and o corresponds in each case to a row of Table A.

Table 66

Compounds of the formula I-1 wherein A-B denotes C—C, X is methylene, n is 1, R¹ is 1-trifluoromethylthio and the combination of (R²)ₘ, R^{z} and o corresponds in each case to a row of Table A.

Table 67

Compounds of the formula I-1 wherein A-B denotes C—C, X is methylene, n is 1, R¹ is 2-fluoro and the combination of (R²)ₘ, R^{z} and o corresponds in each case to a row of Table A.

Table 68

Compounds of the formula I-1 wherein A-B denotes C—C, X is methylene, n is 1, R¹ is 2-chloro and the combination of (R²)ₘ, R^{z} and o corresponds in each case to a row of Table A.

Table 69

Compounds of the formula I-1 wherein A-B denotes C—C, X is methylene, n is 1, R¹ is 2-bromo and the combination of (R²)ₘ, R^{z} and o corresponds in each case to a row of Table A.

Table 70

Compounds of the formula I-1 wherein A-B denotes C—C, X is methylene, n is 1, R¹ is 2-hydroxy and the combination of (R²)ₘ, R^{z} and o corresponds in each case to a row of Table A.

Table 71

Compounds of the formula I-1 wherein A-B denotes C—C, X is methylene, n is 1, R¹ is 2-mercapto and the combination of (R²)ₘ, R^{z} and o corresponds in each case to a row of Table A.

Table 72

Compounds of the formula I-1 wherein A-B denotes C—C, X is methylene, n is 1, R¹ is 2-cyano and the combination of (R²)ₘ, R^{z} and o corresponds in each case to a row of Table A.

Table 73

Compounds of the formula I-1 wherein A-B denotes C—C, X is methylene, n is 1, R¹ is 2-nitro and the combination of (R²)ₘ, R^{z} and o corresponds in each case to a row of Table A.

Table 74

Compounds of the formula I-1 wherein A-B denotes C—C, X is methylene, n is 1, R¹ is 2-methyl and the combination of (R²)ₘ, R^{z} and o corresponds in each case to a row of Table A.

Table 75

Compounds of the formula I-1 wherein A-B denotes C—C, X is methylene, n is 1, R¹ is 2-trifluoromethyl and the combination of (R²)ₘ, R^{z} and o corresponds in each case to a row of Table A.

Table 76

Compounds of the formula I-1 wherein A-B denotes C—C, X is methylene, n is 1, R¹ is 2-methoxy and the combination of (R²), R^{z} and o corresponds in each case to a row of Table A.

Table 77

Compounds of the formula I-1 wherein A-B denotes C—C, X is methylene, n is 1, R¹ is 2-trifluoromethoxy and the combination of (R²)ₘ, R^{z} and o corresponds in each case to a row of Table A.

Table 78

Compounds of the formula I-1 wherein A-B denotes C—C, X is methylene, n is 1, R¹ is 2-trifluoromethylthio and the combination of (R²)ₘ, R^{z} and o corresponds in each case to a row of Table A.

Table 79

Compounds of the formula I-1 wherein A-B denotes C—C, X is methylene, n is 1, R¹ is 3-fluoro and the combination of (R²)ₘ, R^{z} and o corresponds in each case to a row of Table A.

Table 80

Compounds of the formula I-1 wherein A-B denotes C—C, X is methylene, n is 1, R¹ is 3-chloro and the combination of (R²)ₘ, R^{z} and o corresponds in each case to a row of Table A.

Table 81

Compounds of the formula I-1 wherein A-B denotes C—C, X is methylene, n is 1, R¹ is 3-bromo and the combination of (R²)ₘ, R^{z} and o corresponds in each case to a row of Table A.

Table 82

Compounds of the formula I-1 wherein A-B denotes C—C, X is methylene, n is 1, R¹ is 3-hydroxy and the combination of (R²)ₘ, R^{z} and o corresponds in each case to a row of Table A.

Table 83

Compounds of the formula I-1 wherein A-B denotes C—C, X is methylene, n is 1, R¹ is 3-mercapto and the combination of (R²)ₘ, R^{z} and o corresponds in each case to a row of Table A.

Table 84

Compounds of the formula I-1 wherein A-B denotes C—C, X is methylene, n is 1, R¹ is 3-cyano and the combination of (R²)ₘ, R^{z} and o corresponds in each case to a row of Table A.

Table 85

Compounds of the formula I-1 wherein A-B denotes C—C, X is methylene, n is 1, R¹ is 3-nitro and the combination of (R²)ₘ, R^{z} and o corresponds in each case to a row of Table A.

Table 86

Compounds of the formula I-1 wherein A-B denotes C—C, X is methylene, n is 1, R¹ is 3-methyl and the combination of (R²)ₘ, R^{z} and o corresponds in each case to a row of Table A.

Table 87

Compounds of the formula I-1 wherein A-B denotes C—C, X is methylene, n is 1, R¹ is 3-trifluoromethyl and the combination of (R²)ₘ, R^{z} and o corresponds in each case to a row of Table A.

Table 88

Compounds of the formula I-1 wherein A-B denotes C—C, X is methylene, n is 1, R¹ is 3-methoxy and the combination of (R²)ₘ, R^{z} and o corresponds in each case to a row of Table A.

Table 89

Compounds of the formula I-1 wherein A-B denotes C—C, X is methylene, n is 1, R¹ is 3-trifluoromethoxy and the combination of (R²)ₘ, R^{z} and o corresponds in each case to a row of Table A.

Table 90

Compounds of the formula I-1 wherein A-B denotes C—C, X is methylene, n is 1, R¹ is 3-trifluoromethylthio and the combination of (R²)ₘ, R^{z} and o corresponds in each case to a row of Table A.

Table 91

Compounds of the formula I-1 wherein A-B denotes C—C, X is methylene, n is 1, R¹ is 4-fluoro and the combination of (R²)ₘ, R^{z} and o corresponds in each case to a row of Table A.

Table 92

Compounds of the formula I-1 wherein A-B denotes C—C, X is methylene, n is 1, R¹ is 4-chloro and the combination of (R²)ₘ, R^{z} and o corresponds in each case to a row of Table A.

Table 93

Compounds of the formula I-1 wherein A-B denotes C—C, X is methylene, n is 1, R¹ is 4-bromo and the combination of (R²)ₘ, R^{z} and o corresponds in each case to a row of Table A.

Table 94

Compounds of the formula I-1 wherein A-B denotes C—C, X is methylene, n is 1, R¹ is 4-hydroxy and the combination of (R²)ₘ, R^{z} and o corresponds in each case to a row of Table A.

Table 95

Compounds of the formula I-1 wherein A-B denotes C—C, X is methylene, n is 1, R¹ is 4-mercapto and the combination of (R²)ₘ, R^{z} and o corresponds in each case to a row of Table A.

Table 96

Compounds of the formula I-1 wherein A-B denotes C—C, X is methylene, n is 1, R¹ is 4-cyano and the combination of (R²)ₘ, —R^{z} and o corresponds in each case to a row of Table A.

Table 97

Compounds of the formula I-1 wherein A-B denotes C—C, X is methylene, n is 1, R¹ is 4-nitro and the combination of (R²)ₘ, R^{z} and o corresponds in each case to a row of Table A.

Table 98

Compounds of the formula I-1 wherein A-B denotes C—C, X is methylene, n is 1, R¹ is 4-methyl and the combination of (R²)ₘ, R^{z} and o corresponds in each case to a row of Table A.

Table 99

Compounds of the formula I-1 wherein A-B denotes C—C, X is methylene, n is 1, R¹ is 4-trifluoromethyl and the combination of (R²)ₘ, R^{z} and o corresponds in each case to a row of Table A.

Table 100

Compounds of the formula I-1 wherein A-B denotes C—C, X is methylene, n is 1, R¹ is 4-methoxy and the combination of (R²)ₘ, R^{z} and o corresponds in each case to a row of Table A.

Table 101

Compounds of the formula I-1 wherein A-B denotes C—C, X is methylene, n is 1, R¹ is 4-trifluoromethoxy and the combination of (R²)ₘ, R^{z} and o corresponds in each case to a row of Table A.

Table 102

Compounds of the formula I-1 wherein A-B denotes C—C, X is methylene, n is 1, R¹ is 4-trifluoromethylthio and the combination of (R²)ₘ, R^{z} and o corresponds in each case to a row of Table A.

Table 103

Compounds of the formula I-i wherein A-B denotes C—C, X is methylene, (R¹)ₙ is 2,3-difluoro, and the combination of (R²)ₘ, R^{z} and o corresponds in each case to a row of Table A.

Table 104

Compounds of the formula I-1 wherein A-B denotes C—C, X is methylene, (R¹)ₙ is 2-fluoro-3-chloro, and the combination of (R²)ₘ, R^{z} and o corresponds in each case to a row of Table A.

Table 105

Compounds of the formula I-1 wherein A-B denotes C—C, X is methylene, (R¹)ₙ is 2-chloro-3-fluoro, and the combination of (R²)ₘ, R^{z} and o corresponds in each case to a row of Table A.

Table 106

Compounds of the formula I-1 wherein A-B denotes C—C, X is methylene, (R¹)ₙ is 2,3-dimethoxy, and the combination of (R²)ₘ, R^{z} and o corresponds in each case to a row of Table A.

Table 107

Compounds of the formula I-1 wherein A-B denotes C—C, X is oxygen, n is zero, and the combination of (R²)ₘ, R^{z} and o corresponds in each case to a row of Table A.

Table 108

Compounds of the formula I-1 wherein A-B denotes C—C, X is oxygen, n is 1, R¹ is 1-fluoro and the combination of (R²)ₘ, R^{z} and o corresponds in each case to a row of Table A.

Table 109

Compounds of the formula I-1 wherein A-B denotes C—C, X is oxygen, n is 1, R¹ is 1-chloro and the combination of (R²)ₘ, R^{z} and o corresponds in each case to a row of Table A.

Table 110

Compounds of the formula I-1 wherein A-B denotes C—C, X is oxygen, n is 1, R¹ is 1-bromo and the combination of (R²)ₘ, R^{z} and o corresponds in each case to a row of Table A.

Table 111

Compounds of the formula I-1 wherein A-B denotes C—C, X is oxygen, n is 1, R¹ is 1-hydroxy and the combination of (R²)ₘ, R^{z} and o corresponds in each case to a row of Table A.

Table 112

Compounds of the formula I-1 wherein A-B denotes C—C, X is oxygen, n is 1, R¹ is 1-mercapto and the combination of (R²)ₘ, R^{z} and o corresponds in each case to a row of Table A.

Table 113

Compounds of the formula I-1 wherein A-B denotes C—C, X is oxygen, n is 1, R¹ is 1-cyano and the combination of (R²)ₘ, R^{z} and o corresponds in each case to a row of Table A.

Table 114

Compounds of the formula I-1 wherein A-B denotes C—C, X is oxygen, n is 1, R¹ is 1-nitro and the combination of (R²)ₘ, R^{z} and o corresponds in each case to a row of Table A.

Table 115

Compounds of the formula I-1 wherein A-B denotes C—C, X is oxygen, n is 1, R¹ is 1-methyl and the combination of (R²)ₘ, R^{z} and o corresponds in each case to a row of Table A.

Table 116

Compounds of the formula I-1 wherein A-B denotes C—C, X is oxygen, n is 1, R¹ is 1-trifluoromethyl and the combination of (R²)ₘ, R^{z} and o corresponds in each case to a row of Table A.

Table 117

Compounds of the formula I-1 wherein A-B denotes C—C, X is oxygen, n is 1, R¹ is 1-methoxy and the combination of (R²)ₘ, R^{z} and o corresponds in each case to a row of Table A.

Table 118

Compounds of the formula I-1 wherein A-B denotes C—C, X is oxygen, n is 1, R¹ is 1-trifluoromethoxy and the combination of (R²)ₘ, R^{z} and o corresponds in each case to a row of Table A.

Table 119

Compounds of the formula I-1 wherein A-B denotes C—C, X is oxygen, n is 1, R¹ is 1-trifluoromethylthio and the combination of (R²)ₘ, R^{z} and o corresponds in each case to a row of Table A.

Table 120

Compounds of the formula I-1 wherein A-B denotes C—C, X is oxygen, n is 1, R¹ is 2-fluoro and the combination of (R²)ₘ, R^{z} and o corresponds in each case to a row of Table A.

Table 121

Compounds of the formula I-1 wherein A-B denotes C—C, X is oxygen, n is 1, R¹ is 2-chloro and the combination of (R²)ₘ, R^{z} and o corresponds in each case to a row of Table A.

Table 122

Compounds of the formula I-1 wherein A-B denotes C—C, X is oxygen, n is 1, R¹ is 2-bromo and the combination of (R²)ₘ, R^{z} and o corresponds in each case to a row of Table A.

Table 123

Compounds of the formula I-1 wherein A-B denotes C—C, X is oxygen, n is 1, R¹ is 2-hydroxy and the combination of (R²)ₘ, R^{z} and o corresponds in each case to a row of Table A.

Table 124

Compounds of the formula I-1 wherein A-B denotes C—C, X is oxygen, n is 1, R¹ is 2-mercapto and the combination of (R²)ₘ, R^{z} and o corresponds in each case to a row of Table A.

Table 125

Compounds of the formula I-1 wherein A-B denotes C—C, X is oxygen, n is 1, R¹ is 2-cyano and the combination of (R²)ₘ, R^{z} and o corresponds in each case to a row of Table A.

Table 126

Compounds of the formula I-1 wherein A-B denotes C—C, X is oxygen, n is 1, R¹ is 2-nitro and the combination of (R²)ₘ, R^{z} and o corresponds in each case to a row of Table A.

Table 127

Compounds of the formula I-1 wherein A-B denotes C—C, X is oxygen, n is 1, R¹ is 2-methyl and the combination of (R²)ₘ, R^{z} and o corresponds in each case to a row of Table A.

Table 128

Compounds of the formula I-1 wherein A-B denotes C—C, X is oxygen, n is 1, R¹ is 2-trifluoromethyl and the combination of (R²)ₘ, R^{z} and o corresponds in each case to a row of Table A.

Table 129

Compounds of the formula I-1 wherein A-B denotes C—C, X is oxygen, n is 1, R¹ is 2-methoxy and the combination of (R²)ₘ, R^{z} and o corresponds in each case to a row of Table A.

Table 130

Compounds of the formula I-1 wherein A-B denotes C—C, X is oxygen, n is 1, R¹ is 2-trifluoromethoxy and the combination of (R²)ₘ, R^{z} and o corresponds in each case to a row of Table A.

Table 131

Compounds of the formula I-1 wherein A-B denotes C—C, X is oxygen, n is 1, R¹ is 2-trifluoromethylthio and the combination of (R²)ₘ, R^{z} and o corresponds in each case to a row of Table A.

Table 132

Compounds of the formula I-1 wherein A-B denotes C—C, X is oxygen, n is 1, R¹ is 3-fluoro and the combination of (R²)ₘ, R^{z} and o corresponds in each case to a row of Table A.

Table 133

Compounds of the formula I-1 wherein A-B denotes C—C, X is oxygen, n is 1, R¹ is 3-chloro and the combination of (R²)ₘ, R^{z} and o corresponds in each case to a row of Table A.

Table 134

Compounds of the formula I-1 wherein A-B denotes C—C, X is oxygen, n is 1, R¹ is 3-bromo and the combination of (R²)ₘ, R^{z} and o corresponds in each case to a row of Table A.

Table 135

Compounds of the formula I-1 wherein A-B denotes C—C, X is oxygen, n is 1, R¹ is 3-hydroxy and the combination of (R²)ₘ, R^{z} and o corresponds in each case to a row of Table A.

Table 136

Compounds of the formula I-1 wherein A-B denotes C—C, X is oxygen, n is 1, R¹ is 3-mercapto and the combination of (R²)ₘ, R^{z} and o corresponds in each case to a row of Table A.

Table 137

Compounds of the formula I-1 wherein A-B denotes C—C, X is oxygen, n is 1, R¹ is 3-cyano and the combination of (R²)ₘ, R^{z} and o corresponds in each case to a row of Table A.

Table 138

Compounds of the formula I-1 wherein A-B denotes C—C, X is oxygen, n is 1, R¹ is 3-nitro and the combination of (R²)ₘ, R^{z} and o corresponds in each case to a row of Table A.

Table 139

Compounds of the formula I-1 wherein A-B denotes C—C, X is oxygen, n is 1, R¹ is 3-methyl and the combination of (R²)ₘ, R^{z} and o corresponds in each case to a row of Table A.

Table 140

Compounds of the formula I-1 wherein A-B denotes C—C, X is oxygen, n is 1, R¹ is 3-trifluoromethyl and the combination of (R²), R^{z} and o corresponds in each case to a row of Table A.

Table 141

Compounds of the formula I-1 wherein A-B denotes C—C, X is oxygen, n is 1, R¹ is 3-methoxy and the combination of (R²)ₘ, R^{z} and o corresponds in each case to a row of Table A.

Table 142

Compounds of the formula I-1 wherein A-B denotes C—C, X is oxygen, n is 1, R¹ is 3-trifluoromethoxy and the combination of (R²)ₘ, R^{z} and o corresponds in each case to a row of Table A.

Table 143

Compounds of the formula I-1 wherein A-B denotes C—C, X is oxygen, n is 1, R¹ is 3-trifluoromethylthio and the combination of (R²)ₘ, R^{z} and o corresponds in each case to a row of Table A.

Table 144

Compounds of the formula I-1 wherein A-B denotes C—C, X is oxygen, n is 1, R¹ is 4-fluoro and the combination of (R²)ₘ, R^{z} and o corresponds in each case to a row of Table A.

Table 145

Compounds of the formula I-1 wherein A-B denotes C—C, X is oxygen, n is 1, R¹ is 4-chloro and the combination of (R²)ₘ, R^{z} and o corresponds in each case to a row of Table A.

Table 146

Compounds of the formula I-1 wherein A-B denotes C—C, X is oxygen, n is 1, R¹ is 4-bromo and the combination of (R²)ₘ, R^{z} and o corresponds in each case to a row of Table A.

Table 147

Compounds of the formula I-1 wherein A-B denotes C—C, X is oxygen, n is 1, R¹ is 4-hydroxy and the combination of (R²)ₘ, R^{z} and o corresponds in each case to a row of Table A.

Table 148

Compounds of the formula I-1 wherein A-B denotes C—C, X is oxygen, n is 1, R¹ is 4-mercapto and the combination of (R²)ₘ, R^{z} and o corresponds in each case to a row of Table A.

Table 149

Compounds of the formula I-1 wherein A-B denotes C—C, X is oxygen, n is 1, R¹ is 4-cyano and the combination of (R²)ₘ, R^{z} and o corresponds in each case to a row of Table A.

Table 150

Compounds of the formula I-1 wherein A-B denotes C—C, X is oxygen, n is 1, R¹ is 4-nitro and the combination of (R²)ₘ, R^{z} and o corresponds in each case to a row of Table A.

Table 151

Compounds of the formula I-1 wherein A-B denotes C—C, X is oxygen, n is 1, R¹ is 4-methyl and the combination of (R²)ₘ, R^{z} and o corresponds in each case to a row of Table A.

Table 152

Compounds of the formula I-1 wherein A-B denotes C—C, X is oxygen, n is 1, R¹ is 4-trifluoromethyl and the combination of (R²)ₘ, R^{z} and o corresponds in each case to a row of Table A.

Table 153

Compounds of the formula I-1 wherein A-B denotes C—C, X is oxygen, n is 1, R¹ is 4-methoxy and the combination of (R²)ₘ, R^{z} and o corresponds in each case to a row of Table A.

Table 154

Compounds of the formula I-1 wherein A-B denotes C—C, X is oxygen, n is 1, R¹ is 4-trifluoromethoxy and the combination of (R²)ₘ, R^{z} and o corresponds in each case to a row of Table A.

Table 155

Compounds of the formula I-1 wherein A-B denotes C—C, X is oxygen, n is 1, R¹ is 4-trifluoromethylthio and the combination of (R²)ₘ, R^{z} and o corresponds in each case to a row of Table A.

Table 156

Compounds of the formula I-1 wherein A-B denotes C—C, X is oxygen, (R¹)ₙ is 2,3-difluoro, and the combination of (R²)ₘ, R^{z} and o corresponds in each case to a row of Table A.

Table 157

Compounds of the formula I-1 wherein A-B denotes C—C, X is oxygen, (R¹)ₙ is 2-fluoro-3-chloro, and the combination of (R²)ₘ, R^{z} and o corresponds in each case to a row of Table A.

Table 158

Compounds of the formula I-1 wherein A-B denotes C—C, X is oxygen, (R¹)ₙ is 2-chloro-3-fluoro, and the combination of (R²)ₘ, R^{z} and o corresponds in each case to a row of Table A.

Table 159

Compounds of the formula I-1 wherein A-B denotes C—C, X is oxygen, (R¹)ₙ is 2,3-dimethoxy, and the combination of (R²)ₘ, R^{z} and o corresponds in each case to a row of Table A.

Table 160

Compounds of the formula I-1 wherein A-B denotes C═C, X is sulfur, n is zero, and the combination of (R²)ₘ, R^{z} and o corresponds in each case to a row of Table A.

Table 161

Compounds of the formula I-1 wherein A-B denotes C═C, X is sulfur, n is 1, R¹ is 1-fluoro and the combination of (R²)ₘ, R^{z} and o corresponds in each case to a row of Table A.

Table 162

Compounds of the formula I-1 wherein A-B denotes C═C, X is sulfur, n is 1, R¹ is 1-chloro and the combination of (R²)ₘ, R^{z} and o corresponds in each case to a row of Table A.

Table 163

Compounds of the formula I-1 wherein A-B denotes C═C, X is sulfur, n is 1, R¹ is 1-bromo and the combination of (R²)ₘ, R^{z} and o corresponds in each case to a row of Table A.

Table 164

Compounds of the formula I-1 wherein A-B denotes C═C, X is sulfur, n is 1, R¹ is 1-hydroxy and the combination of (R²)ₘ, R^{z} and o corresponds in each case to a row of Table A.

Table 165

Compounds of the formula I-1 wherein A-B denotes C═C, X is sulfur, n is 1, R¹ is 1-mercapto and the combination of (R²)ₘ, R^{z} and o corresponds in each case to a row of Table A.

Table 166

Compounds of the formula I-1 wherein A-B denotes C═C, X is sulfur, n is 1, R¹ is 1-cyano and the combination of (R²)ₘ, R^{z} and o corresponds in each case to a row of Table A.

Table 167

Compounds of the formula I-1 wherein A-B denotes C═C, X is sulfur, n is 1, R¹ is 1-nitro and the combination of (R²)ₘ, R^{z} and o corresponds in each case to a row of Table A.

Table 168

Compounds of the formula I-1 wherein A-B denotes C═C, X is sulfur, n is 1, R¹ is 1-methyl and the combination of (R²)ₘ, R^{z} and o corresponds in each case to a row of Table A.

Table 169

Compounds of the formula I-1 wherein A-B denotes C═C, X is sulfur, n is 1, R¹ is 1-trifluoromethyl and the combination of (R²)ₘ, R^{z} and o corresponds in each case to a row of Table A.

Table 170

Compounds of the formula I-1 wherein A-B denotes C═C, X is sulfur, n is 1, R¹ is 1-methoxy and the combination of (R²)ₘ, R^{z} and o corresponds in each case to a row of Table A.

Table 171

Compounds of the formula I-1 wherein A-B denotes C═C, X is sulfur, n is 1, R¹ is 1-trifluoromethoxy and the combination of (R²)ₘ, R^{z} and o corresponds in each case to a row of Table A.

Table 172

Compounds of the formula I-1 wherein A-B denotes C═C, X is sulfur, n is 1, R¹ is 1-trifluoromethylthio and the combination of (R²)ₘ, R^{z} and o corresponds in each case to a row of Table A.

Table 173

Compounds of the formula I-1 wherein A-B denotes C═C, X is sulfur, n is 1, R¹ is 2-fluoro and the combination of (R²)ₘ, R^{z} and o corresponds in each case to a row of Table A.

Table 174

Compounds of the formula I-1 wherein A-B denotes C═C, X is sulfur, n is 1, R¹ is 2-chloro and the combination of (R²)ₘ, R^{z} and o corresponds in each case to a row of Table A.

Table 175

Compounds of the formula I-1 wherein A-B denotes C═C, X is sulfur, n is 1, R¹ is 2-bromo and the combination of (R²)ₘ, R^{z} and o corresponds in each case to a row of Table A.

Table 176

Compounds of the formula I-1 wherein A-B denotes C═C, X is sulfur, n is 1, R¹ is 2-hydroxy and the combination of (R²)ₘ, R^{z} and o corresponds in each case to a row of Table A.

Table 177

Compounds of the formula I-1 wherein A-B denotes C═C, X is sulfur, n is 1, R¹ is 2-mercapto and the combination of (R²)ₘ, R^{z} and o corresponds in each case to a row of Table A.

Table 178

Compounds of the formula I-1 wherein A-B denotes C═C, X is sulfur, n is 1, R¹ is 2-cyano and the combination of (R²)ₘ, R^{z} and o corresponds in each case to a row of Table A.

Table 179

Compounds of the formula I-1 wherein A-B denotes C═C, X is sulfur, n is 1, R¹ is 2-nitro and the combination of (R²)ₘ, R^{z} and o corresponds in each case to a row of Table A.

Table 180

Compounds of the formula I-1 wherein A-B denotes C═C, X is sulfur, n is 1, R¹ is 2-methyl and the combination of (R²)ₘ, R^{z} and o corresponds in each case to a row of Table A.

Table 181

Compounds of the formula I-1 wherein A-B denotes C═C, X is sulfur, n is 1, R¹ is 2-trifluoromethyl and the combination of (R²)ₘ, R^{z} and o corresponds in each case to a row of Table A.

Table 182

Compounds of the formula I-1 wherein A-B denotes C═C, X is sulfur, n is 1, R¹ is 2-methoxy and the combination of (R²)ₘ, R^{z} and o corresponds in each case to a row of Table A.

Table 183

Compounds of the formula I-1 wherein A-B denotes C═C, k is sulfur, n is 1, R¹ is 2-trifluoromethoxy and the combination of (R²)ₘ, R^{z} and o corresponds in each case to a row of Table A.

Table 184

Compounds of the formula I-1 wherein A-B denotes C═C, X is sulfur, n is 1, R¹ is 2-trifluoromethylthio and the combination of (R²)ₘ, R^{z} and o corresponds in each case to a row of Table A.

Table 185

Compounds of the formula I-1 wherein A-B denotes C═C, X is sulfur, n is 1, R¹ is 3-fluoro and the combination of (R²)ₘ, R^{z} and o corresponds in each case to a row of Table A.

Table 186

Compounds of the formula I-1 wherein A-B denotes C═C, X is sulfur, n is 1, R¹ is 3-chloro and the combination of (R²)ₘ, R^{z} and o corresponds in each case to a row of Table A.

Table 187

Compounds of the formula I-1 wherein A-B denotes C═C, X is sulfur, n is 1, R¹ is 3-bromo and the combination of (R²)ₘ, R^{z} and o corresponds in each case to a row of Table A.

Table 188

Compounds of the formula I-1 wherein A-B denotes C═C, X is sulfur, n is 1, R¹ is 3-hydroxy and the combination of (R²)ₘ, R^{z} and o corresponds in each case to a row of Table A.

Table 189

Compounds of the formula I-1 wherein A-B denotes C═C, X is sulfur, n is 1, R¹ is 3-mercapto and the combination of (R²)ₘ, R^{z} and o corresponds in each case to a row of Table A.

Table 190

Compounds of the formula I-1 wherein A-B denotes C═C, X is sulfur, n is 1, R¹ is 3-cyano and the combination of (R²)ₘ, R^{z} and o corresponds in each case to a row of Table A.

Table 191

Compounds of the formula I-1 wherein A-B denotes C═C, X is sulfur, n is 1, R¹ is 3-nitro and the combination of (R²)ₘ, R^{z} and o corresponds in each case to a row of Table A.

Table 192

Compounds of the formula I-1 wherein A-B denotes C═C, X is sulfur, n is 1, R¹ is 3-methyl and the combination of (R²)ₘ, R^{z} and o corresponds in each case to a row of Table A.

Table 193

Compounds of the formula I-1 wherein A-B denotes C═C, X is sulfur, n is 1, R¹ is 3-trifluoromethyl and the combination of (R²)ₘ, R^{z} and o corresponds in each case to a row of Table A.

Table 194

Compounds of the formula I-1 wherein A-B denotes C═C, X is sulfur, n is 1, R¹ is 3-methoxy and the combination of (R²)ₘ, R^{z} and o corresponds in each case to a row of Table A.

Table 195

Compounds of the formula I-1 wherein A-B denotes C═C, X is sulfur, n is 1, R¹ is 3-trifluoromethoxy and the combination of (R²)ₘ, R^{z} and o corresponds in each case to a row of Table A.

Table 196

Compounds of the formula I-1 wherein A-B denotes C═C, X is sulfur, n is 1, R¹ is 3-trifluoromethylthio and the combination of (R²)ₘ, R^{z} and o corresponds in each case to a row of Table A.

Table 197

Compounds of the formula I-1 wherein A-B denotes C═C, X is sulfur, n is 1, R¹ is 4-fluoro and the combination of (R²)ₘ, R^{z} and o corresponds in each case to a row of Table A.

Table 198

Compounds of the formula I-1 wherein A-B denotes C═C, X is sulfur, n is 1, R¹ is 4-chloro and the combination of (R²)ₘ, R^{z} and o corresponds in each case to a row of Table A.

Table 199

Compounds of the formula I-1 wherein A-B denotes C═C, X is sulfur, n is 1, R¹ is 4-bromo and the combination of (R²)ₘ, R^{z} and o corresponds in each case to a row of Table A.

Table 200

Compounds of the formula I-1 wherein A-B denotes C═C, X is sulfur, n is 1, R¹ is 4-hydroxy and the combination of (R²)ₘ, R^{z} and o corresponds in each case to a row of Table A.

Table 201

Compounds of the formula I-1 wherein A-B denotes C═C, X is sulfur, n is 1, R¹ is 4-mercapto and the combination of (R²)ₘ, R^{z} and o corresponds in each case to a row of Table A.

Table 202

Compounds of the formula I-1 wherein A-B denotes C═C, X is sulfur, n is 1, R¹ is 4-cyano and the combination of (R²)ₘ, R^{z} and o corresponds in each case to a row of Table A.

Table 203

Compounds of the formula I-1 wherein A-B denotes C═C, X is sulfur, n is 1, R¹ is 4-nitro and the combination of (R²)ₘ, R^{z} and o corresponds in each case to a row of Table A.

Table 204

Compounds of the formula I-1 wherein A-B denotes C═C, X is sulfur, n is 1, R¹ is 4-methyl and the combination of (R²)ₘ, R^{z} and o corresponds in each case to a row of Table A.

Table 205

Compounds of the formula I-1 wherein A-B denotes C═C, X is sulfur, n is 1, R¹ is 4-trifluoromethyl and the combination of (R²)ₘ, R^{z} and o corresponds in each case to a row of Table A.

Table 206

Compounds of the formula I-1 wherein A-B denotes C═C, X is sulfur, n is 1, R¹ is 4-methoxy and the combination of (R²)ₘ, R^{z} and o corresponds in each case to a row of Table A.

Table 207

Compounds of the formula I-1 wherein A-B denotes C═C, X is sulfur, n is 1, R¹ is 4-trifluoromethoxy and the combination of (R²)ₘ, R^{z} and o corresponds in each case to a row of Table A.

Table 208

Compounds of the formula I-1 wherein A-B denotes C═C, X is sulfur, n is 1, R¹ is 4-trifluoromethylthio and the combination of (R²)ₘ, R^{z} and o corresponds in each case to a row of Table A.

Table 209

Compounds of the formula I-1 wherein A-B denotes C═C, X is sulfur, (R¹)ₙ is 2,3-difluoro, and the combination of (R²)ₘ, R^{z} and o corresponds in each case to a row of Table A.

Table 210

Compounds of the formula I-1 wherein A-B denotes C═C, X is sulfur, (R¹)ₙ is 2-fluoro-3-chloro, and the combination of (R²)ₘ, R^{z} and o corresponds in each case to a row of Table A.

Table 211

Compounds of the formula I-1 wherein A-B denotes C═C, X is sulfur, (R¹)ₙ is 2-chloro-3-fluoro, and the combination of (R²)ₘ, R^{z} and o corresponds in each case to a row of Table A.

Table 212

Compounds of the formula I-1 wherein A-B denotes C═C, X is sulfur, (R¹)ₙ is 2,3-dimethoxy, and the combination of (R²)ₘ, R^{z} and o corresponds in each case to a row of Table A.

Table 213

Compounds of the formula I-1 wherein A-B denotes C═C, X is methylene, n is zero, and the combination of (R²)ₘ, R^{z} and o corresponds in each case to a row of Table A.

Table 214

Compounds of the formula I-1 wherein A-B denotes C═C, X is methylene, n is 1, R¹ is 1-fluoro and the combination of (R²)ₘ, R^{z} and o corresponds in each case to a row of Table A.

Table 215

Compounds of the formula I-1 wherein A-B denotes C═C, X is methylene, n is 1, R¹ is 1-chloro and the combination of (R²)ₘ, R^{z} and o corresponds in each case to a row of Table A.

Table 216

Compounds of the formula I-1 wherein A-B denotes C═C, X is methylene, n is 1, R¹ is 1-bromo and the combination of (R²)ₘ, R^{z} and o corresponds in each case to a row of Table A.

Table 217

Compounds of the formula I-1 wherein A-B denotes C═C, X is methylene, n is 1, R¹ is 1-hydroxy and the combination of (R²)ₘ, R^{z} and o corresponds in each case to a row of Table A.

Table 218

Compounds of the formula I-1 wherein A-B denotes C═C, X is methylene, n is 1, R¹ is 1-mercapto and the combination of (R²)ₘ, R^{z} and o corresponds in each case to a row of Table A.

Table 219

Compounds of the formula I-1 wherein A-B denotes C═C, X is methylene, n is 1, R¹ is 1-cyano and the combination of (R²)ₘ, R^{z} and o corresponds in each case to a row of Table A.

Table 220

Compounds of the formula I-1 wherein A-B denotes C═C, X is methylene, n is 1, R¹ is 1-nitro and the combination of (R²)ₘ, R^{z} and o corresponds in each case to a row of Table A.

Table 221

Compounds of the formula I-1 wherein A-B denotes C═C, X is methylene, n is 1, R¹ is 1-methyl and the combination of (R²)ₘ, R^{z} and o corresponds in each case to a row of Table A.

Table 222

Compounds of the formula I-1 wherein A-B denotes C═C, X is methylene, n is 1, R¹ is 1-trifluoromethyl and the combination of (R²)ₘ, R^{z} and o corresponds in each case to a row of Table A.

Table 223

Compounds of the formula I-1 wherein A-B denotes C═C, X is methylene, n is 1, R¹ is 1-methoxy and the combination of (R²)ₘ, R^{z} and o corresponds in each case to a row of Table A.

Table 224

Compounds of the formula I-1 wherein A-B denotes C═C, X is methylene, n is 1, R¹ is 1-trifluoromethoxy and the combination of (R²)ₘ, R^{z} and o corresponds in each case to a row of Table A.

Table 225

Compounds of the formula I-1 wherein A-B denotes C═C, X is methylene, n is 1, R¹ is 1-trifluoromethylthio and the combination of (R²)ₘ, R^{z} and o corresponds in each case to a row of Table A.

Table 226

Compounds of the formula I-1 wherein A-B denotes C═C, X is methylene, n is 1, R¹ is 2-fluoro and the combination of (R²)ₘ, R^{z} and o corresponds in each case to a row of Table A.

Table 227

Compounds of the formula I-1 wherein A-B denotes C═C, X is methylene, n is 1, R¹ is 2-chloro and the combination of (R²)ₘ, R^{z} and o corresponds in each case to a row of Table A.

Table 228

Compounds of the formula I-1 wherein A-B denotes C═C, X is methylene, n is 1, R¹ is 2-bromo and the combination of (R²)ₘ, R^{z} and o corresponds in each case to a row of Table A.

Table 229

Compounds of the formula I-1 wherein A-B denotes C═C, X is methylene, n is 1, R¹ is 2-hydroxy and the combination of (R²)ₘ, R^{z} and o corresponds in each case to a row of Table A.

Table 230

Compounds of the formula I-1 wherein A-B denotes C═C, X is methylene, n is 1, R¹ is 2-mercapto and the combination of (R²)ₘ, R^{z} and o corresponds in each case to a row of Table A.

Table 231

Compounds of the formula I-1 wherein A-B denotes C═C, X is methylene, n is 1, R¹ is 2-cyano and the combination of (R²)ₘ, R^{z} and o corresponds in each case to a row of Table A.

Table 232

Compounds of the formula I-1 wherein A-B denotes C═C, X is methylene, n is 1, R¹ is 2-nitro and the combination of (R²)ₘ, R^{z} and o corresponds in each case to a row of Table A.

Table 233

Compounds of the formula I-1 wherein A-B denotes C═C, X is methylene, n is 1, R¹ is 2-methyl and the combination of (R²)ₘ, R^{z} and o corresponds in each case to a row of Table A.

Table 234

Compounds of the formula I-1 wherein A-B denotes C═C, X is methylene, n is 1, R¹ is 2-trifluoromethyl and the combination of (R²)ₘ, R^{z} and o corresponds in each case to a row of Table A.

Table 235

Compounds of the formula I-1 wherein A-B denotes C═C, X is methylene, n is 1, R¹ is 2-methoxy and the combination of (R²)ₘ, R^{z} and o corresponds in each case to a row of Table A.

Table 236

Compounds of the formula I-1 wherein A-B denotes C═C, X is methylene, n is 1, R¹ is 2-trifluoromethoxy and the combination of (R²)ₘ, R^{z} and o corresponds in each case to a row of Table A.

Table 237

Compounds of the formula I-1 wherein A-B denotes C═C, X is methylene, n is 1, R¹ is 2-trifluoromethylthio and the combination of (R²)ₘ, R^{z} and o corresponds in each case to a row of Table A.

Table 238

Compounds of the formula I-1 wherein A-B denotes C═C, X is methylene, n is 1, R¹ is 3-fluoro and the combination of (R²)ₘ, R^{z} and o corresponds in each case to a row of Table A.

Table 239

Compounds of the formula I-1 wherein A-B denotes C═C, X is methylene, n is 1, R¹ is 3-chloro and the combination of (R²)ₘ, R^{z} and o corresponds in each case to a row of Table A.

Table 240

Compounds of the formula I-1 wherein A-B denotes C═C, X is methylene, n is 1, R¹ is 3-bromo and the combination of (R²)ₘ, R^{z} and o corresponds in each case to a row of Table A.

Table 241

Compounds of the formula I-1 wherein A-B denotes C═C, X is methylene, n is 1, R¹ is 3-hydroxy and the combination of (R²)ₘ, R^{z} and o corresponds in each case to a row of Table A.

Table 242

Compounds of the formula I-1 wherein A-B denotes C═C, X is methylene, n is 1, R¹ is 3-mercapto and the combination of (R²)ₘ, R^{z} and o corresponds in each case to a row of Table A.

Table 243

Compounds of the formula I-1 wherein A-B denotes C═C, X is methylene, n is 1, R¹ is 3-cyano and the combination of (R²)ₘ, R^{z} and o corresponds in each case to a row of Table A.

Table 244

Compounds of the formula I-1 wherein A-B denotes C═C, X is methylene, n is 1, R¹ is 3-nitro and the combination of (R²)ₘ, R^{z} and o corresponds in each case to a row of Table A.

Table 245

Compounds of the formula I-1 wherein A-B denotes C═C, X is methylene, n is 1, R¹ is 3-methyl and the combination of (R²)ₘ, R^{z} and o corresponds in each case to a row of Table A.

Table 246

Compounds of the formula I-1 wherein A-B denotes C═C, X is methylene, n is 1, R¹ is 3-trifluoromethyl and the combination of (R²)ₘ, R^{z} and o corresponds in each case to a row of Table A.

Table 247

Compounds of the formula I-1 wherein A-B denotes C═C, X is methylene, n is 1, R¹ is 3-methoxy and the combination of (R²)ₘ, R^{z} and o corresponds in each case to a row of Table A.

Table 248

Compounds of the formula I-1 wherein A-B denotes C═C, X is methylene, n is 1, R¹ is 3-trifluoromethoxy and the combination of (R²)ₘ, R^{z} and o corresponds in each case to a row of Table A.

Table 249

Compounds of the formula I-1 wherein A-B denotes C═C, X is methylene, n is 1, R¹ is 3-trifluoromethylthio and the combination of (R²)ₘ, R^{z} and o corresponds in each case to a row of Table A.

Table 250

Compounds of the formula I-1 wherein A-B denotes C═C, X is methylene, n is 1, R¹ is 4-fluoro and the combination of (R²)ₘ, R^{z} and o corresponds in each case to a row of Table A.

Table 251

Compounds of the formula I-1 wherein A-B denotes C═C, X is methylene, n is 1, R¹ is 4-chloro and the combination of (R²)ₘ, R^{z} and o corresponds in each case to a row of Table A.

Table 252

Compounds of the formula I-1 wherein A-B denotes C═C, X is methylene, n is 1, R¹ is 4-bromo and the combination of (R²)ₘ, R^{z} and o corresponds in each case to a row of Table A.

Table 253

Compounds of the formula I-1 wherein A-B denotes C═C, X is methylene, n is 1, R¹ is 4-hydroxy and the combination of (R²)ₘ, R^{z} and o corresponds in each case to a row of Table A.

Table 254

Compounds of the formula I-1 wherein A-B denotes C═C, X is methylene, n is 1, R¹ is 4-mercapto and the combination of (R²)ₘ, R^{z} and o corresponds in each case to a row of Table A.

Table 255

Compounds of the formula I-1 wherein A-B denotes C═C, X is methylene, n is 1, R¹ is 4-cyano and the combination of (R²)ₘ, R^{z} and o corresponds in each case to a row of Table A.

Table 256

Compounds of the formula I-1 wherein A-B denotes C═C, X is methylene, n is 1, R¹ is 4-nitro and the combination of (R²)ₘ, R^{z} and o corresponds in each case to a row of Table A.

Table 257

Compounds of the formula I-1 wherein A-B denotes C═C, X is methylene, n is 1, R¹ is 4-methyl and the combination of (R²)ₘ, R^{z} and o corresponds in each case to a row of Table A.

Table 258

Compounds of the formula I-1 wherein A-B denotes C═C, X is methylene, n is 1, R¹ is 4-trifluoromethyl and the combination of (R²)ₘ, R^{z} and o corresponds in each case to a row of Table A.

Table 259

Compounds of the formula I-1 wherein A-B denotes C═C, X is methylene, n is 1, R¹ is 4-methoxy and the combination of (R²)ₘ, R^{z} and o corresponds in each case to a row of Table A.

Table 260

Compounds of the formula I-1 wherein A-B denotes C═C, X is methylene, n is 1, R¹ is 4-trifluoromethoxy and the combination of (R²)ₘ, R^{z} and o corresponds in each case to a row of Table A.

Table 261

Compounds of the formula I-1 wherein A-B denotes C═C, X is methylene, n is 1, R¹ is 4-trifluoromethylthio and the combination of (R²)ₘ, R^{z} and o corresponds in each case to a row of Table A.

Table 262

Compounds of the formula I-1 wherein A-B denotes C═C, X is methylene, (R¹)ₙ is 2,3-difluoro, and the combination of (R²)ₘ, R^{z} and o corresponds in each case to a row of Table A.

Table 263

Compounds of the formula I-1 wherein A-B denotes C═C, X is methylene, (R¹)ₙ is 2-fluoro-3-chloro, and the combination of (R²)ₘ, R^{z} and o corresponds in each case to a row of Table A.

Table 264

Compounds of the formula I-1 wherein A-B denotes C═C, X is methylene, (R¹)ₙ is 2-chloro-3-fluoro, and the combination of (R²)ₘ, R^{z} and o corresponds in each case to a row of Table A.

Table 265

Compounds of the formula I-1 wherein A-B denotes C═C, X is methylene, (R¹)ₙ is 2,3-dimethoxy, and the combination of (R²)ₘ, R^{z} and o corresponds in each case to a row of Table A.

Table 266

Compounds of the formula I-1 wherein A-B denotes C═C, X is oxygen, n is zero, and the combination of (R²)ₘ, R^{z} and o corresponds in each case to a row of Table A.

Table 267

Compounds of the formula I-1 wherein A-B denotes C═C, X is oxygen, n is 1, R¹ is 1-fluoro and the combination of (R²)ₘ, R^{z} and o corresponds in each case to a row of Table A.

Table 268

Compounds of the formula I-1 wherein A-B denotes C═C, X is oxygen, n is 1, R¹ is 1-chloro and the combination of (R²)ₘ, R^{z} and o corresponds in each case to a row of Table A.

Table 269

Compounds of the formula I-1 wherein A-B denotes C═C, X is oxygen, n is 1, R¹ is 1-bromo and the combination of (R²)ₘ, R^{z} and o corresponds in each case to a row of Table A.

Table 270

Compounds of the formula I-1 wherein A-B denotes C═C, X is oxygen, n is 1, R¹ is 1-hydroxy and the combination of (R²)ₘ, R^{z} and o corresponds in each case to a row of Table A.

Table 271

Compounds of the formula I-1 wherein A-B denotes C═C, X is oxygen, n is 1, R¹ is 1-mercapto and the combination of (R²)ₘ, R^{z} and o corresponds in each case to a row of Table A.

Table 272

Compounds of the formula I-1 wherein A-B denotes C═C, X is oxygen, n is 1, R¹ is 1-cyano and the combination of (R²)ₘ, R^{z} and o corresponds in each case to a row of Table A.

Table 273

Compounds of the formula I-1 wherein A-B denotes C═C, X is oxygen, n is 1, R¹ is 1-nitro and the combination of (R²)ₘ, R^{z} and o corresponds in each case to a row of Table A.

Table 274

Compounds of the formula I-1 wherein A-B denotes C═C, X is oxygen, n is 1, R¹ is 1-methyl and the combination of (R²)ₘ, R^{z} and o corresponds in each case to a row of Table A.

Table 275

Compounds of the formula I-1 wherein A-B denotes C═C, X is oxygen, n is 1, R¹ is 1-trifluoromethyl and the combination of (R²)ₘ, R^{z} and o corresponds in each case to a row of Table A.

Table 276

Compounds of the formula I-1 wherein A-B denotes C═C, X is oxygen, n is 1, R¹ is 1-methoxy and the combination of (R²)ₘ, R^{z} and o corresponds in each case to a row of Table A.

Table 277

Compounds of the formula I-1 wherein A-B denotes C═C, X is oxygen, n is 1, R¹ is 1-trifluoromethoxy and the combination of (R²)ₘ, R^{z} and o corresponds in each case to a row of Table A.

Table 278

Compounds of the formula I-1 wherein A-B denotes C═C, X is oxygen, n is 1, R¹ is 1-trifluoromethylthio and the combination of (R²)ₘ, R^{z} and o corresponds in each case to a row of Table A.

Table 279

Compounds of the formula I-1 wherein A-B denotes C═C, X is oxygen, n is 1, R¹ is 2-fluoro and the combination of (R²)ₘ, R^{z} and o corresponds in each case to a row of Table A.

Table 280

Compounds of the formula I-1 wherein A-B denotes C═C, X is oxygen, n is 1, R¹ is 2-chloro and the combination of (R²)ₘ, R^{z} and o corresponds in each case to a row of Table A.

Table 281

Compounds of the formula I-1 wherein A-B denotes C═C, X is oxygen, n is 1, R¹ is 2-bromo and the combination of (R²)ₘ, R^{z} and o corresponds in each case to a row of Table A.

Table 282

Compounds of the formula I-1 wherein A-B denotes C═C, X is oxygen, n is 1, R¹ is 2-hydroxy and the combination of (R²)ₘ, R^{z} and o corresponds in each case to a row of Table A.

Table 283

Compounds of the formula I-1 wherein A-B denotes C═C, X is oxygen, n is 1, R¹ is 2-mercapto and the combination of (R²)ₘ, R^{z} and o corresponds in each case to a row of Table A.

Table 284

Compounds of the formula I-1 wherein A-B denotes C═C, X is oxygen, n is 1, R¹ is 2-cyano and the combination of (R²)ₘ, R^{z} and o corresponds in each case to a row of Table A.

Table 285

Compounds of the formula I-1 wherein A-B denotes C═C, X is oxygen, n is 1, R¹ is 2-nitro and the combination of (R²)ₘ, R^{z} and o corresponds in each case to a row of Table A.

Table 286

Compounds of the formula I-1 wherein A-B denotes C═C, X is oxygen, n is 1, R¹ is 2-methyl and the combination of (R²)ₘ, R^{z} and o corresponds in each case to a row of Table A.

Table 287

Compounds of the formula I-1 wherein A-B denotes C═C, X is oxygen, n is 1, R¹ is 2-trifluoromethyl and the combination of (R²)ₘ, R^{z} and o corresponds in each case to a row of Table A.

Table 288

Compounds of the formula I-1 wherein A-B denotes C═C, X is oxygen, n is 1, R¹ is 2-methoxy and the combination of (R²)ₘ, R^{z} and o corresponds in each case to a row of Table A.

Table 289

Compounds of the formula I-1 wherein A-B denotes C═C, X is oxygen, n is 1, R¹ is 2-trifluoromethoxy and the combination of (R²)ₘ, R^{z} and o corresponds in each case to a row of Table A.

Table 290

Compounds of the formula I-1 wherein A-B denotes C═C, X is oxygen, n is 1, R¹ is 2-trifluoromethylthio and the combination of (R²)ₘ, R^{z} and o corresponds in each case to a row of Table A.

Table 291

Compounds of the formula I-1 wherein A-B denotes C═C, X is oxygen, n is 1, R¹ is 3-fluoro and the combination of (R²)ₘ, R^{z} and o corresponds in each case to a row of Table A.

Table 292

Compounds of the formula I-1 wherein A-B denotes C═C, X is oxygen, n is 1, R¹ is 3-chloro and the combination of (R²)ₘ, R^{z} and o corresponds in each case to a row of Table A.

Table 293

Compounds of the formula I-1 wherein A-B denotes C═C, X is oxygen, n is 1, R¹ is 3-bromo and the combination of (R²)ₘ, R^{z} and o corresponds in each case to a row of Table A.

Table 294

Compounds of the formula I-1 wherein A-B denotes C═C, X is oxygen, n is 1, R¹ is 3-hydroxy and the combination of (R²)ₘ, R^{z} and o corresponds in each case to a row of Table A.

Table 295

Compounds of the formula I-1 wherein A-B denotes C═C, X is oxygen, n is 1, R¹ is 3-mercapto and the combination of (R²)ₘ, R^{z} and o corresponds in each case to a row of Table A.

Table 296

Compounds of the formula I-1 wherein A-B denotes C═C, X is oxygen, n is 1, R¹ is 3-cyano and the combination of (R²)ₘ, R^{z} and o corresponds in each case to a row of Table A.

Table 297

Compounds of the formula I-1 wherein A-B denotes C═C, X is oxygen, n is 1, R¹ is 3-nitro and the combination of (R²)ₘ, R^{z} and o corresponds in each case to a row of Table A.

Table 298

Compounds of the formula I-1 wherein A-B denotes C═C, X is oxygen, n is 1, R¹ is 3-methyl and the combination of (R²)ₘ, R^{z} and o corresponds in each case to a row of Table A.

Table 299

Compounds of the formula I-1 wherein A-B denotes C═C, X is oxygen, n is 1, R¹ is 3-trifluoromethyl and the combination of (R²)ₘ, R^{z} and o corresponds in each case to a row of Table A.

Table 300

Compounds of the formula I-1 wherein A-B denotes C═C, X is oxygen, n is 1, R¹ is 3-methoxy and the combination of (R²)ₘ, R^{z} and o corresponds in each case to a row of Table A.

Table 301

Compounds of the formula I-1 wherein A-B denotes C═C, X is oxygen, n is 1, R¹ is 3-trifluoromethoxy and the combination of (R²)ₘ, R^{z} and o corresponds in each case to a row of Table A.

Table 302

Compounds of the formula I-1 wherein A-B denotes C═C, X is oxygen, n is 1, R¹ is 3-trifluoromethylthio and the combination of (R²)ₘ, R^{z} and o corresponds in each case to a row of Table A.

Table 303

Compounds of the formula I-1 wherein A-B denotes C═C, X is oxygen, n is 1, R¹ is 4-fluoro and the combination of (R²)ₘ, R^{z} and o corresponds in each case to a row of Table A.

Table 304

Compounds of the formula I-1 wherein A-B denotes C═C, X is oxygen, n is 1, R¹ is 4-chloro and the combination of (R²)ₘ, R^{z} and o corresponds in each case to a row of Table A.

Table 305

Compounds of the formula I-1 wherein A-B denotes C═C, X is oxygen, n is 1, R¹ is 4-bromo and the combination of (R²)ₘ, R^{z} and o corresponds in each case to a row of Table A.

Table 306

Compounds of the formula I-1 wherein A-B denotes C═C, X is oxygen, n is 1, R¹ is 4-hydroxy and the combination of (R²)ₘ, R^{z} and o corresponds in each case to a row of Table A.

Table 307

Compounds of the formula I-1 wherein A-B denotes C═C, X is oxygen, n is 1, R¹ is 4-mercapto and the combination of (R²)ₘ, R^{z} and o corresponds in each case to a row of Table A.

Table 308

Compounds of the formula I-1 wherein A-B denotes C═C, X is oxygen, n is 1, R¹ is 4-cyano and the combination of (R²)ₘ, R^{z} and o corresponds in each case to a row of Table A.

Table 309

Compounds of the formula I-1 wherein A-B denotes C═C, X is oxygen, n is 1, R¹ is 4-nitro and the combination of (R²)ₘ, R^{z} and o corresponds in each case to a row of Table A.

Table 310

Compounds of the formula I-1 wherein A-B denotes C═C, X is oxygen, n is 1, R¹ is 4-methyl and the combination of (R²)ₘ, R^{z} and o corresponds in each case to a row of Table A.

Table 311

Compounds of the formula I-1 wherein A-B denotes C═C, X is oxygen, n is 1, R¹ is 4-trifluoromethyl and the combination of (R²)ₘ, R^{z} and o corresponds in each case to a row of Table A.

Table 312

Compounds of the formula I-1 wherein A-B denotes C═C, X is oxygen, n is 1, R¹ is 4-methoxy and the combination of (R²)ₘ, R^{z} and o corresponds in each case to a row of Table A.

Table 313

Compounds of the formula I-1 wherein A-B denotes C═C, X is oxygen, n is 1, R¹ is 4-trifluoromethoxy and the combination of (R²)ₘ, R^{z} and o corresponds in each case to a row of Table A.

Table 314

Compounds of the formula I-1 wherein A-B denotes C═C, X is oxygen, n is 1, R¹ is 4-trifluoromethylthio and the combination of (R²)ₘ, R^{z} and o corresponds in each case to a row of Table A.

Table 315

Compounds of the formula I-1 wherein A-B denotes C═C, X is oxygen, (R¹)ₙ is 2,3-difluoro, and the combination of (R²)ₘ, R^{z} and o corresponds in each case to a row of Table A.

Table 316

Compounds of the formula I-1 wherein A-B denotes C═C, X is oxygen, (R¹)ₙ is 2-fluoro-3-chloro, and the combination of (R²)ₘ, R^{z} and o corresponds in each case to a row of Table A.

Table 317

Compounds of the formula I-1 wherein A-B denotes C═C, X is oxygen, (R¹)ₙ is 2-chloro-3-fluoro, and the combination of (R²)ₘ, R^{z} and o corresponds in each case to a row of Table A.

Table 318

Compounds of the formula I-1 wherein A-B denotes C═C, X is oxygen, (R¹)ₙ is 2,3-dimethoxy, and the combination of (R²)ₘ, R^{z} and o corresponds in each case to a row of Table A.

Table 319

Compounds of the formula I-1 wherein A-B denotes C—C, X is NH, n is zero, and the combination of (R²)ₘ, R^{z} and o corresponds in each case to a row of Table A.

Table 320

Compounds of the formula I-1 wherein A-B denotes C—C, X is NH, n is 1, R¹ is 2-chloro and the combination of (R²)ₘ, R^{z} and o corresponds in each case to a row of Table A.

Table 321

Compounds of the formula I-1 wherein A-B denotes C—C, X is NH, n is 1, R¹ is 2-methyl and the combination of (R²)ₘ, R^{z} and o corresponds in each case to a row of Table A.

Table 322

Compounds of the formula I-1 wherein A-B denotes C—C, X is NH, n is 1, R¹ is 2-trifluoromethyl and the combination of (R²)ₘ, R^{z} and o corresponds in each case to a row of Table A.

Table 323

Compounds of the formula I-1 wherein A-B denotes C═C, X is NH, n is zero, and the combination of (R²)ₘ, R^{z} and o corresponds in each case to a row of Table A.

Table 324

Compounds of the formula I-1 wherein A-B denotes C═C, X is NH, n is 1, R¹ is 2-chloro and the combination of (R²)ₘ, R^{z} and o corresponds in each case to a row of Table A.

Table 325

Compounds of the formula I-1 wherein A-B denotes C═C, X is NH, n is 1, R¹ is 2-methyl and the combination of (R²)ₘ, R^{z} and o corresponds in each case to a row of Table A.

Table 326

Compounds of the formula I-1 wherein A-B denotes C═C, X is NH, n is 1, R¹ is 2-trifluoromethyl and the combination of (R²)ₘ, R^{z} and o corresponds in each case to a row of Table A.

TABLE A

| Nr. | (R²)ₘ | R^{z} | o |
|---|---|---|---|
| I-1-1 | — | CH₃ | 1 |
| I-1-2 | — | CH₃ | 2 |
| I-1-3 | — | CH₂CH₃ | 1 |
| I-1-4 | — | CH₂CH₃ | 2 |
| I-1-5 | — | (CH₂)₂CH₃ | 1 |
| I-1-6 | — | CH(CH₃)₂ | 1 |
| I-1-7 | — | CH₂CHCH₂ | 1 |
| I-1-8 | — | CH₂CCH | 1 |
| I-1-9 | — | cyclo-C₃H₅ | 1 |
| I-1-10 | — | CH₂-cyclo-C₃H₅ | 1 |
| I-1-11 | — | C(CH₃)₃ | 1 |
| I-1-12 | — | CH₂CF₃ | 1 |
| I-1-13 | 6-OH | CH₃ | 1 |
| I-1-14 | 6-OH | CH₃ | 2 |
| I-1-15 | 6-OH | CH₂CH₃ | 1 |
| I-1-16 | 6-OH | CH₂CH₃ | 2 |
| I-1-17 | 6-OH | (CH₂)₂CH₃ | 1 |
| I-1-18 | 6-OH | CH(CH₃)₂ | 1 |
| I-1-19 | 6-OH | CH₂CHCH₂ | 1 |
| I-1-20 | 6-OH | CH₂CCH | 1 |
| I-1-21 | 6-OH | cyclo-C₃H₅ | 1 |
| I-1-22 | 6-OH | CH₂-cyclo-C₃H₅ | 1 |
| I-1-23 | 6-OH | C(CH₃)₃ | 1 |
| I-1-24 | 6-OH | CH₂CF₃ | 1 |
| I-1-25 | 7-OH | CH₃ | 1 |
| I-1-26 | 7-OH | CH₃ | 2 |
| I-1-27 | 7-OH | CH₂CH₃ | 1 |
| I-1-28 | 7-OH | CH₂CH₃ | 2 |
| I-1-29 | 7-OH | (CH₂)₂CH₃ | 1 |
| I-1-30 | 7-OH | CH(CH₃)₂ | 1 |
| I-1-31 | 7-OH | CH₂CHCH₂ | 1 |
| I-1-32 | 7-OH | CH₂CCH | 1 |
| I-1-33 | 7-OH | cyclo-C₃H₅ | 1 |
| I-1-34 | 7-OH | CH₂-cyclo-C₃H₅ | 1 |
| I-1-35 | 7-OH | C(CH₃)₃ | 1 |
| I-1-36 | 7-OH | CH₂CF₃ | 1 |
| I-1-37 | 8-OH | CH₃ | 1 |
| I-1-38 | 8-OH | CH₃ | 2 |
| I-1-39 | 8-OH | CH₂CH₃ | 1 |
| I-1-40 | 8-OH | CH₂CH₃ | 2 |
| I-1-41 | 8-OH | (CH₂)₂CH₃ | 1 |
| I-1-42 | 8-OH | CH(CH₃)₂ | 1 |
| I-1-43 | 8-OH | CH₂CHCH₂ | 1 |
| I-1-44 | 8-OH | CH₂CCH | 1 |
| I-1-45 | 8-OH | cyclo-C₃H₅ | 1 |
| I-1-46 | 8-OH | CH₂-cyclo-C₃H₅ | 1 |
| I-1-47 | 8-OH | C(CH₃)₃ | 1 |
| I-1-48 | 8-OH | CH₂CF₃ | 1 |
| I-1-49 | 6-SH | CH₃ | 1 |
| I-1-50 | 6-SH | CH₃ | 2 |
| I-1-51 | 6-SH | CH₂CH₃ | 1 |
| I-1-52 | 6-SH | CH₂CH₃ | 2 |
| I-1-53 | 6-SH | (CH₂)₂CH₃ | 1 |
| I-1-54 | 6-SH | CH(CH₃)₂ | 1 |
| I-1-55 | 6-SH | CH₂CHCH₂ | 1 |
| I-1-56 | 6-SH | CH₂CCH | 1 |
| I-1-57 | 6-SH | cyclo-C₃H₅ | 1 |
| I-1-58 | 6-SH | CH₂-cyclo-C₃H₅ | 1 |
| I-1-59 | 6-SH | C(CH₃)₃ | 1 |
| I-1-60 | 6-SH | CH₂CF₃ | 1 |
| I-1-61 | 7-SH | CH₃ | 1 |
| I-1-62 | 7-SH | CH₃ | 2 |
| I-1-63 | 7-SH | CH₂CH₃ | 1 |
| I-1-64 | 7-SH | CH₂CH₃ | 2 |
| I-1-65 | 7-SH | (CH₂)₂CH₃ | 1 |
| I-1-66 | 7-SH | CH(CH₃)₂ | 1 |
| I-1-67 | 7-SH | CH₂CHCH₂ | 1 |
| I-1-68 | 7-SH | CH₂CCH | 1 |
| I-1-69 | 7-SH | cyclo-C₃H₅ | 1 |
| I-1-70 | 7-SH | CH₂-cyclo-C₃H₅ | 1 |
| I-1-71 | 7-SH | C(CH₃)₃ | 1 |
| I-1-72 | 7-SH | CH₂CF₃ | 1 |
| I-1-73 | 8-SH | CH₃ | 1 |
| I-1-74 | 8-SH | CH₃ | 2 |
| I-1-75 | 8-SH | CH₂CH₃ | 1 |
| I-1-76 | 8-SH | CH₂CH₃ | 2 |
| I-1-77 | 8-SH | (CH₂)₂CH₃ | 1 |
| I-1-78 | 8-SH | CH(CH₃)₂ | 1 |
| I-1-79 | 8-SH | CH₂CHCH₂ | 1 |
| I-1-80 | 8-SH | CH₂CCH | 1 |
| I-1-81 | 8-SH | cyclo-C₃H₅ | 1 |
| I-1-82 | 8-SH | CH₂-cyclo-C₃H₅ | 1 |
| I-1-83 | 8-SH | C(CH₃)₃ | 1 |
| I-1-84 | 8-SH | CH₂CF₃ | 1 |
| I-1-85 | 6-F | CH₃ | 1 |
| I-1-86 | 6-F | CH₃ | 2 |
| I-1-87 | 6-F | CH₂CH₃ | 1 |
| I-1-88 | 6-F | CH₂CH₃ | 2 |
| I-1-89 | 6-F | (CH₂)₂CH₃ | 1 |
| I-1-90 | 6-F | CH(CH₃)₂ | 1 |
| I-1-91 | 6-F | CH₂CHCH₂ | 1 |
| I-1-92 | 6-F | CH₂CCH | 1 |
| I-1-93 | 6-F | cyclo-C₃H₅ | 1 |
| I-1-94 | 6-F | CH₂-cyclo-C₃H₅ | 1 |
| I-1-95 | 6-F | C(CH₃)₃ | 1 |
| I-1-96 | 6-F | CH₂CF₃ | 1 |
| I-1-97 | 7-F | CH₃ | 1 |
| I-1-98 | 7-F | CH₃ | 2 |
| I-1-99 | 7-F | CH₂CH₃ | 1 |
| I-1-100 | 7-F | CH₂CH₃ | 2 |
| I-1-101 | 7-F | (CH₂)₂CH₃ | 1 |
| I-1-102 | 7-F | CH(CH₃)₂ | 1 |
| I-1-103 | 7-F | CH₂CHCH₂ | 1 |
| I-1-104 | 7-F | CH₂CCH | 1 |
| I-1-105 | 7-F | cyclo-C₃H₅ | 1 |
| I-1-106 | 7-F | CH₂-cyclo-C₃H₅ | 1 |
| I-1-107 | 7-F | C(CH₃)₃ | 1 |
| I-1-108 | 7-F | CH₂CF₃ | 1 |
| I-1-109 | 8-F | CH₃ | 1 |
| I-1-110 | 8-F | CH₃ | 2 |
| I-1-111 | 8-F | CH₂CH₃ | 1 |
| I-1-112 | 8-F | CH₂CH₃ | 2 |
| I-1-113 | 8-F | (CH₂)₂CH₃ | 1 |
| I-1-114 | 8-F | CH(CH₃)₂ | 1 |
| I-1-115 | 8-F | CH₂CHCH₂ | 1 |
| I-1-116 | 8-F | CH₂CCH | 1 |
| I-1-117 | 8-F | cyclo-C₃H₅ | 1 |
| I-1-118 | 8-F | CH₂-cyclo-C₃H₅ | 1 |
| I-1-119 | 8-F | C(CH₃)₃ | 1 |
| I-1-120 | 8-F | CH₂CF₃ | 1 |
| I-1-121 | 6-Cl | CH₃ | 1 |
| I-1-122 | 6-Cl | CH₃ | 2 |
| I-1-123 | 6-Cl | CH₂CH₃ | 1 |
| I-1-124 | 6-Cl | CH₂CH₃ | 2 |
| I-1-125 | 6-Cl | (CH₂)₂CH₃ | 1 |
| I-1-126 | 6-Cl | CH(CH₃)₂ | 1 |
| I-1-127 | 6-Cl | CH₂CHCH₂ | 1 |
| I-1-128 | 6-Cl | CH₂CCH | 1 |
| I-1-129 | 6-Cl | cyclo-C₃H₅ | 1 |
| I-1-130 | 6-Cl | CH₂-cyclo-C₃H₅ | 1 |
| I-1-131 | 6-Cl | C(CH₃)₃ | 1 |
| I-1-132 | 6-Cl | CH₂CF₃ | 1 |
| I-1-133 | 7-Cl | CH₃ | 1 |
| I-1-134 | 7-Cl | CH₃ | 2 |
| I-1-135 | 7-Cl | CH₂CH₃ | 1 |
| I-1-136 | 7-Cl | CH₂CH₃ | 2 |
| I-1-137 | 7-Cl | (CH₂)₂CH₃ | 1 |
| I-1-138 | 7-Cl | CH(CH₃)₂ | 1 |
| I-1-139 | 7-Cl | CH₂CHCH₂ | 1 |
| I-1-140 | 7-Cl | CH₂CCH | 1 |
| I-1-141 | 7-Cl | cyclo-C₃H₅ | 1 |
| I-1-142 | 7-Cl | CH₂-cyclo-C₃H₅ | 1 |
| I-1-143 | 7-Cl | C(CH₃)₃ | 1 |
| I-1-144 | 7-Cl | CH₂CF₃ | 1 |
| I-1-145 | 8-Cl | CH₃ | 1 |
| I-1-146 | 8-Cl | CH₃ | 2 |
| I-1-147 | 8-Cl | CH₂CH₃ | 1 |
| I-1-148 | 8-Cl | CH₂CH₃ | 2 |
| I-1-149 | 8-Cl | (CH₂)₂CH₃ | 1 |
| I-1-150 | 8-Cl | CH(CH₃)₂ | 1 |
| I-1-151 | 8-Cl | CH₂CHCH₂ | 1 |
| I-1-152 | 8-Cl | CH₂CCH | 1 |
| I-1-153 | 8-Cl | cyclo-C₃H₅ | 1 |
| I-1-154 | 8-Cl | CH₂-cyclo-C₃H₅ | 1 |
| I-1-155 | 8-Cl | C(CH₃)₃ | 1 |
| I-1-156 | 8-Cl | CH₂CF₃ | 1 |
| I-1-157 | 6-Br | CH₃ | 1 |
| I-1-158 | 6-Br | CH₃ | 2 |
| I-1-159 | 6-Br | CH₂CH₃ | 1 |
| I-1-160 | 6-Br | CH₂CH₃ | 2 |
| I-1-161 | 6-Br | (CH₂)₂CH₃ | 1 |
| I-1-162 | 6-Br | CH(CH₃)₂ | 1 |
| I-1-163 | 6-Br | CH₂CHCH₂ | 1 |
| I-1-164 | 6-Br | CH₂CCH | 1 |
| I-1-165 | 6-Br | cyclo-C₃H₅ | 1 |
| I-1-166 | 6-Br | CH₂-cyclo-C₃H₅ | 1 |
| I-1-167 | 6-Br | C(CH₃)₃ | 1 |
| I-1-168 | 6-Br | CH₂CF₃ | 1 |
| I-1-169 | 7-Br | CH₃ | 1 |
| I-1-170 | 7-Br | CH₃ | 2 |
| I-1-171 | 7-Br | CH₂CH₃ | 1 |
| I-1-172 | 7-Br | CH₂CH₃ | 2 |
| I-1-173 | 7-Br | (CH₂)₂CH₃ | 1 |
| I-1-174 | 7-Br | CH(CH₃)₂ | 1 |
| I-1-175 | 7-Br | CH₂CHCH₂ | 1 |
| I-1-176 | 7-Br | CH₂CCH | 1 |
| I-1-177 | 7-Br | cyclo-C₃H₅ | 1 |
| I-1-178 | 7-Br | CH₂-cyclo-C₃H₅ | 1 |
| I-1-179 | 7-Br | C(CH₃)₃ | 1 |
| I-1-180 | 7-Br | CH₂CF₃ | 1 |
| I-1-181 | 8-Br | CH₃ | 1 |
| I-1-182 | 8-Br | CH₃ | 2 |
| I-1-183 | 8-Br | CH₂CH₃ | 1 |
| I-1-184 | 8-Br | CH₂CH₃ | 2 |
| I-1-185 | 8-Br | (CH₂)₂CH₃ | 1 |
| I-1-186 | 8-Br | CH(CH₃)₂ | 1 |
| I-1-187 | 8-Br | CH₂CHCH₂ | 1 |
| I-1-188 | 8-Br | CH₂CCH | 1 |
| I-1-189 | 8-Br | cyclo-C₃H₅ | 1 |
| I-1-190 | 8-Br | CH₂-cyclo-C₃H₅ | 1 |
| I-1-191 | 8-Br | C(CH₃)₃ | 1 |
| I-1-192 | 8-Br | CH₂CF₃ | 1 |
| I-1-193 | 8-I | CH₃ | 1 |
| I-1-194 | 8-I | CH₃ | 2 |
| I-1-195 | 8-I | CH₂CH₃ | 1 |
| I-1-196 | 8-I | CH₂CH₃ | 2 |
| I-1-197 | 8-I | (CH₂)₂CH₃ | 1 |
| I-1-198 | 8-I | CH(CH₃)₂ | 1 |
| I-1-199 | 8-I | CH₂CHCH₂ | 1 |
| I-1-200 | 8-I | CH₂CCH | 1 |
| I-1-201 | 8-I | cyclo-C₃H₅ | 1 |
| I-1-202 | 8-I | CH₂-cyclo-C₃H₅ | 1 |
| I-1-203 | 8-I | C(CH₃)₃ | 1 |
| I-1-204 | 8-I | CH₂CF₃ | 1 |
| I-1-205 | 6-CH₃ | CH₃ | 1 |
| I-1-206 | 6-CH₃ | CH₃ | 2 |
| I-1-207 | 6-CH₃ | CH₂CH₃ | 1 |
| I-1-208 | 6-CH₃ | CH₂CH₃ | 2 |
| I-1-209 | 6-CH₃ | (CH₂)₂CH₃ | 1 |
| I-1-210 | 6-CH₃ | CH(CH₃)₂ | 1 |
| I-1-211 | 6-CH₃ | CH₂CHCH₂ | 1 |
| I-1-212 | 6-CH₃ | CH₂CCH | 1 |
| I-1-213 | 6-CH₃ | cyclo-C₃H₅ | 1 |
| I-1-214 | 6-CH₃ | CH₂-cyclo-C₃H₅ | 1 |
| I-1-215 | 6-CH₃ | C(CH₃)₃ | 1 |
| I-1-216 | 6-CH₃ | CH₂CF₃ | 1 |
| I-1-217 | 7-CH₃ | CH₃ | 1 |
| I-1-218 | 7-CH₃ | CH₃ | 2 |
| I-1-219 | 7-CH₃ | CH₂CH₃ | 1 |
| I-1-220 | 7-CH₃ | CH₂CH₃ | 2 |
| I-1-221 | 7-CH₃ | (CH₂)₂CH₃ | 1 |
| I-1-222 | 7-CH₃ | CH(CH₃)₂ | 1 |
| I-1-223 | 7-CH₃ | CH₂CHCH₂ | 1 |
| I-1-224 | 7-CH₃ | CH₂CCH | 1 |
| I-1-225 | 7-CH₃ | cyclo-C₃H₅ | 1 |
| I-1-226 | 7-CH₃ | CH₂-cyclo-C₃H₅ | 1 |
| I-1-227 | 7-CH₃ | C(CH₃)₃ | 1 |
| I-1-228 | 7-CH₃ | CH₂CF₃ | 1 |
| I-1-229 | 8-CH₃ | CH₃ | 1 |
| I-1-230 | 8-CH₃ | CH₃ | 2 |
| I-1-231 | 8-CH₃ | CH₂CH₃ | 1 |
| I-1-232 | 8-CH₃ | CH₂CH₃ | 2 |
| I-1-233 | 8-CH₃ | (CH₂)₂CH₃ | 1 |
| I-1-234 | 8-CH₃ | CH(CH₃)₂ | 1 |
| I-1-235 | 8-CH₃ | CH₂CHCH₂ | 1 |
| I-1-236 | 8-CH₃ | CH₂CCH | 1 |
| I-1-237 | 8-CH₃ | cyclo-C₃H₅ | 1 |
| I-1-238 | 8-CH₃ | CH₂-cyclo-C₃H₅ | 1 |
| I-1-239 | 8-CH₃ | C(CH₃)₃ | 1 |
| I-1-240 | 8-CH₃ | CH₂CF₃ | 1 |
| I-1-241 | 8-CH₂CH₃ | CH₃ | 1 |
| I-1-242 | 8-CH₂CH₃ | CH₃ | 2 |
| I-1-243 | 8-CH₂CH₃ | CH₂CH₃ | 1 |
| I-1-244 | 8-CH₂CH₃ | CH₂CH₃ | 2 |
| I-1-245 | 8-CH₂CH₃ | (CH₂)₂CH₃ | 1 |
| I-1-246 | 8-CH₂CH₃ | CH(CH₃)₂ | 1 |
| I-1-247 | 8-CH₂CH₃ | CH₂CHCH₂ | 1 |
| I-1-248 | 8-CH₂CH₃ | CH₂CCH | 1 |
| I-1-249 | 8-CH₂CH₃ | cyclo-C₃H₅ | 1 |
| I-1-250 | 8-CH₂CH₃ | CH₂-cyclo-C₃H₅ | 1 |
| I-1-251 | 8-CH₂CH₃ | C(CH₃)₃ | 1 |
| I-1-252 | 8-CH₂CH₃ | CH₂CF₃ | 1 |
| I-1-253 | 8-(CH₂)₂CH₃ | CH₃ | 1 |
| I-1-254 | 8-(CH₂)₂CH₃ | CH₃ | 2 |
| I-1-255 | 8-(CH₂)₂CH₃ | CH₂CH₃ | 1 |
| I-1-256 | 8-(CH₂)₂CH₃ | CH₂CH₃ | 2 |
| I-1-257 | 8-(CH₂)₂CH₃ | (CH₂)₂CH₃ | 1 |
| I-1-258 | 8-(CH₂)₂CH₃ | CH(CH₃)₂ | 1 |
| I-1-259 | 8-(CH₂)₂CH₃ | CH₂CHCH₂ | 1 |
| I-1-260 | 8-(CH₂)₂CH₃ | CH₂CCH | 1 |
| I-1-261 | 8-(CH₂)₂CH₃ | cyclo-C₃H₅ | 1 |
| I-1-262 | 8-(CH₂)₂CH₃ | CH₂-cyclo-C₃H₅ | 1 |
| I-1-263 | 8-(CH₂)₂CH₃ | C(CH₃)₃ | 1 |
| I-1-264 | 8-(CH₂)₂CH₃ | CH₂CF₃ | 1 |
| I-1-265 | 8-CH(CH₃)₂ | CH₃ | 1 |
| I-1-266 | 8-CH(CH₃)₂ | CH₃ | 2 |
| I-1-267 | 8-CH(CH₃)₂ | CH₂CH₃ | 1 |
| I-1-268 | 8-CH(CH₃)₂ | CH₂CH₃ | 2 |
| I-1-269 | 8-CH(CH₃)₂ | (CH₂)₂CH₃ | 1 |
| I-1-270 | 8-CH(CH₃)₂ | CH(CH₃)₂ | 1 |
| I-1-271 | 8-CH(CH₃)₂ | CH₂CHCH₂ | 1 |
| I-1-272 | 8-CH(CH₃)₂ | CH₂CCH | 1 |
| I-1-273 | 8-CH(CH₃)₂ | cyclo-C₃H₅ | 1 |
| I-1-274 | 8-CH(CH₃)₂ | CH₂-cyclo-C₃H₅ | 1 |
| I-1-275 | 8-CH(CH₃)₂ | C(CH₃)₃ | 1 |
| I-1-276 | 8-CH(CH₃)₂ | CH₂CF₃ | 1 |
| I-1-277 | 8-(CH₂)₃CH₃ | CH₃ | 1 |
| I-1-278 | 8-(CH₂)₃CH₃ | CH₃ | 2 |
| I-1-279 | 8-(CH₂)₃CH₃ | CH₂CH₃ | 1 |
| I-1-280 | 8-(CH₂)₃CH₃ | CH₂CH₃ | 2 |
| I-1-281 | 8-(CH₂)₃CH₃ | (CH₂)₂CH₃ | 1 |
| I-1-282 | 8-(CH₂)₃CH₃ | CH(CH₃)₂ | 1 |
| I-1-283 | 8-(CH₂)₃CH₃ | CH₂CHCH₂ | 1 |
| I-1-284 | 8-(CH₂)₃CH₃ | CH₂CCH | 1 |
| I-1-285 | 8-(CH₂)₃CH₃ | cyclo-C₃H₅ | 1 |
| I-1-286 | 8-(CH₂)₃CH₃ | CH₂-cyclo-C₃H₅ | 1 |
| I-1-287 | 8-(CH₂)₃CH₃ | C(CH₃)₃ | 1 |
| I-1-288 | 8-(CH₂)₃CH₃ | CH₂CF₃ | 1 |
| I-1-289 | 8-C(CH₃)₃ | CH₃ | 1 |
| I-1-290 | 8-C(CH₃)₃ | CH₃ | 2 |
| I-1-291 | 8-C(CH₃)₃ | CH₂CH₃ | 1 |
| I-1-292 | 8-C(CH₃)₃ | CH₂CH₃ | 2 |
| I-1-293 | 8-C(CH₃)₃ | (CH₂)₂CH₃ | 1 |
| I-1-294 | 8-C(CH₃)₃ | CH(CH₃)₂ | 1 |
| I-1-295 | 8-C(CH₃)₃ | CH₂CHCH₂ | 1 |
| I-1-296 | 8-C(CH₃)₃ | CH₂CCH | 1 |
| I-1-297 | 8-C(CH₃)₃ | cyclo-C₃H₅ | 1 |
| I-1-298 | 8-C(CH₃)₃ | CH₂-cyclo-C₃H₅ | 1 |
| I-1-299 | 8-C(CH₃)₃ | C(CH₃)₃ | 1 |
| I-1-300 | 8-C(CH₃)₃ | CH₂CF₃ | 1 |
| I-1-301 | 8-CH₂CHCH₂ | CH₃ | 1 |
| I-1-302 | 8-CH₂CHCH₂ | CH₃ | 2 |
| I-1-303 | 8-CH₂CHCH₂ | CH₂CH₃ | 1 |
| I-1-304 | 8-CH₂CHCH₂ | CH₂CH₃ | 2 |
| I-1-305 | 8-CH₂CHCH₂ | (CH₂)₂CH₃ | 1 |
| I-1-306 | 8-CH₂CHCH₂ | CH(CH₃)₂ | 1 |
| I-1-307 | 8-CH₂CHCH₂ | CH₂CHCH₂ | 1 |
| I-1-308 | 8-CH₂CHCH₂ | CH₂CCH | 1 |
| I-1-309 | 8-CH₂CHCH₂ | cyclo-C₃H₅ | 1 |
| I-1-310 | 8-CH₂CHCH₂ | CH₂-cyclo-C₃H₅ | 1 |
| I-1-311 | 8-CH₂CHCH₂ | C(CH₃)₃ | 1 |
| I-1-312 | 8-CH₂CHCH₂ | CH₂CF₃ | 1 |
| I-1-313 | 8-CH₂CHCH | CH₃ | 1 |
| I-1-314 | 8-CH₂CHCH | CH₃ | 2 |
| I-1-315 | 8-CH₂CHCH | CH₂CH₃ | 1 |
| I-1-316 | 8-CH₂CHCH | CH₂CH₃ | 2 |
| I-1-317 | 8-CH₂CHCH | (CH₂)₂CH₃ | 1 |
| I-1-318 | 8-CH₂CHCH | CH(CH₃)₂ | 1 |
| I-1-319 | 8-CH₂CHCH | CH₂CHCH₂ | 1 |
| I-1-320 | 8-CH₂CHCH | CH₂CCH | 1 |
| I-1-321 | 8-CH₂CHCH | cyclo-C₃H₅ | 1 |
| I-1-322 | 8-CH₂CHCH | CH₂-cyclo-C₃H₅ | 1 |
| I-1-323 | 8-CH₂CHCH | C(CH₃)₃ | 1 |
| I-1-324 | 8-CH₂CHCH | CH₂CF₃ | 1 |
| I-1-325 | 6-CF₃ | CH₃ | 1 |
| I-1-326 | 6-CF₃ | CH₃ | 2 |
| I-1-327 | 6-CF₃ | CH₂CH₃ | 1 |
| I-1-328 | 6-CF₃ | CH₂CH₃ | 2 |
| I-1-329 | 6-CF₃ | (CH₂)₂CH₃ | 1 |
| I-1-330 | 6-CF₃ | CH(CH₃)₂ | 1 |
| I-1-331 | 6-CF₃ | CH₂CHCH₂ | 1 |
| I-1-332 | 6-CF₃ | CH₂CCH | 1 |
| I-1-333 | 6-CF₃ | cyclo-C₃H₅ | 1 |
| I-1-334 | 6-CF₃ | CH₂-cyclo-C₃H₅ | 1 |
| I-1-335 | 6-CF₃ | C(CH₃)₃ | 1 |
| I-1-336 | 6-CF₃ | CH₂CF₃ | 1 |
| I-1-337 | 7-CF₃ | CH₃ | 1 |
| I-1-338 | 7-CF₃ | CH₃ | 2 |
| I-1-339 | 7-CF₃ | CH₂CH₃ | 1 |
| I-1-340 | 7-CF₃ | CH₂CH₃ | 2 |
| I-1-341 | 7-CF₃ | (CH₂)₂CH₃ | 1 |
| I-1-342 | 7-CF₃ | CH(CH₃)₂ | 1 |
| I-1-343 | 7-CF₃ | CH₂CHCH₂ | 1 |
| I-1-344 | 7-CF₃ | CH₂CCH | 1 |
| I-1-345 | 7-CF₃ | cyclo-C₃H₅ | 1 |
| I-1-346 | 7-CF₃ | CH₂-cyclo-C₃H₅ | 1 |
| I-1-347 | 7-CF₃ | C(CH₃)₃ | 1 |
| I-1-348 | 7-CF₃ | CH₂CF₃ | 1 |
| I-1-349 | 8-CF₃ | CH₃ | 1 |
| I-1-350 | 8-CF₃ | CH₃ | 2 |
| I-1-351 | 8-CF₃ | CH₂CH₃ | 1 |
| I-1-352 | 8-CF₃ | CH₂CH₃ | 2 |
| I-1-353 | 8-CF₃ | (CH₂)₂CH₃ | 1 |
| I-1-354 | 8-CF₃ | CH(CH₃)₂ | 1 |
| I-1-355 | 8-CF₃ | CH₂CHCH₂ | 1 |
| I-1-356 | 8-CF₃ | CH₂CCH | 1 |
| I-1-357 | 8-CF₃ | cyclo-C₃H₅ | 1 |
| I-1-358 | 8-CF₃ | CH₂-cyclo-C₃H₅ | 1 |
| I-1-359 | 8-CF₃ | C(CH₃)₃ | 1 |
| I-1-360 | 8-CF₃ | CH₂CF₃ | 1 |
| I-1-361 | 6-OCH₃ | CH₃ | 1 |
| I-1-362 | 6-OCH₃ | CH₃ | 2 |
| I-1-363 | 6-OCH₃ | CH₂CH₃ | 1 |
| I-1-364 | 6-OCH₃ | CH₂CH₃ | 2 |
| I-1-365 | 6-OCH₃ | (CH₂)₂CH₃ | 1 |
| I-1-366 | 6-OCH₃ | CH(CH₃)₂ | 1 |
| I-1-367 | 6-OCH₃ | CH₂CHCH₂ | 1 |
| I-1-368 | 6-OCH₃ | CH₂CCH | 1 |
| I-1-369 | 6-OCH₃ | cyclo-C₃H₅ | 1 |
| I-1-370 | 6-OCH₃ | CH₂-cyclo-C₃H₅ | 1 |
| I-1-371 | 6-OCH₃ | C(CH₃)₃ | 1 |
| I-1-372 | 6-OCH₃ | CH₂CF₃ | 1 |
| I-1-373 | 7-OCH₃ | CH₃ | 1 |
| I-1-374 | 7-OCH₃ | CH₃ | 2 |
| I-1-375 | 7-OCH₃ | CH₂CH₃ | 1 |
| I-1-376 | 7-OCH₃ | CH₂CH₃ | 2 |
| I-1-377 | 7-OCH₃ | (CH₂)₂CH₃ | 1 |
| I-1-378 | 7-OCH₃ | CH(CH₃)₂ | 1 |
| I-1-379 | 7-OCH₃ | CH₂CHCH₂ | 1 |
| I-1-380 | 7-OCH₃ | CH₂CCH | 1 |
| I-1-381 | 7-OCH₃ | cyclo-C₃H₅ | 1 |
| I-1-382 | 7-OCH₃ | CH₂-cyclo-C₃H₅ | 1 |
| I-1-383 | 7-OCH₃ | C(CH₃)₃ | 1 |
| I-1-384 | 7-OCH₃ | CH₂CF₃ | 1 |
| I-1-385 | 8-OCH₃ | CH₃ | 1 |
| I-1-386 | 8-OCH₃ | CH₃ | 2 |
| I-1-387 | 8-OCH₃ | CH₂CH₃ | 1 |
| I-1-388 | 8-OCH₃ | CH₂CH₃ | 2 |
| I-1-389 | 8-OCH₃ | (CH₂)₂CH₃ | 1 |
| I-1-390 | 8-OCH₃ | CH(CH₃)₂ | 1 |
| I-1-391 | 8-OCH₃ | CH₂CHCH₂ | 1 |
| I-1-392 | 8-OCH₃ | CH₂CCH | 1 |
| I-1-393 | 8-OCH₃ | cyclo-C₃H₅ | 1 |
| I-1-394 | 8-OCH₃ | CH₂-cyclo-C₃H₅ | 1 |
| I-1-395 | 8-OCH₃ | C(CH₃)₃ | 1 |
| I-1-396 | 8-OCH₃ | CH₂CF₃ | 1 |
| I-1-397 | 6-OCF₃ | CH₃ | 1 |
| I-1-398 | 6-OCF₃ | CH₃ | 2 |
| I-1-399 | 6-OCF₃ | CH₂CH₃ | 1 |
| I-1-400 | 6-OCF₃ | CH₂CH₃ | 2 |
| I-1-401 | 6-OCF₃ | (CH₂)₂CH₃ | 1 |
| I-1-402 | 6-OCF₃ | CH(CH₃)₂ | 1 |
| I-1-403 | 6-OCF₃ | CH₂CHCH₂ | 1 |
| I-1-404 | 6-OCF₃ | CH₂CCH | 1 |
| I-1-405 | 6-OCF₃ | cyclo-C₃H₅ | 1 |
| I-1-406 | 6-OCF₃ | CH₂-cyclo-C₃H₅ | 1 |
| I-1-407 | 6-OCF₃ | C(CH₃)₃ | 1 |
| I-1-408 | 6-OCF₃ | CH₂CF₃ | 1 |
| I-1-409 | 7-OCF₃ | CH₃ | 1 |
| I-1-410 | 7-OCF₃ | CH₃ | 2 |
| I-1-411 | 7-OCF₃ | CH₂CH₃ | 1 |
| I-1-412 | 7-OCF₃ | CH₂CH₃ | 2 |
| I-1-413 | 7-OCF₃ | (CH₂)₂CH₃ | 1 |
| I-1-414 | 7-OCF₃ | CH(CH₃)₂ | 1 |
| I-1-415 | 7-OCF₃ | CH₂CHCH₂ | 1 |
| I-1-416 | 7-OCF₃ | CH₂CCH | 1 |
| I-1-417 | 7-OCF₃ | cyclo-C₃H₅ | 1 |
| I-1-418 | 7-OCF₃ | CH₂-cyclo-C₃H₅ | 1 |
| I-1-419 | 7-OCF₃ | C(CH₃)₃ | 1 |
| I-1-420 | 7-OCF₃ | CH₂CF₃ | 1 |
| I-1-421 | 8-OCF₃ | CH₃ | 1 |
| I-1-422 | 8-OCF₃ | CH₃ | 2 |
| I-1-423 | 8-OCF₃ | CH₂CH₃ | 1 |
| I-1-424 | 8-OCF₃ | CH₂CH₃ | 2 |
| I-1-425 | 8-OCF₃ | (CH₂)₂CH₃ | 1 |
| I-1-426 | 8-OCF₃ | CH(CH₃)₂ | 1 |
| I-1-427 | 8-OCF₃ | CH₂CHCH₂ | 1 |
| I-1-428 | 8-OCF₃ | CH₂CCH | 1 |
| I-1-429 | 8-OCF₃ | cyclo-C₃H₅ | 1 |
| I-1-430 | 8-OCF₃ | CH₂-cyclo-C₃H₅ | 1 |
| I-1-431 | 8-OCF₃ | C(CH₃)₃ | 1 |
| I-1-432 | 8-OCF₃ | CH₂CF₃ | 1 |
| I-1-433 | 8-SCH₃ | CH₃ | 1 |
| I-1-434 | 8-SCH₃ | CH₃ | 2 |
| I-1-435 | 8-SCH₃ | CH₂CH₃ | 1 |
| I-1-436 | 8-SCH₃ | CH₂CH₃ | 2 |
| I-1-437 | 8-SCH₃ | (CH₂)₂CH₃ | 1 |
| I-1-438 | 8-SCH₃ | CH(CH₃)₂ | 1 |
| I-1-439 | 8-SCH₃ | CH₂CHCH₂ | 1 |
| I-1-440 | 8-SCH₃ | CH₂CCH | 1 |
| I-1-441 | 8-SCH₃ | cyclo-C₃H₅ | 1 |
| I-1-442 | 8-SCH₃ | CH₂-cyclo-C₃H₅ | 1 |
| I-1-443 | 8-SCH₃ | C(CH₃)₃ | 1 |
| I-1-444 | 8-SCH₃ | CH₂CF₃ | 1 |
| I-1-445 | 8-SCH₂CH₃ | CH₃ | 1 |
| I-1-446 | 8-SCH₂CH₃ | CH₃ | 2 |
| I-1-447 | 8-SCH₂CH₃ | CH₂CH₃ | 1 |
| I-1-448 | 8-SCH₂CH₃ | CH₂CH₃ | 2 |
| I-1-449 | 8-SCH₂CH₃ | (CH₂)₂CH₃ | 1 |
| I-1-450 | 8-SCH₂CH₃ | CH(CH₃)₂ | 1 |
| I-1-451 | 8-SCH₂CH₃ | CH₂CHCH₂ | 1 |
| I-1-452 | 8-SCH₂CH₃ | CH₂CCH | 1 |
| I-1-453 | 8-SCH₂CH₃ | cyclo-C₃H₅ | 1 |
| I-1-454 | 8-SCH₂CH₃ | CH₂-cyclo-C₃H₅ | 1 |
| I-1-455 | 8-SCH₂CH₃ | C(CH₃)₃ | 1 |
| I-1-456 | 8-SCH₂CH₃ | CH₂CF₃ | 1 |
| I-1-457 | 8-SCH(CH₃)₂ | CH₃ | 1 |
| I-1-458 | 8-SCH(CH₃)₂ | CH₃ | 2 |
| I-1-459 | 8-SCH(CH₃)₂ | CH₂CH₃ | 1 |
| I-1-460 | 8-SCH(CH₃)₂ | CH₂CH₃ | 2 |
| I-1-461 | 8-SCH(CH₃)₂ | (CH₂)₂CH₃ | 1 |
| I-1-462 | 8-SCH(CH₃)₂ | CH(CH₃)₂ | 1 |
| I-1-463 | 8-SCH(CH₃)₂ | CH₂CHCH₂ | 1 |
| I-1-464 | 8-SCH(CH₃)₂ | CH₂CCH | 1 |
| I-1-465 | 8-SCH(CH₃)₂ | cyclo-C₃H₅ | 1 |
| I-1-466 | 8-SCH(CH₃)₂ | CH₂-cyclo-C₃H₅ | 1 |
| I-1-467 | 8-SCH(CH₃)₂ | C(CH₃)₃ | 1 |
| I-1-468 | 8-SCH(CH₃)₂ | CH₂CF₃ | 1 |
| I-1-469 | 8-SCH₂CCH | CH₃ | 1 |
| I-1-470 | 8-SCH₂CCH | CH₃ | 2 |
| I-1-471 | 8-SCH₂CCH | CH₂CH₃ | 1 |
| I-1-472 | 8-SCH₂CCH | CH₂CH₃ | 2 |
| I-1-473 | 8-SCH₂CCH | (CH₂)₂CH₃ | 1 |
| I-1-474 | 8-SCH₂CCH | CH(CH₃)₂ | 1 |
| I-1-475 | 8-SCH₂CCH | CH₂CHCH₂ | 1 |
| I-1-476 | 8-SCH₂CCH | CH₂CCH | 1 |
| I-1-477 | 8-SCH₂CCH | cyclo-C₃H₅ | 1 |
| I-1-478 | 8-SCH₂CCH | CH₂-cyclo-C₃H₅ | 1 |
| I-1-479 | 8-SCH₂CCH | C(CH₃)₃ | 1 |
| I-1-480 | 8-SCH₂CCH | CH₂CF₃ | 1 |
| I-1-481 | 6-SCF₃ | CH₃ | 1 |
| I-1-482 | 6-SCF₃ | CH₃ | 2 |
| I-1-483 | 6-SCF₃ | CH₂CH₃ | 1 |
| I-1-484 | 6-SCF₃ | CH₂CH₃ | 2 |
| I-1-485 | 6-SCF₃ | (CH₂)₂CH₃ | 1 |
| I-1-486 | 6-SCF₃ | CH(CH₃)₂ | 1 |
| I-1-487 | 6-SCF₃ | CH₂CHCH₂ | 1 |
| I-1-488 | 6-SCF₃ | CH₂CCH | 1 |
| I-1-489 | 6-SCF₃ | cyclo-C₃H₅ | 1 |
| I-1-490 | 6-SCF₃ | CH₂-cyclo-C₃H₅ | 1 |
| I-1-491 | 6-SCF₃ | C(CH₃)₃ | 1 |
| I-1-492 | 6-SCF₃ | CH₂CF₃ | 1 |
| I-1-493 | 7-SCF₃ | CH₃ | 1 |
| I-1-494 | 7-SCF₃ | CH₃ | 2 |
| I-1-495 | 7-SCF₃ | CH₂CH₃ | 1 |
| I-1-496 | 7-SCF₃ | CH₂CH₃ | 2 |
| I-1-497 | 7-SCF₃ | (CH₂)₂CH₃ | 1 |
| I-1-498 | 7-SCF₃ | CH(CH₃)₂ | 1 |
| I-1-499 | 7-SCF₃ | CH₂CHCH₂ | 1 |
| I-1-500 | 7-SCF₃ | CH₂CCH | 1 |
| I-1-501 | 7-SCF₃ | cyclo-C₃H₅ | 1 |
| I-1-502 | 7-SCF₃ | CH₂-cyclo-C₃H₅ | 1 |
| I-1-503 | 7-SCF₃ | C(CH₃)₃ | 1 |
| I-1-504 | 7-SCF₃ | CH₂CF₃ | 1 |
| I-1-505 | 8-SCF₃ | CH₃ | 1 |
| I-1-506 | 8-SCF₃ | CH₃ | 2 |
| I-1-507 | 8-SCF₃ | CH₂CH₃ | 1 |
| I-1-508 | 8-SCF₃ | CH₂CH₃ | 2 |
| I-1-509 | 8-SCF₃ | (CH₂)₂CH₃ | 1 |
| I-1-510 | 8-SCF₃ | CH(CH₃)₂ | 1 |
| I-1-511 | 8-SCF₃ | CH₂CHCH₂ | 1 |
| I-1-512 | 8-SCF₃ | CH₂CCH | 1 |
| I-1-513 | 8-SCF₃ | cyclo-C₃H₅ | 1 |
| I-1-514 | 8-SCF₃ | CH₂-cyclo-C₃H₅ | 1 |
| I-1-515 | 8-SCF₃ | C(CH₃)₃ | 1 |
| I-1-516 | 8-SCF₃ | CH₂CF₃ | 1 |
| I-1-517 | 8-cyclo-C₃H₅ | CH₃ | 1 |
| I-1-518 | 8-cyclo-C₃H₅ | CH₃ | 2 |
| I-1-519 | 8-cyclo-C₃H₅ | CH₂CH₃ | 1 |
| I-1-520 | 8-cyclo-C₃H₅ | CH₂CH₃ | 2 |
| I-1-521 | 8-cyclo-C₃H₅ | (CH₂)₂CH₃ | 1 |
| I-1-522 | 8-cyclo-C₃H₅ | CH(CH₃)₂ | 1 |
| I-1-523 | 8-cyclo-C₃H₅ | CH₂CHCH₂ | 1 |
| I-1-524 | 8-cyclo-C₃H₅ | CH₂CCH | 1 |
| I-1-525 | 8-cyclo-C₃H₅ | cyclo-C₃H₅ | 1 |
| I-1-526 | 8-cyclo-C₃H₅ | CH₂-cyclo-C₃H₅ | 1 |
| I-1-527 | 8-cyclo-C₃H₅ | C(CH₃)₃ | 1 |
| I-1-528 | 8-cyclo-C₃H₅ | CH₂CF₃ | 1 |
| I-1-529 | 8-CH₂-cyclo-C₃H₅ | CH₃ | 1 |
| I-1-530 | 8-CH₂-cyclo-C₃H₅ | CH₃ | 2 |
| I-1-531 | 8-CH₂-cyclo-C₃H₅ | CH₂CH₃ | 1 |
| I-1-532 | 8-CH₂-cyclo-C₃H₅ | CH₂CH₃ | 2 |
| I-1-533 | 8-CH₂-cyclo-C₃H₅ | (CH₂)₂CH₃ | 1 |
| I-1-534 | 8-CH₂-cyclo-C₃H₅ | CH(CH₃)₂ | 1 |
| I-1-535 | 8-CH₂-cyclo-C₃H₅ | CH₂CHCH₂ | 1 |
| I-1-536 | 8-CH₂-cyclo-C₃H₅ | CH₂CCH | 1 |
| I-1-537 | 8-CH₂-cyclo-C₃H₅ | cyclo-C₃H₅ | 1 |
| I-1-538 | 8-CH₂-cyclo-C₃H₅ | CH₂-cyclo-C₃H₅ | 1 |
| I-1-539 | 8-CH₂-cyclo-C₃H₅ | C(CH₃)₃ | 1 |
| I-1-540 | 8-CH₂-cyclo-C₃H₅ | CH₂CF₃ | 1 |
| I-1-541 | 6-F,8-CH₃ | CH₃ | 1 |
| I-1-542 | 6-F,8-CH₃ | CH₂CH₃ | 1 |
| I-1-543 | 6-F,8-CH₃ | CH₂CHCH₂ | 1 |
| I-1-544 | 6-F,8-CH₃ | CH₂CCH | 1 |
| I-1-545 | 6-F,8-CH₃ | cyclo-C₃H₅ | 1 |
| I-1-546 | 6-F,8-CH₃ | CH₂-cyclo-C₃H₅ | 1 |
| I-1-547 | 6-F,8-CH₃ | CH₂CF₃ | 1 |
| I-1-548 | 6-F,8-CH₂CH₃ | CH₃ | 1 |
| I-1-549 | 6-F,8-CH₂CH₃ | CH₂CH₃ | 1 |
| I-1-550 | 6-F,8-CH₂CH₃ | CH₂CHCH₂ | 1 |
| I-1-551 | 6-F,8-CH₂CH₃ | CH₂CCH | 1 |
| I-1-552 | 6-F,8-CH₂CH₃ | cyclo-C₃H₅ | 1 |
| I-1-553 | 6-F,8-CH₂CH₃ | CH₂-cyclo-C₃H₅ | 1 |
| I-1-554 | 6-F,8-CH₂CH₃ | CH₂CF₃ | 1 |
| I-1-555 | 6-F,8-(CH₂)₂CH₃ | CH₃ | 1 |
| I-1-556 | 6-F,8-(CH₂)₂CH₃ | CH₂CH₃ | 1 |
| I-1-557 | 6-F,8-(CH₂)₂CH₃ | CH₂CHCH₂ | 1 |
| I-1-558 | 6-F,8-(CH₂)₂CH₃ | CH₂CCH | 1 |
| I-1-559 | 6-F,8-(CH₂)₂CH₃ | cyclo-C₃H₅ | 1 |
| I-1-560 | 6-F,8-(CH₂)₂CH₃ | CH₂-cyclo-C₃H₅ | 1 |
| I-1-561 | 6-F,8-(CH₂)₂CH₃ | CH₂CF₃ | 1 |
| I-1-562 | 6-F,8-CH(CH₃)₂ | CH₃ | 1 |
| I-1-563 | 6-F,8-CH(CH₃)₂ | CH₂CH₃ | 1 |
| I-1-564 | 6-F,8-CH(CH₃)₂ | CH₂CHCH₂ | 1 |
| I-1-565 | 6-F,8-CH(CH₃)₂ | CH₂CCH | 1 |
| I-1-566 | 6-F,8-CH(CH₃)₂ | cyclo-C₃H₅ | 1 |
| I-1-567 | 6-F,8-CH(CH₃)₂ | CH₂-cyclo-C₃H₅ | 1 |
| I-1-568 | 6-F,8-CH(CH₃)₂ | CH₂CF₃ | 1 |
| I-1-569 | 6-F,8-(CH₂)₃CH₃ | CH₃ | 1 |
| I-1-570 | 6-F,8-(CH₂)₃CH₃ | CH₂CH₃ | 1 |
| I-1-571 | 6-F,8-(CH₂)₃CH₃ | CH₂CHCH₂ | 1 |
| I-1-572 | 6-F,8-(CH₂)₃CH₃ | CH₂CCH | 1 |
| I-1-573 | 6-F,8-(CH₂)₃CH₃ | cyclo-C₃H₅ | 1 |
| I-1-574 | 6-F,8-(CH₂)₃CH₃ | CH₂-cyclo-C₃H₅ | 1 |
| I-1-575 | 6-F,8-(CH₂)₃CH₃ | CH₂CF₃ | 1 |
| I-1-576 | 6-F,8-C(CH₃)₃ | CH₃ | 1 |
| I-1-577 | 6-F,8-C(CH₃)₃ | CH₂CH₃ | 1 |
| I-1-578 | 6-F,8-C(CH₃)₃ | CH₂CHCH₂ | 1 |
| I-1-579 | 6-F,8-C(CH₃)₃ | CH₂CCH | 1 |
| I-1-580 | 6-F,8-C(CH₃)₃ | cyclo-C₃H₅ | 1 |
| I-1-581 | 6-F,8-C(CH₃)₃ | CH₂-cyclo-C₃H₅ | 1 |
| I-1-582 | 6-F,8-C(CH₃)₃ | CH₂CF₃ | 1 |
| I-1-583 | 6-F,8-CH₂CHCH₂ | CH₃ | 1 |
| I-1-584 | 6-F,8-CH₂CHCH₂ | CH₂CH₃ | 1 |
| I-1-585 | 6-F,8-CH₂CHCH₂ | CH₂CHCH₂ | 1 |
| I-1-586 | 6-F,8-CH₂CHCH₂ | CH₂CCH | 1 |
| I-1-587 | 6-F,8-CH₂CHCH₂ | cyclo-C₃H₅ | 1 |
| I-1-588 | 6-F,8-CH₂CHCH₂ | CH₂-cyclo-C₃H₅ | 1 |
| I-1-589 | 6-F,8-CH₂CHCH₂ | CH₂CF₃ | 1 |
| I-1-590 | 6-F,8-CH₂CCH | CH₃ | 1 |
| I-1-591 | 6-F,8-CH₂CCH | CH₂CH₃ | 1 |
| I-1-592 | 6-F,8-CH₂CCH | CH₂CHCH₂ | 1 |
| I-1-593 | 6-F,8-CH₂CCH | CH₂CCH | 1 |
| I-1-594 | 6-F,8-CH₂CCH | cyclo-C₃H₅ | 1 |
| I-1-595 | 6-F,8-CH₂CCH | CH₂-cyclo-C₃H₅ | 1 |
| I-1-596 | 6-F,8-CH₂CCH | CH₂CF₃ | 1 |
| I-1-597 | 6-F,8-cyclo-C₃H₅ | CH₃ | 1 |
| I-1-598 | 6-F,8-cyclo-C₃H₅ | CH₂CH₃ | 1 |
| I-1-599 | 6-F,8-cyclo-C₃H₅ | CH₂CHCH₂ | 1 |
| I-1-600 | 6-F,8-cyclo-C₃H₅ | CH₂CCH | 1 |
| I-1-601 | 6-F,8-cyclo-C₃H₅ | cyclo-C₃H₅ | 1 |
| I-1-602 | 6-F,8-cyclo-C₃H₅ | CH₂-cyclo-C₃H₅ | 1 |
| I-1-603 | 6-F,8-cyclo-C₃H₅ | CH₂CF₃ | 1 |
| I-1-604 | 6-F,8-CH₂-cyclo-C₃H₅ | CH₃ | 1 |
| I-1-605 | 6-F,8-CH₂-cyclo-C₃H₅ | CH₂CH₃ | 1 |
| I-1-606 | 6-F,8-CH₂-cyclo-C₃H₅ | CH₂CHCH₂ | 1 |
| I-1-607 | 6-F,8-CH₂-cyclo-C₃H₅ | CH₂CCH | 1 |
| I-1-608 | 6-F,8-CH₂-cyclo-C₃H₅ | cyclo-C₃H₅ | 1 |
| I-1-609 | 6-F,8-CH₂-cyclo-C₃H₅ | CH₂-cyclo-C₃H₅ | 1 |
| I-1-610 | 6-F,8-CH₂-cyclo-C₃H₅ | CH₂CF₃ | 1 |
| I-1-611 | 6-F,8-SCH₃ | CH₃ | 1 |
| I-1-612 | 6-F,8-SCH₃ | CH₂CH₃ | 1 |
| I-1-613 | 6-F,8-SCH₃ | CH₂CHCH₂ | 1 |
| I-1-614 | 6-F,8-SCH₃ | CH₂CCH | 1 |
| I-1-615 | 6-F,8-SCH₃ | cyclo-C₃H₅ | 1 |
| I-1-616 | 6-F,8-SCH₃ | CH₂-cyclo-C₃H₅ | 1 |
| I-1-617 | 6-F,8-SCH₃ | CH₂CF₃ | 1 |
| I-1-618 | 6-F,8-SCH₂CH₃ | CH₃ | 1 |
| I-1-619 | 6-F,8-SCH₂CH₃ | CH₂CH₃ | 1 |
| I-1-620 | 6-F,8-SCH₂CH₃ | CH₂CHCH₂ | 1 |
| I-1-621 | 6-F,8-SCH₂CH₃ | CH₂CCH | 1 |
| I-1-622 | 6-F,8-SCH₂CH₃ | cyclo-C₃H₅ | 1 |
| I-1-623 | 6-F,8-SCH₂CH₃ | CH₂-cyclo-C₃H₅ | 1 |
| I-1-624 | 6-F,8-SCH₂CH₃ | CH₂CF₃ | 1 |
| I-1-625 | 6-F,8-SCH₂CCH | CH₃ | 1 |
| I-1-626 | 6-F,8-SCH₂CCH | CH₂CH₃ | 1 |
| I-1-627 | 6-F,8-SCH₂CCH | CH₂CHCH₂ | 1 |
| I-1-628 | 6-F,8-SCH₂CCH | CH₂CCH | 1 |
| I-1-629 | 6-F,8-SCH₂CCH | cyclo-C₃H₅ | 1 |
| I-1-630 | 6-F,8-SCH₂CCH | CH₂-cyclo-C₃H₅ | 1 |
| I-1-631 | 6-F,8-SCH₂CCH | CH₂CF₃ | 1 |
| I-1-632 | 6-F,8-SCH(CH₃)₂ | CH₃ | 1 |
| I-1-633 | 6-F,8-SCH(CH₃)₂ | CH₂CH₃ | 1 |
| I-1-634 | 6-F,8-SCH(CH₃)₂ | CH₂CHCH₂ | 1 |
| I-1-635 | 6-F,8-SCH(CH₃)₂ | CH₂CCH | 1 |
| I-1-636 | 6-F,8-SCH(CH₃)₂ | cyclo-C₃H₅ | 1 |
| I-1-637 | 6-F,8-SCH(CH₃)₂ | CH₂-cyclo-C₃H₅ | 1 |
| I-1-638 | 6-F,8-SCH(CH₃)₂ | CH₂CF₃ | 1 |
| I-1-639 | 6-Cl,8-CH₃ | CH₃ | 1 |
| I-1-640 | 6-Cl,8-CH₃ | CH₂CH₃ | 1 |
| I-1-641 | 6-Cl,8-CH₃ | CH₂CHCH₂ | 1 |
| I-1-642 | 6-Cl,8-CH₃ | CH₂CCH | 1 |
| I-1-643 | 6-Cl,8-CH₃ | cyclo-C₃H₅ | 1 |
| I-1-644 | 6-Cl,8-CH₃ | CH₂-cyclo-C₃H₅ | 1 |
| I-1-645 | 6-Cl,8-CH₃ | CH₂CF₃ | 1 |
| I-1-646 | 6-Cl,8-CH₂CH₃ | CH₃ | 1 |
| I-1-647 | 6-Cl,8-CH₂CH₃ | CH₂CH₃ | 1 |
| I-1-648 | 6-Cl,8-CH₂CH₃ | CH₂CHCH₂ | 1 |
| I-1-649 | 6-Cl,8-CH₂CH₃ | CH₂CCH | 1 |
| I-1-650 | 6-Cl,8-CH₂CH₃ | cyclo-C₃H₅ | 1 |
| I-1-651 | 6-Cl,8-CH₂CH₃ | CH₂-cyclo-C₃H₅ | 1 |
| I-1-652 | 6-Cl,8-CH₂CH₃ | CH₂CF₃ | 1 |
| I-1-653 | 6-Cl,8-(CH₂)₂CH₃ | CH₃ | 1 |
| I-1-654 | 6-Cl,8-(CH₂)₂CH₃ | CH₂CH₃ | 1 |
| I-1-655 | 6-Cl,8-(CH₂)₂CH₃ | CH₂CHCH₂ | 1 |
| I-1-656 | 6-Cl,8-(CH₂)₂CH₃ | CH₂CCH | 1 |
| I-1-657 | 6-Cl,8-(CH₂)₂CH₃ | cyclo-C₃H₅ | 1 |
| I-1-658 | 6-Cl,8-(CH₂)₂CH₃ | CH₂-cyclo-C₃H₅ | 1 |
| I-1-659 | 6-Cl,8-(CH₂)₂CH₃ | CH₂CF₃ | 1 |
| I-1-660 | 6-Cl,8-CH(CH₃)₂ | CH₃ | 1 |
| I-1-661 | 6-Cl,8-CH(CH₃)₂ | CH₂CH₃ | 1 |
| I-1-662 | 6-Cl,8-CH(CH₃)₂ | CH₂CHCH₂ | 1 |
| I-1-663 | 6-Cl,8-CH(CH₃)₂ | CH₂CCH | 1 |
| I-1-664 | 6-Cl,8-CH(CH₃)₂ | cyclo-C₃H₅ | 1 |
| I-1-665 | 6-Cl,8-CH(CH₃)₂ | CH₂-cyclo-C₃H₅ | 1 |
| I-1-666 | 6-Cl,8-CH(CH₃)₂ | CH₂CF₃ | 1 |
| I-1-667 | 6-Cl,8-(CH₂)₃CH₃ | CH₃ | 1 |
| I-1-668 | 6-Cl,8-(CH₂)₃CH₃ | CH₂CH₃ | 1 |
| I-1-669 | 6-Cl,8-(CH₂)₃CH₃ | CH₂CHCH₂ | 1 |
| I-1-670 | 6-Cl,8-(CH₂)₃CH₃ | CH₂CCH | 1 |
| I-1-671 | 6-Cl,8-(CH₂)₃CH₃ | cyclo-C₃H₅ | 1 |
| I-1-672 | 6-Cl,8-(CH₂)₃CH₃ | CH₂-cyclo-C₃H₅ | 1 |
| I-1-673 | 6-Cl,8-(CH₂)₃CH₃ | CH₂CF₃ | 1 |
| I-1-674 | 6-Cl,8-C(CH₃)₃ | CH₃ | 1 |
| I-1-675 | 6-Cl,8-C(CH₃)₃ | CH₂CH₃ | 1 |
| I-1-676 | 6-Cl,8-C(CH₃)₃ | CH₂CHCH₂ | 1 |
| I-1-677 | 6-Cl,8-C(CH₃)₃ | CH₂CCH | 1 |
| I-1-678 | 6-Cl,8-C(CH₃)₃ | cyclo-C₃H₅ | 1 |
| I-1-679 | 6-Cl,8-C(CH₃)₃ | CH₂-cyclo-C₃H₅ | 1 |
| I-1-680 | 6-Cl,8-C(CH₃)₃ | CH₂CF₃ | 1 |
| I-1-681 | 6-Cl,8-CH₂CHCH₂ | CH₃ | 1 |
| I-1-682 | 6-Cl,8-CH₂CHCH₂ | CH₂CH₃ | 1 |
| I-1-683 | 6-Cl,8-CH₂CHCH₂ | CH₂CHCH₂ | 1 |
| I-1-684 | 6-Cl,8-CH₂CHCH₂ | CH₂CCH | 1 |
| I-1-685 | 6-Cl,8-CH₂CHCH₂ | cyclo-C₃H₅ | 1 |
| I-1-686 | 6-Cl,8-CH₂CHCH₂ | CH₂-cyclo-C₃H₅ | 1 |
| I-1-687 | 6-Cl,8-CH₂CHCH₂ | CH₂CF₃ | 1 |
| I-1-688 | 6-Cl,8-CH₂CCH | CH₃ | 1 |
| I-1-689 | 6-Cl,8-CH₂CCH | CH₂CH₃ | 1 |
| I-1-690 | 6-Cl,8-CH₂CCH | CH₂CHCH₂ | 1 |
| I-1-691 | 6-Cl,8-CH₂CCH | CH₂CCH | 1 |
| I-1-692 | 6-Cl,8-CH₂CCH | cyclo-C₃H₅ | 1 |
| I-1-693 | 6-Cl,8-CH₂CCH | CH₂-cyclo-C₃H₅ | 1 |
| I-1-694 | 6-Cl,8-CH₂CCH | CH₂CF₃ | 1 |
| I-1-695 | 6-Cl,8-cyclo-C₃H₅ | CH₃ | 1 |
| I-1-696 | 6-Cl,8-cyclo-C₃H₅ | CH₂CH₃ | 1 |
| I-1-697 | 6-Cl,8-cyclo-C₃H₅ | CH₂CHCH₂ | 1 |
| I-1-698 | 6-Cl,8-cyclo-C₃H₅ | CH₂CCH | 1 |
| I-1-699 | 6-Cl,8-cyclo-C₃H₅ | cyclo-C₃H₅ | 1 |
| I-1-700 | 6-Cl,8-cyclo-C₃H₅ | CH₂-cyclo-C₃H₅ | 1 |
| I-1-701 | 6-Cl,8-cyclo-C₃H₅ | CH₂CF₃ | 1 |
| I-1-702 | 6-Cl,8-CH₂-cyclo-C₃H₅ | CH₃ | 1 |
| I-1-703 | 6-Cl,8-CH₂-cyclo-C₃H₅ | CH₂CH₃ | 1 |
| I-1-704 | 6-Cl,8-CH₂-cyclo-C₃H₅ | CH₂CHCH₂ | 1 |
| I-1-705 | 6-Cl,8-CH₂-cyclo-C₃H₅ | CH₂CCH | 1 |
| I-1-706 | 6-Cl,8-CH₂-cyclo-C₃H₅ | cyclo-C₃H₅ | 1 |
| I-1-707 | 6-Cl,8-CH₂-cyclo-C₃H₅ | CH₂-cyclo-C₃H₅ | 1 |
| I-1-708 | 6-Cl,8-CH₂-cyclo-C₃H₅ | CH₂CF₃ | 1 |
| I-1-709 | 6-Cl,8-SCH₃ | CH₃ | 1 |
| I-1-710 | 6-Cl,8-SCH₃ | CH₂CH₃ | 1 |
| I-1-711 | 6-Cl,8-SCH₃ | CH₂CHCH₂ | 1 |
| I-1-712 | 6-Cl,8-SCH₃ | CH₂CCH | 1 |
| I-1-713 | 6-Cl,8-SCH₃ | cyclo-C₃H₅ | 1 |
| I-1-714 | 6-Cl,8-SCH₃ | CH₂-cyclo-C₃H₅ | 1 |
| I-1-715 | 6-Cl,8-SCH₃ | CH₂CF₃ | 1 |
| I-1-716 | 6-Cl,8-SCH₂CH₃ | CH₃ | 1 |
| I-1-717 | 6-Cl,8-SCH₂CH₃ | CH₂CH₃ | 1 |
| I-1-718 | 6-Cl,8-SCH₂CH₃ | CH₂CHCH₂ | 1 |
| I-1-719 | 6-Cl,8-SCH₂CH₃ | CH₂CCH | 1 |
| I-1-720 | 6-Cl,8-SCH₂CH₃ | cyclo-C₃H₅ | 1 |
| I-1-721 | 6-Cl,8-SCH₂CH₃ | CH₂-cyclo-C₃H₅ | 1 |
| I-1-722 | 6-Cl,8-SCH₂CH₃ | CH₂CF₃ | 1 |
| I-1-723 | 6-Cl,8-SCH₂CCH | CH₃ | 1 |
| I-1-724 | 6-Cl,8-SCH₂CCH | CH₂CH₃ | 1 |
| I-1-725 | 6-Cl,8-SCH₂CCH | CH₂CHCH₂ | 1 |
| I-1-726 | 6-Cl,8-SCH₂CCH | CH₂CCH | 1 |
| I-1-727 | 6-Cl,8-SCH₂CCH | cyclo-C₃H₅ | 1 |
| I-1-728 | 6-Cl,8-SCH₂CCH | CH₂-cyclo-C₃H₅ | 1 |
| I-1-729 | 6-Cl,8-SCH₂CCH | CH₂CF₃ | 1 |
| I-1-730 | 6-Cl,8-SCH(CH₃)₂ | CH₃ | 1 |
| I-1-731 | 6-Cl,8-SCH(CH₃)₂ | CH₂CH₃ | 1 |
| I-1-732 | 6-Cl,8-SCH(CH₃)₂ | CH₂CHCH₂ | 1 |
| I-1-733 | 6-Cl,8-SCH(CH₃)₂ | CH₂CCH | 1 |
| I-1-734 | 6-Cl,8-SCH(CH₃)₂ | cyclo-C₃H₅ | 1 |
| I-1-735 | 6-Cl,8-SCH(CH₃)₂ | CH₂-cyclo-C₃H₅ | 1 |
| I-1-736 | 6-Cl,8-SCH(CH₃)₂ | CH₂CF₃ | 1 |
| I-1-737 | 6-CH₃,8-CH₃ | CH₃ | 1 |
| I-1-738 | 6-CH₃,8-CH₃ | CH₂CH₃ | 1 |
| I-1-739 | 6-CH₃,8-CH₃ | CH₂CHCH₂ | 1 |
| I-1-740 | 6-CH₃,8-CH₃ | CH₂CCH | 1 |
| I-1-741 | 6-CH₃,8-CH₃ | cyclo-C₃H₅ | 1 |
| I-1-742 | 6-CH₃,8-CH₃ | CH₂-cyclo-C₃H₅ | 1 |
| I-1-743 | 6-CH₃,8-CH₃ | CH₂CF₃ | 1 |
| I-1-744 | 6-CH₃,8-CH₂CH₃ | CH₃ | 1 |
| I-1-745 | 6-CH₃,8-CH₂CH₃ | CH₂CH₃ | 1 |
| I-1-746 | 6-CH₃,8-CH₂CH₃ | CH₂CHCH₂ | 1 |
| I-1-747 | 6-CH₃,8-CH₂CH₃ | CH₂CCH | 1 |
| I-1-748 | 6-CH₃,8-CH₂CH₃ | cyclo-C₃H₅ | 1 |
| I-1-749 | 6-CH₃,8-CH₂CH₃ | CH₂-cyclo-C₃H₅ | 1 |
| I-1-750 | 6-CH₃,8-CH₂CH₃ | CH₂CF₃ | 1 |
| I-1-751 | 6-CH₃,8-(CH₂)₂CH₃ | CH₃ | 1 |
| I-1-752 | 6-CH₃,8-(CH₂)₂CH₃ | CH₂CH₃ | 1 |
| I-1-753 | 6-CH₃,8-(CH₂)₂CH₃ | CH₂CHCH₂ | 1 |
| I-1-754 | 6-CH₃,8-(CH₂)₂CH₃ | CH₂CCH | 1 |
| I-1-755 | 6-CH₃,8-(CH₂)₂CH₃ | cyclo-C₃H₅ | 1 |
| I-1-756 | 6-CH₃,8-(CH₂)₂CH₃ | CH₂-cyclo-C₃H₅ | 1 |
| I-1-757 | 6-CH₃,8-(CH₂)₂CH₃ | CH₂CF₃ | 1 |
| I-1-758 | 6-CH₃,8-CH(CH₃)₂ | CH₃ | 1 |
| I-1-759 | 6-CH₃,8-CH(CH₃)₂ | CH₂CH₃ | 1 |
| I-1-760 | 6-CH₃,8-CH(CH₃)₂ | CH₂CHCH₂ | 1 |
| I-1-761 | 6-CH₃,8-CH(CH₃)₂ | CH₂CCH | 1 |
| I-1-762 | 6-CH₃,8-CH(CH₃)₂ | cyclo-C₃H₅ | 1 |
| I-1-763 | 6-CH₃,8-CH(CH₃)₂ | CH₂-cyclo-C₃H₅ | 1 |
| I-1-764 | 6-CH₃,8-CH(CH₃)₂ | CH₂CF₃ | 1 |
| I-1-765 | 6-CH₃,8-(CH₂)₃CH₃ | CH₃ | 1 |
| I-1-766 | 6-CH₃,8-(CH₂)₃CH₃ | CH₂CH₃ | 1 |
| I-1-767 | 6-CH₃,8-(CH₂)₃CH₃ | CH₂CHCH₂ | 1 |
| I-1-768 | 6-CH₃,8-(CH₂)₃CH₃ | CH₂CCH | 1 |
| I-1-769 | 6-CH₃,8-(CH₂)₃CH₃ | cyclo-C₃H₅ | 1 |
| I-1-770 | 6-CH₃,8-(CH₂)₃CH₃ | CH₂-cyclo-C₃H₅ | 1 |
| I-1-771 | 6-CH₃,8-(CH₂)₃CH₃ | CH₂CF₃ | 1 |
| I-1-772 | 6-CH₃,8-C(CH₃)₃ | CH₃ | 1 |
| I-1-773 | 6-CH₃,8-C(CH₃)₃ | CH₂CH₃ | 1 |
| I-1-774 | 6-CH₃,8-C(CH₃)₃ | CH₂CHCH₂ | 1 |
| I-1-775 | 6-CH₃,8-C(CH₃)₃ | CH₂CCH | 1 |
| I-1-776 | 6-CH₃,8-C(CH₃)₃ | cyclo-C₃H₅ | 1 |
| I-1-777 | 6-CH₃,8-C(CH₃)₃ | CH₂-cyclo-C₃H₅ | 1 |
| I-1-778 | 6-CH₃,8-C(CH₃)₃ | CH₂CF₃ | 1 |
| I-1-779 | 6-CH₃,8-CH₂CHCH₂ | CH₃ | 1 |
| I-1-780 | 6-CH₃,8-CH₂CHCH₂ | CH₂CH₃ | 1 |
| I-1-781 | 6-CH₃,8-CH₂CHCH₂ | CH₂CHCH₂ | 1 |
| I-1-782 | 6-CH₃,8-CH₂CHCH₂ | CH₂CCH | 1 |
| I-1-783 | 6-CH₃,8-CH₂CHCH₂ | cyclo-C₃H₅ | 1 |
| I-1-784 | 6-CH₃,8-CH₂CHCH₂ | CH₂-cyclo-C₃H₅ | 1 |
| I-1-785 | 6-CH₃,8-CH₂CHCH₂ | CH₂CF₃ | 1 |
| I-1-786 | 6-CH₃,8-CH₂CCH | CH₃ | 1 |
| I-1-787 | 6-CH₃,8-CH₂CCH | CH₂CH₃ | 1 |
| I-1-788 | 6-CH₃,8-CH₂CCH | CH₂CHCH₂ | 1 |
| I-1-789 | 6-CH₃,8-CH₂CCH | CH₂CCH | 1 |
| I-1-790 | 6-CH₃,8-CH₂CCH | cyclo-C₃H₅ | 1 |
| I-1-791 | 6-CH₃,8-CH₂CCH | CH₂-cyclo-C₃H₅ | 1 |
| I-1-792 | 6-CH₃,8-CH₂CCH | CH₂CF₃ | 1 |
| I-1-793 | 6-CH₃,8-cyclo-C₃H₅ | CH₃ | 1 |
| I-1-794 | 6-CH₃,8-cyclo-C₃H₅ | CH₂CH₃ | 1 |
| I-1-795 | 6-CH₃,8-cyclo-C₃H₅ | CH₂CHCH₂ | 1 |
| I-1-796 | 6-CH₃,8-cyclo-C₃H₅ | CH₂CCH | 1 |
| I-1-797 | 6-CH₃,8-cyclo-C₃H₅ | cyclo-C₃H₅ | 1 |
| I-1-798 | 6-CH₃,8-cyclo-C₃H₅ | CH₂-cyclo-C₃H₅ | 1 |
| I-1-799 | 6-CH₃,8-cyclo-C₃H₅ | CH₂CF₃ | 1 |
| I-1-800 | 6-CH₃,8-CH₂-cyclo-C₃H₅ | CH₃ | 1 |
| I-1-801 | 6-CH₃,8-CH₂-cyclo-C₃H₅ | CH₂CH₃ | 1 |
| I-1-802 | 6-CH₃,8-CH₂-cyclo-C₃H₅ | CH₂CHCH₂ | 1 |
| I-1-803 | 6-CH₃,8-CH₂-cyclo-C₃H₅ | CH₂CCH | 1 |
| I-1-804 | 6-CH₃,8-CH₂-cyclo-C₃H₅ | cyclo-C₃H₅ | 1 |
| I-1-805 | 6-CH₃,8-CH₂-cyclo-C₃H₅ | CH₂-cyclo-C₃H₅ | 1 |
| I-1-806 | 6-CH₃,8-CH₂-cyclo-C₃H₅ | CH₂CF₃ | 1 |
| I-1-807 | 6-CH₃,8-SCH₃ | CH₃ | 1 |
| I-1-808 | 6-CH₃,8-SCH₃ | CH₂CH₃ | 1 |
| I-1-809 | 6-CH₃,8-SCH₃ | CH₂CHCH₂ | 1 |
| I-1-810 | 6-CH₃,8-SCH₃ | CH₂CCH | 1 |
| I-1-811 | 6-CH₃,8-SCH₃ | cyclo-C₃H₅ | 1 |
| I-1-812 | 6-CH₃,8-SCH₃ | CH₂-cyclo-C₃H₅ | 1 |
| I-1-813 | 6-CH₃,8-SCH₃ | CH₂CF₃ | 1 |
| I-1-814 | 6-CH₃,8-SCH₂CH₃ | CH₃ | 1 |
| I-1-815 | 6-CH₃,8-SCH₂CH₃ | CH₂CH₃ | 1 |
| I-1-816 | 6-CH₃,8-SCH₂CH₃ | CH₂CHCH₂ | 1 |
| I-1-817 | 6-CH₃,8-SCH₂CH₃ | CH₂CCH | 1 |
| I-1-818 | 6-CH₃,8-SCH₂CH₃ | cyclo-C₃H₅ | 1 |
| I-1-819 | 6-CH₃,8-SCH₂CH₃ | CH₂-cyclo-C₃H₅ | 1 |
| I-1-820 | 6-CH₃,8-SCH₂CH₃ | CH₂CF₃ | 1 |
| I-1-821 | 6-CH₃,8-SCH₂CCH | CH₃ | 1 |
| I-1-822 | 6-CH₃,8-SCH₂CCH | CH₂CH₃ | 1 |
| I-1-823 | 6-CH₃,8-SCH₂CCH | CH₂CHCH₂ | 1 |
| I-1-824 | 6-CH₃,8-SCH₂CCH | CH₂CCH | 1 |
| I-1-825 | 6-CH₃,8-SCH₂CCH | cyclo-C₃H₅ | 1 |
| I-1-826 | 6-CH₃,8-SCH₂CCH | CH₂-cyclo-C₃H₅ | 1 |
| I-1-827 | 6-CH₃,8-SCH₂CCH | CH₂CF₃ | 1 |
| I-1-828 | 6-CH₃,8-SCH(CH₃)₂ | CH₃ | 1 |
| I-1-829 | 6-CH₃,8-SCH(CH₃)₂ | CH₂CH₃ | 1 |
| I-1-830 | 6-CH₃,8-SCH(CH₃)₂ | CH₂CHCH₂ | 1 |
| I-1-831 | 6-CH₃,8-SCH(CH₃)₂ | CH₂CCH | 1 |
| I-1-832 | 6-CH₃,8-SCH(CH₃)₂ | cyclo-C₃H₅ | 1 |
| I-1-833 | 6-CH₃,8-SCH(CH₃)₂ | CH₂-cyclo-C₃H₅ | 1 |
| I-1-834 | 6-CH₃,8-SCH(CH₃)₂ | CH₂CF₃ | 1 |
| I-1-835 | 6-OCH₃,8-CH₃ | CH₃ | 1 |
| I-1-836 | 6-OCH₃,8-CH₃ | CH₂CH₃ | 1 |
| I-1-837 | 6-OCH₃,8-CH₃ | CH₂CHCH₂ | 1 |
| I-1-838 | 6-OCH₃,8-CH₃ | CH₂CCH | 1 |
| I-1-839 | 6-OCH₃,8-CH₃ | cyclo-C₃H₅ | 1 |
| I-1-840 | 6-OCH₃,8-CH₃ | CH₂-cyclo-C₃H₅ | 1 |
| I-1-841 | 6-OCH₃,8-CH₃ | CH₂CF₃ | 1 |
| I-1-842 | 6-OCH₃,8-CH₂CH₃ | CH₃ | 1 |
| I-1-843 | 6-OCH₃,8-CH₂CH₃ | CH₂CH₃ | 1 |
| I-1-844 | 6-OCH₃,8-CH₂CH₃ | CH₂CHCH₂ | 1 |
| I-1-845 | 6-OCH₃,8-CH₂CH₃ | CH₂CCH | 1 |
| I-1-846 | 6-OCH₃,8-CH₂CH₃ | cyclo-C₃H₅ | 1 |
| I-1-847 | 6-OCH₃,8-CH₂CH₃ | CH₂-cyclo-C₃H₅ | 1 |
| I-1-848 | 6-OCH₃,8-CH₂CH₃ | CH₂CF₃ | 1 |
| I-1-849 | 6-OCH₃,8-(CH₂)₂CH₃ | CH₃ | 1 |
| I-1-850 | 6-OCH₃,8-(CH₂)₂CH₃ | CH₂CH₃ | 1 |
| I-1-851 | 6-OCH₃,8-(CH₂)₂CH₃ | CH₂CHCH₂ | 1 |
| I-1-852 | 6-OCH₃,8-(CH₂)₂CH₃ | CH₂CCH | 1 |
| I-1-853 | 6-OCH₃,8-(CH₂)₂CH₃ | cyclo-C₃H₅ | 1 |
| I-1-854 | 6-OCH₃,8-(CH₂)₂CH₃ | CH₂-cyclo-C₃H₅ | 1 |
| I-1-855 | 6-OCH₃,8-(CH₂)₂CH₃ | CH₂CF₃ | 1 |
| I-1-856 | 6-OCH₃,8-CH(CH₃)₂ | CH₃ | 1 |
| I-1-857 | 6-OCH₃,8-CH(CH₃)₂ | CH₂CH₃ | 1 |
| I-1-858 | 6-OCH₃,8-CH(CH₃)₂ | CH₂CHCH₂ | 1 |
| I-1-859 | 6-OCH₃,8-CH(CH₃)₂ | CH₂CCH | 1 |
| I-1-860 | 6-OCH₃,8-CH(CH₃)₂ | cyclo-C₃H₅ | 1 |
| I-1-861 | 6-OCH₃,8-CH(CH₃)₂ | CH₂-cyclo-C₃H₅ | 1 |
| I-1-862 | 6-OCH₃,8-CH(CH₃)₂ | CH₂CF₃ | 1 |
| I-1-863 | 6-OCH₃,8-(CH₂)₃CH₃ | CH₃ | 1 |
| I-1-864 | 6-OCH₃,8-(CH₂)₃CH₃ | CH₂CH₃ | 1 |
| I-1-865 | 6-OCH₃,8-(CH₂)₃CH₃ | CH₂CHCH₂ | 1 |
| I-1-866 | 6-OCH₃,8-(CH₂)₃CH₃ | CH₂CCH | 1 |
| I-1-867 | 6-OCH₃,8-(CH₂)₃CH₃ | cyclo-C₃H₅ | 1 |
| I-1-868 | 6-OCH₃,8-(CH₂)₃CH₃ | CH₂-cyclo-C₃H₅ | 1 |
| I-1-869 | 6-OCH₃,8-(CH₂)₃CH₃ | CH₂CF₃ | 1 |
| I-1-870 | 6-OCH₃,8-C(CH₃)₃ | CH₃ | 1 |
| I-1-871 | 6-OCH₃,8-C(CH₃)₃ | CH₂CH₃ | 1 |
| I-1-872 | 6-OCH₃,8-C(CH₃)₃ | CH₂CHCH₂ | 1 |
| I-1-873 | 6-OCH₃,8-C(CH₃)₃ | CH₂CCH | 1 |
| I-1-874 | 6-OCH₃,8-C(CH₃)₃ | cyclo-C₃H₅ | 1 |
| I-1-875 | 6-OCH₃,8-C(CH₃)₃ | CH₂-cyclo-C₃H₅ | 1 |
| I-1-876 | 6-OCH₃,8-C(CH₃)₃ | CH₂CF₃ | 1 |
| I-1-877 | 6-OCH₃,8-CH₂CHCH₂ | CH₃ | 1 |
| I-1-878 | 6-OCH₃,8-CH₂CHCH₂ | CH₂CH₃ | 1 |
| I-1-879 | 6-OCH₃,8-CH₂CHCH₂ | CH₂CHCH₂ | 1 |
| I-1-880 | 6-OCH₃,8-CH₂CHCH₂ | CH₂CCH | 1 |
| I-1-881 | 6-OCH₃,8-CH₂CHCH₂ | cyclo-C₃H₅ | 1 |
| I-1-882 | 6-OCH₃,8-CH₂CHCH₂ | CH₂-cyclo-C₃H₅ | 1 |
| I-1-883 | 6-OCH₃,8-CH₂CHCH₂ | CH₂CF₃ | 1 |
| I-1-884 | 6-OCH₃,8-CH₂CCH | CH₃ | 1 |
| I-1-885 | 6-OCH₃,8-CH₂CCH | CH₂CH₃ | 1 |
| I-1-886 | 6-OCH₃,8-CH₂CCH | CH₂CHCH₂ | 1 |
| I-1-887 | 6-OCH₃,8-CH₂CCH | CH₂CCH | 1 |
| I-1-888 | 6-OCH₃,8-CH₂CCH | cyclo-C₃H₅ | 1 |
| I-1-889 | 6-OCH₃,8-CH₂CCH | CH₂-cyclo-C₃H₅ | 1 |
| I-1-890 | 6-OCH₃,8-CH₂CCH | CH₂CF₃ | 1 |
| I-1-891 | 6-OCH₃,8-cyclo-C₃H₅ | CH₃ | 1 |
| I-1-892 | 6-OCH₃,8-cyclo-C₃H₅ | CH₂CH₃ | 1 |
| I-1-893 | 6-OCH₃,8-cyclo-C₃H₅ | CH₂CHCH₂ | 1 |
| I-1-894 | 6-OCH₃,8-cyclo-C₃H₅ | CH₂CCH | 1 |
| I-1-895 | 6-OCH₃,8-cyclo-C₃H₅ | cyclo-C₃H₅ | 1 |
| I-1-896 | 6-OCH₃,8-cyclo-C₃H₅ | CH₂-cyclo-C₃H₅ | 1 |
| I-1-897 | 6-OCH₃,8-cyclo-C₃H₅ | CH₂CF₃ | 1 |
| I-1-898 | 6-OCH₃,8-CH₂-cyclo-C₃H₅ | CH₃ | 1 |
| I-1-899 | 6-OCH₃,8-CH₂-cyclo-C₃H₅ | CH₂CH₃ | 1 |
| I-1-900 | 6-OCH₃,8-CH₂-cyclo-C₃H₅ | CH₂CHCH₂ | 1 |
| I-1-901 | 6-OCH₃,8-CH₂-cyclo-C₃H₅ | CH₂CCH | 1 |
| I-1-902 | 6-OCH₃,8-CH₂-cyclo-C₃H₅ | cyclo-C₃H₅ | 1 |
| I-1-903 | 6-OCH₃,8-CH₂-cyclo-C₃H₅ | CH₂-cyclo-C₃H₅ | 1 |
| I-1-904 | 6-OCH₃,8-CH₂-cyclo-C₃H₅ | CH₂CF₃ | 1 |
| I-1-905 | 6-OCH₃,8-SCH₃ | CH₃ | 1 |
| I-1-906 | 6-OCH₃,8-SCH₃ | CH₂CH₃ | 1 |
| I-1-907 | 6-OCH₃,8-SCH₃ | CH₂CHCH₂ | 1 |
| I-1-908 | 6-OCH₃,8-SCH₃ | CH₂CCH | 1 |
| I-1-909 | 6-OCH₃,8-SCH₃ | cyclo-C₃H₅ | 1 |
| I-1-910 | 6-OCH₃,8-SCH₃ | CH₂-cyclo-C₃H₅ | 1 |
| I-1-911 | 6-OCH₃,8-SCH₃ | CH₂CF₃ | 1 |
| I-1-912 | 6-OCH₃,8-SCH₂CH₃ | CH₃ | 1 |
| I-1-913 | 6-OCH₃,8-SCH₂CH₃ | CH₂CH₃ | 1 |
| I-1-914 | 6-OCH₃,8-SCH₂CH₃ | CH₂CHCH₂ | 1 |
| I-1-915 | 6-OCH₃,8-SCH₂CH₃ | CH₂CCH | 1 |
| I-1-916 | 6-OCH₃,8-SCH₂CH₃ | cyclo-C₃H₅ | 1 |
| I-1-917 | 6-OCH₃,8-SCH₂CH₃ | CH₂-cyclo-C₃H₅ | 1 |
| I-1-918 | 6-OCH₃,8-SCH₂CH₃ | CH₂CF₃ | 1 |
| I-1-919 | 6-OCH₃,8-SCH₂CCH | CH₃ | 1 |
| I-1-920 | 6-OCH₃,8-SCH₂CCH | CH₂CH₃ | 1 |
| I-1-921 | 6-OCH₃,8-SCH₂CCH | CH₂CHCH₂ | 1 |
| I-1-922 | 6-OCH₃,8-SCH₂CCH | CH₂CCH | 1 |
| I-1-923 | 6-OCH₃,8-SCH₂CCH | cyclo-C₃H₅ | 1 |
| I-1-924 | 6-OCH₃,8-SCH₂CCH | CH₂-cyclo-C₃H₅ | 1 |
| I-1-925 | 6-OCH₃,8-SCH₂CCH | CH₂CF₃ | 1 |
| I-1-926 | 6-OCH₃,8-SCH(CH₃)₂ | CH₃ | 1 |
| I-1-927 | 6-OCH₃,8-SCH(CH₃)₂ | CH₂CH₃ | 1 |
| I-1-928 | 6-OCH₃,8-SCH(CH₃)₂ | CH₂CHCH₂ | 1 |
| I-1-929 | 6-OCH₃,8-SCH(CH₃)₂ | CH₂CCH | 1 |
| I-1-930 | 6-OCH₃,8-SCH(CH₃)₂ | cyclo-C₃H₅ | 1 |
| I-1-931 | 6-OCH₃,8-SCH(CH₃)₂ | CH₂-cyclo-C₃H₅ | 1 |
| I-1-932 | 6-OCH₃,8-SCH(CH₃)₂ | CH₂CF₃ | 1 |
| I-1-933 | 6-CF₃,8-CH₃ | CH₃ | 1 |
| I-1-934 | 6-CF₃,8-CH₃ | CH₂CH₃ | 1 |
| I-1-935 | 6-CF₃,8-CH₃ | CH₂CHCH₂ | 1 |
| I-1-936 | 6-CF₃,8-CH₃ | CH₂CCH | 1 |
| I-1-937 | 6-CF₃,8-CH₃ | cyclo-C₃H₅ | 1 |
| I-1-938 | 6-CF₃,8-CH₃ | CH₂-cyclo-C₃H₅ | 1 |
| I-1-939 | 6-CF₃,8-CH₃ | CH₂CF₃ | 1 |
| I-1-940 | 6-CF₃,8-CH₂CH₃ | CH₃ | 1 |
| I-1-941 | 6-CF₃,8-CH₂CH₃ | CH₂CH₃ | 1 |
| I-1-942 | 6-CF₃,8-CH₂CH₃ | CH₂CHCH₂ | 1 |
| I-1-943 | 6-CF₃,8-CH₂CH₃ | CH₂CCH | 1 |
| I-1-944 | 6-CF₃,8-CH₂CH₃ | cyclo-C₃H₅ | 1 |
| I-1-945 | 6-CF₃,8-CH₂CH₃ | CH₂-cyclo-C₃H₅ | 1 |
| I-1-946 | 6-CF₃,8-CH₂CH₃ | CH₂CF₃ | 1 |
| I-1-947 | 6-CF₃,8-(CH₂)₂CH₃ | CH₃ | 1 |
| I-1-948 | 6-CF₃,8-(CH₂)₂CH₃ | CH₂CH₃ | 1 |
| I-1-949 | 6-CF₃,8-(CH₂)₂CH₃ | CH₂CHCH₂ | 1 |
| I-1-950 | 6-CF₃,8-(CH₂)₂CH₃ | CH₂CCH | 1 |
| I-1-951 | 6-CF₃,8-(CH₂)₂CH₃ | cyclo-C₃H₅ | 1 |
| I-1-952 | 6-CF₃,8-(CH₂)₂CH₃ | CH₂-cyclo-C₃H₅ | 1 |
| I-1-953 | 6-CF₃,8-(CH₂)₂CH₃ | CH₂CF₃ | 1 |
| I-1-954 | 6-CF₃,8-CH(CH₃)₂ | CH₃ | 1 |
| I-1-955 | 6-CF₃,8-CH(CH₃)₂ | CH₂CH₃ | 1 |
| I-1-956 | 6-CF₃,8-CH(CH₃)₂ | CH₂CHCH₂ | 1 |
| I-1-957 | 6-CF₃,8-CH(CH₃)₂ | CH₂CCH | 1 |
| I-1-958 | 6-CF₃,8-CH(CH₃)₂ | cyclo-C₃H₅ | 1 |
| I-1-959 | 6-CF₃,8-CH(CH₃)₂ | CH₂-cyclo-C₃H₅ | 1 |
| I-1-960 | 6-CF₃,8-CH(CH₃)₂ | CH₂CF₃ | 1 |
| I-1-961 | 6-CF₃,8-(CH₂)₃CH₃ | CH₃ | 1 |
| I-1-962 | 6-CF₃,8-(CH₂)₃CH₃ | CH₂CH₃ | 1 |
| I-1-963 | 6-CF₃,8-(CH₂)₃CH₃ | CH₂CHCH₂ | 1 |
| I-1-964 | 6-CF₃,8-(CH₂)₃CH₃ | CH₂CCH | 1 |
| I-1-965 | 6-CF₃,8-(CH₂)₃CH₃ | cyclo-C₃H₅ | 1 |
| I-1-966 | 6-CF₃,8-(CH₂)₃CH₃ | CH₂-cyclo-C₃H₅ | 1 |
| I-1-967 | 6-CF₃,8-(CH₂)₃CH₃ | CH₂CF₃ | 1 |
| I-1-968 | 6-CF₃,8-C(CH₃)₃ | CH₃ | 1 |
| I-1-969 | 6-CF₃,8-C(CH₃)₃ | CH₂CH₃ | 1 |
| I-1-970 | 6-CF₃,8-C(CH₃)₃ | CH₂CHCH₂ | 1 |
| I-1-971 | 6-CF₃,8-C(CH₃)₃ | CH₂CCH | 1 |
| I-1-972 | 6-CF₃,8-C(CH₃)₃ | cyclo-C₃H₅ | 1 |
| I-1-973 | 6-CF₃,8-C(CH₃)₃ | CH₂-cyclo-C₃H₅ | 1 |
| I-1-974 | 6-CF₃,8-C(CH₃)₃ | CH₂CF₃ | 1 |
| I-1-975 | 6-CF₃,8-CH₂CHCH₂ | CH₃ | 1 |
| I-1-976 | 6-CF₃,8-CH₂CHCH₂ | CH₂CH₃ | 1 |
| I-1-977 | 6-CF₃,8-CH₂CHCH₂ | CH₂CHCH₂ | 1 |
| I-1-978 | 6-CF₃,8-CH₂CHCH₂ | CH₂CCH | 1 |
| I-1-979 | 6-CF₃,8-CH₂CHCH₂ | cyclo-C₃H₅ | 1 |
| I-1-980 | 6-CF₃,8-CH₂CHCH₂ | CH₂-cyclo-C₃H₅ | 1 |
| I-1-981 | 6-CF₃,8-CH₂CHCH₂ | CH₂CF₃ | 1 |
| I-1-982 | 6-CF₃,8-CH₂CCH | CH₃ | 1 |
| I-1-983 | 6-CF₃,8-CH₂CCH | CH₂CH₃ | 1 |
| I-1-984 | 6-CF₃,8-CH₂CCH | CH₂CHCH₂ | 1 |
| I-1-985 | 6-CF₃,8-CH₂CCH | CH₂CCH | 1 |
| I-1-986 | 6-CF₃,8-CH₂CCH | cyclo-C₃H₅ | 1 |
| I-1-987 | 6-CF₃,8-CH₂CCH | CH₂-cyclo-C₃H₅ | 1 |
| I-1-988 | 6-CF₃,8-CH₂CCH | CH₂CF₃ | 1 |
| I-1-989 | 6-CF₃,8-cyclo-C₃H₅ | CH₃ | 1 |
| I-1-990 | 6-CF₃,8-cyclo-C₃H₅ | CH₂CH₃ | 1 |
| I-1-991 | 6-CF₃,8-cyclo-C₃H₅ | CH₂CHCH₂ | 1 |
| I-1-992 | 6-CF₃,8-cyclo-C₃H₅ | CH₂CCH | 1 |
| I-1-993 | 6-CF₃,8-cyclo-C₃H₅ | cyclo-C₃H₅ | 1 |
| I-1-994 | 6-CF₃,8-cyclo-C₃H₅ | CH₂-cyclo-C₃H₅ | 1 |
| I-1-995 | 6-CF₃,8-cyclo-C₃H₅ | CH₂CF₃ | 1 |
| I-1-996 | 6-CF₃,8-CH₂-cyclo-C₃H₅ | CH₃ | 1 |
| I-1-997 | 6-CF₃,8-CH₂-cyclo-C₃H₅ | CH₂CH₃ | 1 |
| I-1-998 | 6-CF₃,8-CH₂-cyclo-C₃H₅ | CH₂CHCH₂ | 1 |
| I-1-999 | 6-CF₃,8-CH₂-cyclo-C₃H₅ | CH₂CCH | 1 |
| I-1-1000 | 6-CF₃,8-CH₂-cyclo-C₃H₅ | cyclo-C₃H₅ | 1 |
| I-1-1001 | 6-CF₃,8-CH₂-cyclo-C₃H₅ | CH₂-cyclo-C₃H₅ | 1 |
| I-1-1002 | 6-CF₃,8-CH₂-cyclo-C₃H₅ | CH₂CF₃ | 1 |
| I-1-1003 | 6-CF₃,8-SCH₃ | CH₃ | 1 |
| I-1-1004 | 6-CF₃,8-SCH₃ | CH₂CH₃ | 1 |
| I-1-1005 | 6-CF₃,8-SCH₃ | CH₂CHCH₂ | 1 |
| I-1-1006 | 6-CF₃,8-SCH₃ | CH₂CCH | 1 |
| I-1-1007 | 6-CF₃,8-SCH₃ | cyclo-C₃H₅ | 1 |
| I-1-1008 | 6-CF₃,8-SCH₃ | CH₂-cyclo-C₃H₅ | 1 |
| I-1-1009 | 6-CF₃,8-SCH₃ | CH₂CF₃ | 1 |
| I-1-1010 | 6-CF₃,8-SCH₂CH₃ | CH₃ | 1 |
| I-1-1011 | 6-CF₃,8-SCH₂CH₃ | CH₂CH₃ | 1 |
| I-1-1012 | 6-CF₃,8-SCH₂CH₃ | CH₂CHCH₂ | 1 |
| I-1-1013 | 6-CF₃,8-SCH₂CH₃ | CH₂CCH | 1 |
| I-1-1014 | 6-CF₃,8-SCH₂CH₃ | cyclo-C₃H₅ | 1 |
| I-1-1015 | 6-CF₃,8-SCH₂CH₃ | CH₂-cyclo-C₃H₅ | 1 |
| I-1-1016 | 6-CF₃,8-SCH₂CH₃ | CH₂CF₃ | 1 |
| I-1-1017 | 6-CF₃,8-SCH₂CCH | CH₃ | 1 |
| I-1-1018 | 6-CF₃,8-SCH₂CCH | CH₂CH₃ | 1 |
| I-1-1019 | 6-CF₃,8-SCH₂CCH | CH₂CHCH₂ | 1 |
| I-1-1020 | 6-CF₃,8-SCH₂CCH | CH₂CCH | 1 |
| I-1-1021 | 6-CF₃,8-SCH₂CCH | cyclo-C₃H₅ | 1 |
| I-1-1022 | 6-CF₃,8-SCH₂CCH | CH₂-cyclo-C₃H₅ | 1 |
| I-1-1023 | 6-CF₃,8-SCH₂CCH | CH₂CF₃ | 1 |
| I-1-1024 | 6-CF₃,8-SCH(CH₃)₂ | CH₃ | 1 |
| I-1-1025 | 6-CF₃,8-SCH(CH₃)₂ | CH₂CH₃ | 1 |
| I-1-1026 | 6-CF₃,8-SCH(CH₃)₂ | CH₂CHCH₂ | 1 |
| I-1-1027 | 6-CF₃,8-SCH(CH₃)₂ | CH₂CCH | 1 |
| I-1-1028 | 6-CF₃,8-SCH(CH₃)₂ | cyclo-C₃H₅ | 1 |
| I-1-1029 | 6-CF₃,8-SCH(CH₃)₂ | CH₂-cyclo-C₃H₅ | 1 |
| I-1-1030 | 6-CF₃,8-SCH(CH₃)₂ | CH₂CF₃ | 1 |

The compounds of the formula I are suitable for efficiently controlling nematodes, insects, and arachnids in crop protection. In particular, they are suitable for controlling the following animal pests:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

thrips (Thysanoptera), e.g. Frankliniella fusca, Frankliniella occidentalis, Frankliniella tritici, Scirtothrips citri, Thrips oryzae, Thrips palmi and Thrips tabaci,

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii.

termites (Isoptera), e.g. Calotermes flavicollis, Leucotermes flavipes, Reticulitermes lucifugus und Termes natalensis,

orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus,

Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;

In particular, the compounds of this invention are especially useful for the control of Tetranychus urticae, Panonychus ulmi, Panonychus citri and Brevipalpus phoenicis.

Nematodes, including plant parasitic nematodes and nematodes living in the soil. Plant parasitic nematodes include, such as root knot nematodes, Meloidogyne hapla, Meloidogyne incognita, Meloidogyne javanica, and otherMeloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds I and compositions containing them are especially useful for the control of insects and nematodes.

Moreover, the compounds I and compositions containing them are especially useful for the control of pests selected from the orders Homoptera, Lepidoptera, Diptera, Thysanoptera, and Nematoda.

The compounds of formula (I) may be used to protect growing plants from attack or infestation by insects, arachnids or nematodes by contacting the plant with a pesticidally effective amount of compounds of formula (I).

The insect, arachnid, nematode, plant and/or soil or water in which the plant is growing can be contacted with the present compound(s) or composition(s) by any application method known in the art. As such, “contacting” includes both direct contact (applying the compounds/compositions directly on the insect, arachnid, nematode, and/or plant—typically to the foliage, stem or roots of the plant) and indirect contact (applying the compounds/compositions to the locus of the insect, arachnid, nematode, and/or plant).

Moreover, insects, arachnids or nematodes may be controlled by contacting the target parasite/pest, its food supply or its locus with a pesticidally effective amount of compounds of formula (I). As such, the application may be carried out before or after the infection of the locus, growing crops, or harvested crops by the pest.

“Locus” means a habitat, breeding ground, plant, seed, soil, area, material or environment in which a pest or parasite is growing or may grow.

Exemplary plants and seeds include crop plants and their seeds or nuts, such as vines, wheat, barley, apples, tomatoes, rye, soybeans, oats, rice, maize, lawn, bananas, cotton, coffee, sugar cane, grapevines, fruit species, ornamentals, cucumbers, beans, tomatoes, potatoes and curcubits.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. One of ordinary skill in the art will recognize that the potency and, therefor, a “pesticidally effective amount” can vary for the various compounds/compositions used in the invention.

A pesticidally effective amount of the compositions will also vary according to the prevailing conditions such as desired pesticidal effect and duration, weather, target species, locus, mode of application, and the like. In general, for use in treating crop plants, the rate of application of the compounds and/or compositions of this invention may be in the range of about 0.1 g to about 4000 g per hectare, desirably from about 25 g to about 600 g per hectare as the active ingredient, more desirably from about 50 g to about 500 g per hectare. For use in treating seeds, the typical rate of application is of from about 1 g to about 500 g per kilogram of seeds, desirably from about 2 g to about 300 g per kilogram of seeds, more desirably from about 10 g to about 200 g per kilogram of seeds. Customary application rates in the protection of materials are, for example, from about 0.001 g to about 2 kg, desirably from about 0.005 g to about 1 kg, of active compound per cubic meter of treated material.

The present compounds may be applied formulated or unformulated. Typical formulations contain the active ingredient in a range from about 0.1 ppm to about 10,000 ppm and may also contain a carrier. The carrier may be any agronomically acceptable carrier, including natural and synthetic organic and inorganic ingredients that facilitate dispersion of the composition or compound and contact with the pesticidal target. The carrier may be solid (e.g. clays, synthetic silicates, silica, resins, waxes, kaolin, bentonite, dolomite, calcium carbonate, talc, powdered magnesia, Fuller's earth, gypsum, diatomaceous earth, China clay, and combinations thereof); liquid (e.g. water, aqueous solutions, N-methylpyrrolidone, kerosene, cyclohexanone, methylethyl ketone, acetonitrile, methanol, ethanol, isopropyl alcohol, acetone, butyl cellosolved, 2-ethyl-1hexanol, cyclohexanone, methyl cellulose, polyvinyl alcohol, sodium lignin sulfonates, polymeric alkyl naphthalene sulfonates, sodium naphthalene sulfonate, polymethylene bisnaphthalenesulfonate, sodium N-methyl-N-(long chain acid) laureates, hydrocarbons and other water-immiscible ethers, esters and ketones, and combinations thereof); or a combination of solid and liquid carriers.

The active ingredients can be used as such, in the form of their formulations or the use forms prepared therefrom, e.g. in the form of directly sprayable solutions, powders, suspensions or dispersions, emulsions, oil dispersions, pastes, dusts, materials for spreading, or granules, by means of spraying, atomizing, dusting, scattering or pouring. The use forms depend entirely on the intended purposes. In any case, this is intended to guarantee the finest possible distribution of the active ingredients according to the invention.

The formulations are prepared in a known manner, e.g. by extending the active ingredient with solvents and/or carriers, if desired using emulsifiers and dispersants, it also being possible to use other organic solvents as auxiliary solvents if water is used as the diluent. Auxiliaries which are suitable are essentially: solvents such as aromatics (e.g. xylene), chlorinated aromatics (e.g. chlorobenzenes), paraffins (e.g. mineral oil fractions), alcohols (e.g. methanol, butanol), ketones (e.g. cyclohexanone), amines (e.g. ethanolamine, dimethylformamide) and water, carriers, emulsifiers and dispersants such as lignin-sulfite waste liquors and methylcellulose.

The compositions of the present invention may contain one or more surfactants to increase the biological effectiveness of the active ingredient. Suitable surface active ingredients include surfactants, emulsifying agents, and wetting agents. A wide range of surfactants is available and can be selected readily by those skilled in the art from “The Handbook of Industrial Surfactants,” 2nd Edition, Gower (1997). There is no restriction on the type or chemical class of surfactant that can be used. Nonionic, anionic, cationic and amphoteric types, or combinations of more than one of these types, are all useful in particular situations.

Among nonionic surfactants, exemplary classes include polyoxyethylene alkyl, alkyne, alkynyl or alkylaryl ethers, such as polyoxyethylene primary or secondary alcohols, alkylphenols or acetylenic diols, polyoxyethylene alkyl or alkyne esters, such as ethoxylated fatty acids, sorbitan alkylesters, whether ethoxylated or not, glyceryl alkylesters, sucrose esters, and alkyl polyglycosides. Exemplary anionic surfactant classes include fatty acids, sulfates, sulfonates, and phosphate mono- and diesters of alcohols, alkylphenols, polyoxyethylene alcohols and polyoxyethylene alkylphenols, and carboxylates of polyoxyethylene alcohols and polyoxyethylene alkylphenols. These can be used in their acid form but are more typically used as salts, for example sodium, potassium or ammonium salts.

Cationic surfactants classes include polyoxyethylene tertiary alkylamines or alkenylamines, such as ethoxylated fatty amines, quaternary ammonium surfactants and polyoxyethylene alkyletheramines. Representative specific examples of such cationic surfactants include polyoxyethylene (5) cocoamine, polyoxyethylene (15) tallowamine, distearyldimethylammonium chloride, N-dodecylpyridine chloride and polyoxypropylene (8) ethoxytrimethylammonium chloride. Many cationic quaternary ammonium surfactants of diverse structures are known in the art to be useful in combination with active ingredients and can be used in compositions contemplated herein.

Suitable emulsifying agents and wetting agents include, but are not limited to, ionic and nonionic types such as polyacrylic acid salts, lignosulphonic acid salts, phenolsulphonic or naphthalenesulphonic acids, products of polycondensation of ethylene oxide with fatty alcohols, fatty acids or fatty amines, substituted phenols (especially alkylphenols or arylphenols), sulphonosuccinic acid ester salts, taurine derivatives (especially alkyl taurates), phosphoric esters of alcohols or products of polycondensation of ethylene oxide with phenols, esters of fatty acids with polyhydric alcohols, and derivatives having sulphate, sulphonate and phosphate groups, of the compounds above.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, e.g. benzene, toluene, xylene, paraffin, tetrahydronaphthalene, alkylated naphthalenes or their derivatives, methanol, ethanol, propanol, butanol, chloroform, carbon tetrachloride, cyclohexanol, cyclohexanone, chlorobenzene, isophorone, strongly polar solvents, e.g. dimethylformamide, dimethyl sulfoxide, N-methylpyrrolidone and water.

Powders, materials for scattering and dusts can be prepared by mixing or concomitantly grinding the active substances with a solid carrier.

Granules, e.g. coated granules, impregnated granules and homogeneous granules, can be prepared by binding the active ingredients to solid carriers. Examples of solid carriers are mineral earths, such as silicas, silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, e.g. ammonium sulfate, ammonium phosphate, ammonium nitrate, ureas, and products of vegetable origin, such as cereal meal, tree bark meal, wood meal and nutshell meal, cellulose powders and other solid carriers.

Aqueous use forms can be prepared from emulsion concentrates, pastes or wettable powders (sprayable powders, oil dispersions) by adding water. To prepare emulsions, pastes or oil dispersions, the substances as such or dissolved in an oil or solvent, can be homogenized in water by means of wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within substantial ranges. In general, they are from 0.0001 to 10%, preferably from 0.01 to 1%. The active ingredients are employed in a purity of from 90% to 100%, preferably 95% to 100% (according to NMR spectrum).

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents according to the invention in a weight ratio of 1:10 to 10:1. Mixing the compounds I or the compositions comprising them in the use form as pesticides with other pesticides frequently results in a broader pesticidal spectrum of action.

The following list of pesticides together with which the compounds according to the invention can be used, is intended to illustrate the possible combinations, but not to impose any limitation:

Organophosphates: Acephate, Azinphos-methyl, Chlorpyrifos, Chlorfenvinphos, Diazinon, Dichlorvos, Dicrotophos, Dimethoate, Disulfoton, Ethion, Fenitrothion, Fenthion, Isoxathion, Malathion, Methamidophos, Methidathion, Methyl-Parathion, Mevinphos, Monocrotophos, Oxydemeton-methyl, Paraoxon, Parathion, Phenthoate, Phosalone, Phosmet, Phosphamidon, Phorate, Phoxim, Pirimiphos-methyl, Profenofos, Prothiofos, Sulprophos, Triazophos, Trichlorfon;

Carbamates: Alanycarb, Benfuracarb, Carbaryl, Carbosulfan, Fenoxycarb, Furathiocarb, Indoxacarb, Methiocarb, Methomyl, Oxamyl, Pirimicarb, Propoxur, Thiodicarb, Triazamate;

Pyrethroids: Bifenthrin, Cyfluthrin, Cypermethrin, Deltamethrin, Esfenvalerate, Ethofenprox, Fenpropathrin, Fenvalerate, Cyhalothrin, Lambda-Cyhalothrin, Permethrin, Silafluofen, Tau-Fluvalinate, Tefluthrin, Tralomethrin, Zeta-Cypermethrin;

Arthropod growth regulators: a) chitin synthesis inhibitors: benzoylureas: Chlorfluazuron, Diflubenzuron, Flucycloxuron, Flufenoxuron, Hexaflumuron, Lufenuron, Novaluron, Teflubenzuron, Triflumuron; Buprofezin, Diofenolan, Hexythiazox, Etoxazole, Clofentazine; b) ecdysone antagonists: Halofenozide, Methoxyfenozide, Tebufenozide; c) juvenoids: Pyriproxyfen, Methoprene, Fenoxycarb; d) lipid biosynthesis inhibitors: Spirodiclofen;

Various: Abamectin, Acequinocyl, Amitraz, Azadirachtin, Bifenazate, Cartap, Chlorfenapyr, Chlordimeform, Cyromazine, Diafenthiuron, Dinetofuran, Diofenolan, Emamectin, Endosulfan, Ethiprole, Fenazaquin, Fipronil, Formetanate, Formetanate hydrochloride, Hydramethylnon, Imidacloprid, Indoxacarb, Pyridaben, Pymetrozine, Spinosad, Sulfur, Tebufenpyrad, Thiamethoxam, and Thiocyclam.

Other optional components may be admixed with the present compositions to facilitate the application and/or effectiveness of the active ingredient. To this end, optional components that may be added include antifoaming agents including silicone based antifoaming agents, thickening agents such as fumed silica, antimicrobial agents, antioxidants, buffers, dyes, perfumes, stabilizing agents, and antifreezing agents. Exemplary antifreezing agents include but are not limited to, glycols such as propylene glycol and ethylene glycol, N-methylpyrrolidone, cyclohexanone, and alcohols such as ethanol and methanol.

Compositions of the present invention may be present in any effective formulation, including, but not limited to, a dusting powder or granule, dispersible powder, granule or grain; aqueous dispersion, suspension, paste, or emulsion. As such, the composition may be applied by any effective method including, but not limited to, spraying, atomizing, dusting, spreading or pouring.

Powders, including dusting powders or granules and dispersible powders, granules or grains contain at least one active ingredient and an inert solid extender or carrier, such as kaolin, bentonite, dolomite, calcium carbonate, talc, powdered magnesia, Fuller's earth, gypsum, diatomaceous earth and China clay. Dispersible powders, granules and grains typically also include one or more wetting and dispersing agents, such as surfactants.

The compositions of this invention may be made up of granules comprising 0.5 to 40%, preferably 2 to 30% by weight of the active compounds of this invention as active ingredient; 1 to 20%, preferably 2 to 10% by weight of the surfactant, and 40 to 98.5%, preferably 20 to 96% by weight of solid carrier. Formulated into a dust, the compositions may include 0.5 to 40%, preferably 1 to 35% by weight of the active ingredients; and 99.5 to 60%, preferably 99 to 65% by weight of finely divided solid carrier.

The compositions of this invention may also be formulated into a paste comprising 0.1 to 20%, preferably 1 to 10% by weight of the active ingredient, 1 to 20%, preferably 2 to 10% by weight of surfactant, and 60 to 98.9%, preferably 80 to 97% by weight of paste base. In a wettable powder formulation, the compositions typically includes 5 to 95%, preferably 10 to 50% by weight of the new compounds of this invention as active ingredient, 1 to 20%, preferably 5 to 10% by weight of surfactant, and 4 to 44%, preferably 40 to 85% by weight of solid carrier, the solid carrier being preferably ammonium sulfate.

The aqueous dispersions or emulsions may be prepared by dissolving the active ingredient in an organic solvent optionally containing wetting, dispersing or emulsifying agent(s) and then adding the mixture to water which may also contain wetting, dispersing or emulsifying agents(s). Suitable organic solvents are kerosene, cyclohexanone, methylethyl ketone, acetone, methanol, acetonitrile, and the like. The compositions may also be in the form of liquid preparations for use as dips or sprays which are generally aqueous dispersions or emulsions containing the active ingredient in the presence of one or more wetting agent(s), dispersing agent(s), emulsifying agent(s) or suspending agent(s). Typical liquid solutions include the active ingredient, a carrier, and optionally, a surface active agent. The dilute solutions of the present compositions generally contain about 0.1 to about 50 parts active ingredient, about 0.25 to about 50 parts carrier, and about 0 to about 94 parts surface active agent, all parts being by weight based on the total weight of the composition. Similarly, the concentrated compositions typically include about 40 to about 95 parts active ingredient, about 5 to about 25 parts carrier, and about 0 to about 20 parts surface active agent.

Emulsifications are usually solutions pesticides in water-immiscible or partially water-immiscible solvents as the carrier together with at least one surface active agent. Suitable solvents for the active ingredients of this invention include, but are not limited to, hydrocarbons and water-immiscible ethers, esters or ketones. The emulsification compositions generally contain from 5 to 95%, preferably 20 to 70% by weight of the active compound of this invention as active ingredient, 1 to 40%, preferably 5 to 20% by weight of surfactant, and 4 to 94%, preferably 10 to 75% by weight of liquid carrier.

The present compositions may be prepared in a known manner, for example by homogeneously mixing or grinding the active ingredient(s) with other ingredients. Additional components may be admixed with the composition at any point during the process, including during and/or after any mixing step of the components.

### SYNTHESIS EXAMPLES

The compounds I-1A and I-1B obtained according to the protocols shown in the synthesis examples below together with their physical data are listed in Table I which follows. Compounds I obtained by customary preparation methods described above are listed in Table II which follows.

### Example 1

Preparation of 7-fluoro-10-(4-ethylpiperazine)-dibenzo(b,f)thiepine

A mixture of 1.00 g 7-fluoro-10,11-dihydrodibenzo(b,f)thiepin-10-one, 3.75 g 1-ethylpiperazine and 1.17 g TiCl₄ in 50 ml toluene was heated under reflux for 4 hours. After cooling to 20-25° C., the resulting suspension was filtrated, washed with water, dried with MgSO₄ and concentrated in vacuo to yield 0.79 g of 7-fluoro-10-(4-ethylpiperazine)-dibenzo(b,f)thiepine.

### Example 2

Preparation of 7-fluoro-10-(4-ethylpiperazine)-10,11-dihydrodibenzo(b,f)thiepine

0.50 g 7-Fluoro-10,11-dihydrodibenzo(b,f)thiepin-10-one was dissolved in 50 ml methanol, and 0.30 g NaBH₃CN was added. The solution was acidified with acetic acid until a pH of 5 was reached and stirred at 20-25° C. for 3 days. Methanol was distilled off, the residue was diluted with 10% NaOH solution and extracted 3 times with CH₂Cl₂. The collected organic layers were washed with water, dried with MgSO₄ and concentrated in vacuo to yield 0.50 g of 7-fluoro-10-(4-ethylpiperazine)-10,11-dihydrodibenzo(b,f)thiepine.

TABLE 1

| | |
|---|---|
| 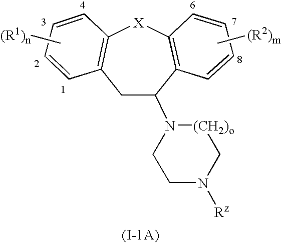 | 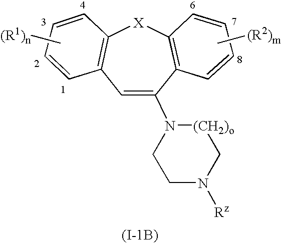 |

| No. | X | (R¹)ₙ | (R²)ₘ | o | R^{z} | Physical data (m.p.[° C.]/¹H-NMR(CDCl₃): δ [ppm]) |
|---|---|---|---|---|---|---|
| I-1A.1 | O | H | 8-Cl | 1 | C₅H₆ | 151-157 |
| I-1A.2 | S | H | 8-SCH₃ | 1 | CH₃ | 188-190 (methane sulfonic acid adduct) |
| I-1A.3 | S | H | 8-Cl | 1 | CH₃ | 203-204 (maleic acid adduct) |
| I-1A.4 | SO₂ | H | 8-Cl | 1 | CH₂CH₃ | 143-146 |
| I-1A.5 | O | H | 8-Cl | 1 | CH₃ | 76-82 |
| I-1A.6 | CH₂ | H | 8-Cl | 1 | CH₃ | 107-110 |
| I-1A.7 | S | H | 8-F | 1 | CH₃ | 140-141 |
| I-1A.8 | S | H | 8-I | 1 | CH₃ | 132-134 |
| I-1A.9 | S | H | H | 1 | CH₃ | 129-131 |
| I-1A.10 | S | H | H | 1 | CH₂CH₃ | 7.6 (d), 7.5 (d), 7.4 (d), 7.3 (t), 7.2 (t), 7.0 (m), |
| | | | | | | 4.0 (m), 3.9 (pt), 3.2 (dd), 2.8 (s), 2.7 (s), 2.5 (s), |
| I-1A.11 | S | 4-Cl | H | 1 | CH₂CH₃ | 7.6 (d), 7.5 (d), 7.3-7.2 (m), 7.1-7.0 (m), 4.1-3.9 (m), |
| | | | | | | 3.1 (d), 2.7 (s), 2.6 (s), 2.5 (s), 2.4 (q), 1.1 (t) |
| I-1A.12 | S | 4-Cl | H | 1 | CH₃ | 7.5 (d), 7.4 (d), 7.2 (m), 7.1 (m), 4.0 (m), 3.2 (d), |
| | | | | | | 2.7 (s), 2.6 (s), 2.5 (s), 2.3 (s) |
| I-1A.13 | S | 2-Cl | H | 1 | CH₂CH₃ | 7.6 (d), 7.5 (d), 7.4 (d), 7.3 (m), 7.2 (t), 7.1 (m), |
| | | | | | | 4.0 (m), 3.9 (pt), 3.1 (d), 2.7 (s), 2.6 (s), 2.5 (s), |
| | | | | | | 2.4 (q), 1.1 (t) |
| I-1A.14 | S | 1-F | 8-Cl | 1 | CH₂CH₃ | 7.7 (s), 7.3-7.2 (m), 7.1-6.9 (m), 4.0 (dd), 3.5 (m), |
| | | | | | | 2.7 (s), 2.5 (s), 2.4 (q), 1.1 (t) |
| I-1A.15 | S | 1-F | 8-Cl | 1 | CH₃ | 7.7 (s), 7.3 (d), 7.2 (d), 7.0 (m), 6.9 (t), 4.0 (dd), |
| | | | | | | 3.5 (m), 2.8 (s), 2.5 (s), 2.3 (s) |
| I-1A.16 | S | 2-F | H | 1 | CH₂CH₃ | 7.6 (d), 7.5 (m), 7.4 (d), 7.2 (t), 7.1 (t), 7.0 (d), |
| | | | | | | 6.8 (t), 4.0-3.9 (m), 3.2 (d), 2.8 (s), 2.7 (s), 2.6 |
| | | | | | | (s), 2.4 (q), 1.1 (t) |
| I-1A.17 | S | 2-F | H | 1 | CH₃ | 7.6 (d), 7.5 (m), 7.4 (d), 7.2 (m), 7.1-6.9 (m), 6.8 |
| | | | | | | (t), 4.0-3.9 (m), 3.1 (d), 2.8 (s), 2.7 (s), 2.5 (s), 2.3 |
| | | | | | | (s) |
| I-1A.18 | S | 1-Cl | H | 1 | CH₂CH₃ | 7.8 (d), 7.4 (d), 7.3 (d), 7.2 (d), 7.1 (t), 7.0 (t), |
| | | | | | | 6.9 (t), 4.0 (m), 3.6 (d), 2.8 (s), 2.5 (s), 2.4 (q), |
| | | | | | | 1.1 (t) |
| I-1A.19 | S | 1-Cl | H | 1 | CH₃ | 7.7 (d), 7.5 (d), 7.4 (d), 7.3 (d), 7.2 (t), 7.1 (t), |
| | | | | | | 7.0 (t), 4.0 (m), 3.6 (d), 2.8 (s), 2.6 (s), 2.3 (s) |
| I-1A.20 | S | 1-Cl | 8-Cl | 1 | CH₂CH₃ | 7.8 (s), 7.3 (d), 7.2 (d), 7.1-7.0 (m), 3.9 (m), 3.7 (m), |
| | | | | | | 2.8 (s), 2.5 (s), 2.4 (u), 1.1 (t) |
| I-1A.21 | S | 1-Cl | 8-Cl | 1 | CH₃ | 7.7 (s), 7.4 (d), 7.3 (d), 7.0 (m), 3.9 (m), 3.7 (m), 2.8 |
| | | | | | | (s), 2.6 (s), 2.4 (s) |
| I-1A.22 | S | 4-F | 8-Cl | 1 | CH₂CH₃ | 7.7 (s), 7.3 (d), 7.2 (m), 7.1 (m), 6.9 (t), 3.9 (m), |
| | | | | | | 3.2 (m), 2.8 (s), 2.7 (s), 2.5 (s), 2.4 (q), 1.1 (t) |
| I-1A.23 | S | 4-F | 8-Cl | 1 | CH₃ | 7.7 (s), 7.4 (d), 7.2 (m), 7.0 (m), 6.9 (t), 3.9 (m), |
| | | | | | | 3.2 (m), 2.7 (s), 2.5 (s), 2.3 (s) |
| I-1A.24 | S | 1-F | H | 1 | CH₂CH₃ | 7.7 (d), 7.4 (d), 7.3 (d), 7.2 (t), 7.1-7.0 (m), 6.9 |
| | | | | | | (t), 4.1 (m), 3.6 (m), 2.8 (m), 2.5 (s), 2.4 (q), 1.1 |
| | | | | | | (t) |
| I-1A.25 | S | 1-F | H | 1 | CH₃ | 7.7 (d), 7.4 (d), 7.3 (d), 7.2 (t), 7.1-7.0 (m), 6.9 |
| | | | | | | (t), 4.1 (dd), 3.7-3.5 (m), 2.7 (m), 2.5 (s), 2.3 (s) |
| I-1A.26 | S | 1,4-F₂ | H | 1 | CH₂CH₃ | 7.6 (d), 7.5 (d), 7.2 (t), 7.1 (t), 7.0-6.8 (m), 4.1 |
| | | | | | | (dd), 3.7 (pt), 3.5 (m), 2.8-2.7 (m), 2.5 (s), 2.4 (q), |
| | | | | | | 1.1 (t) |
| I-1A.27 | S | 1,4-F₂ | H | 1 | CH₃ | 7.6 (d), 7.5 (d), 7.2 (t), 7.1 (t), 7.0-6.8 (m), 4.1 |
| | | | | | | (dd), 3.7 (pt), 3.5 (dd), 2.8-2.7 (m), 2.5 (s), 2.3 (s), |
| I-1A.28 | S | 2-Cl | H | 1 | CH₃ | 7.6 (d), 7.5 (d), 7.4 (d), 7.3 (m), 7.2 (t), 7.1 (t), |
| | | | | | | 4.0 (m), 3.9 (pt), 3.1 (dd), 2.8 (m), 2.7 (m), 2.5 (s), |
| | | | | | | 2.3 (s) |
| I-1A.29 | S | H | H | 1 | CH₂CCH | 7.6 (m), 7.5 (m), 7.4 (d), 7.3-7.1 (m), 4.0 (dd), 3.9 |
| | | | | | | (pt), 3.3 (s), 3.2 (dd), 2.8 (s), 2.7 (s), 2.6 (s), 2.3 |
| | | | | | | (s) |
| I-1A.30 | S | H | H | 1 | CO₂CH₂CH₃ | 7.6 (m), 7.5 (m), 7.4 (d), 7.3-7.0 (m), 4.2 (q), 4.0 |
| | | | | | | (dd), 3.9 (pt), 3.5 (s), 3.4 (s), 3.2 (dd), 2.6 (s), 1.3 |
| | | | | | | (t) |
| I-1A.31 | S | H | H | 1 | CH₂CHCH₂ | 7.6 (m), 7.5 (m), 7.4 (d), 7.3-7.0 (m), 5.9-5.8 (m), |
| | | | | | | 5.2-5.1 (m), 4.0 (dd), 3.9 (pt), 3.2 (dd), 3.0 (d), 2.7 |
| | | | | | | (s), 2.6 (s), 2.5 (s) |
| I-1A.32 | S | H | H | 1 | (CH₂)₂CH₃ | 7.7 (m), 7.5 (m), 7.4 (d), 7.3-7.0 (m), 4.0 (dd), 3.9 |
| | | | | | | (pt), 3.2 (dd), 2.7 (s), 2.5 (s), 2.3-2.2 (m), 1.5 (sx), |
| | | | | | | 0.9 (t) |
| I-1A.33 | S | 4-CF₃ | H | 1 | CH₃ | 7.6 (d), 7.5-7.4 (m), 7.2 (t), 7.1 (t), 7.0 (t), 4.2 |
| | | | | | | (pt), 4.0 (dd), 3.2 (dd), 2.7 (s), 2.6 (s), 2.5 (s), 2.3 |
| | | | | | | (s) |
| I-1A.34 | S | 4-CF₃ | H | 1 | CH₂CH₃ | 7.6 (d), 7.5-7.4 (m), 7.3 (t), 7.2 (t), 7.0 (t), 4.2 |
| | | | | | | (pt), 4.0 (dd), 3.2 (dd), 2.8 (s), 2.7 (s), 2.5 (s), 2.4 |
| | | | | | | (q), 1.1 (t) |
| I-1A.35 | S | H | 7-F | 1 | CH₂CH₃ | 7.6 (dd), 7.5 (d), 7.2 (m), 7.1 (m), 6.8 (t), 3.9 (m), |
| | | | | | | 3.2 (d), 2.7 (s), 2.5 (s), 2.4 (q), 1.1 (t) |
| I-1A.36 | S | H | 7-F | 1 | CH₃ | 7.7 (dd), 7.5 (d), 7.2 (m), 7.1 (m), 6.8 (m), 3.9 (m), |
| | | | | | | 3.1 (d), 2.7 (s), 2.5 (s), 2.3 (s) |
| I-1A.37 | S | H | 8-CH₃ | 1 | CH₃ | 108-112 |
| I-1A.38 | S | H | 8-CH₃ | 1 | CH₂CH₃ | 7.5 (d), 7.4 (s), 7.3 (s), 7.25 (d), 7.2 (t), 7.1 (t), |
| | | | | | | 6.9 (d), 4.0 (m), 3.9 (pt), 3.2 (d), 2.7 (s), 2.6 (m), |
| | | | | | | 2.5 (s), 2.4 (q), 2.3 (s), 1.1 (t) |
| 1-1A.39 | S | H | 6,8-(CH₃)₂ | 1 | CH₂CF₃ | 7.4 (d), 7.3-7.2 (m), 7.1 (t), 6.8 (s), 4.2 (dd), 3.7 |
| | | | | | | (pt), 3.2 (dd), 2.9 (q), 2.7-2.5 (m), 2.4 (s), 2.2 (s) |
| I-1A.40 | S | H | 8-Br | 1 | CH₂CH₃ | 7.8 (s), 7.5 (d), 7.3-7.1 (m), 3.9 (m), 3.2 (d), 2.7 |
| | | | | | | (s), 2.5 (s), 2.4 (q), 1.1 (t) |
| I-1A.41 | S | H | 6,8-(CH₃)₂ | 1 | CH₃ | 7.5 (d), 7.2-7.1 (m), 7.0 (t), 6.9 (s), 4.2 (dd), 3.7 |
| | | | | | | (pt), 3.3 (dd), 2.8 (s), 2.6 (s), 2.5 (s), 2.3 (s), 2.2 |
| | | | | | | (s) |
| I-1A.42 | S | H | H | 1 | CH₂CClF₂ | 7.6 (d), 7.5 (d), 7.4 (d), 7.3-7.0 (m), 4.0-3.9 (m), |
| | | | | | | 3.1-3.0 (m), 2.8-2.7 (m) |
| I-1A.43 | S | H | H | 1 | CH₂CCl₃ | 7.7 (d), 7.5 (d), 7.4 (d), 7.3-7.0 (m), 4.0 (m), 3.9 |
| | | | | | | (m), 3.3 (s), 3.1 (dd), 2.9-2.8 (m), 2.7 (m) |
| I-1A.44 | S | H | H | 1 | CH₂CF₃ | 7.6 (d), 7.5 (d), 7.4 (d), 7.3-7.0 (m), 4.0 (m), 3.9 |
| | | | | | | (m), 3.1 (dd), 2.9 (q), 2.6 (m) |
| I-1A.45 | S | H | 8-Br | 1 | CH₂CF₃ | 7.8 (s), 7.5 (d), 7.2 (m), 7.15 (d), 7.1 (t), 3.9 (m), |
| | | | | | | 3.1 (d), 2.9 (q), 2.6 (s) |
| I-1A.46 | S | H | 8-CH₃ | 1 | CH₂CF₃ | 89 |
| I-1A.47 | S | H | 6,8-(CH₃)₂ | 1 | CH₂CH₃ | 7.4 (d), 7.3 (s), 7.2-7.1 (m), 7.0 (t), 6.8 (s), 4.2 |
| | | | | | | (dd), 3.7 (pt), 3.3 (dd), 2.8 (s), 2.6 (s), 2.5 (s), 2.4 |
| | | | | | | (q), 2.2 (s), 1.1 (t) |
| I-1A.48 | S | H | 8-C(CH₃)₃ | 1 | CH₃ | 127 |
| I-1A.49 | S | H | 8-C(CH₃)₃ | 1 | CH₂CF₃ | 113 |
| I-1A.50 | S | H | 8-C(CH₃)₃ | 1 | CH₂CCl₃ | 150-152 |
| I-1A.51 | S | H | 8-CH₃ | 1 | CH₂CCl₃ | 124 |
| I-1A.52 | S | H | 6,8-(CH₃)₂ | 1 | CH₂CCl₃ | 131 |
| I-1A.53 | S | H | 8-Br | 1 | CH₃ | 111 |
| I-1A.54 | S | H | 8-C(CH₃)₃ | 1 | CH₂CH₃ | 7.7 (s), 7.5 (d), 7.4 (d), 7.3 (d), 7.2 (t), 7.1 (t), |
| | | | | | | 4.0 (m), 3.8 (pt), 3.2 (dd), 2.7 (s), 2.5 (s), 2.4 (q), |
| | | | | | | 1.2 (s), 1.1 (t) |
| I-1A.55 | S | H | 8-CH₂CH₃ | 1 | CH₂CF₃ | 7.5 (d), 7.45 (s), 7.4 (d), 7.3 (d), 7.2 (t), 7.1 (t), |
| | | | | | | 6.9 (d), 4.0 (m), 3.9 (pt), 3.2 (dd), 2.9 (q), 2.8-2.7 |
| | | | | | | (m), 2.5 (q), 1.2 (t) |
| I-1A.56 | S | H | 8-CH(CH₃)₂ | 1 | CH₃ | 7.5 (m), 7.3 (d), 7.25 (t), 7.2 (t), 7.0 (t), 6.8 (d), |
| | | | | | | 4.0 (m), 3.9 (pt), 3.2 (dd), 2.8 (hpt), 2.7 (m), 2.4 |
| | | | | | | (s), 2.3 (s), 1.2 (d) |
| I-1A.57 | S | H | 8-CH(CH₃)₂ | 1 | CH₂CH₃ | 7.5 (m), 7.3 (d), 7.25 (t), 7.2 (t), 7.0 (t), 6.9 (d), |
| | | | | | | 4.0 (m), 3.9 (pt), 3.2 (dd), 2.8 (hpt), 2.7-2.6 (m), 2.4 |
| | | | | | | (q), 1.2 (d), 1.1 (t) |
| I-1A.58 | S | H | 8-CH(CH₃)₂ | 1 | CH₂CF₃ | 7.5 (d), 7.4 (d), 7.3-7.2 (m), 7.1 (t), 6.9 (d), 4.0 |
| | | | | | | (m), 3.9 (pt), 3.2 (dd), 2.9 (hpt), 2.8-2.6 (m), 1.2 (d) |
| I-1A.59 | S | H | 6-Cl | 1 | CH₂CF₃ | 7.6 (d), 7.4 (d), 7.3 (m), 7.2-7.1 (m), 4.1 (dd), 3.8 |
| | | | | | | (pt), 3.2 (dd), 3.0 (q), 2.8-2.6 (m) |
| I-1A.60 | S | H | 6-Cl | 1 | CH₂CH₃ | 7.6 (d), 7.5 (d), 7.2 (m), 7.1-7.0 (m), 4.0 (dd), 3.8 |
| | | | | | | (pt), 3.2 (dd), 2.8 (m), 2.7 (m), 2.5 (m), 2.4 (q), 1.1 |
| | | | | | | (t) |
| I-1A.61 | S | H | 6-Cl | 1 | CH₂CCl₃ | 7.5 (d), 7.4 (d), 7.2 (m), 7.1-7.0 (m), 4.0 (dd), 3.8 |
| | | | | | | (pt), 3.3 (s), 3.2 (dd), 2.9-2.8 (m), 2.7-2.6 (m) |
| I-1A.62 | S | H | 6-F | 1 | CH₂CH₃ | 7.6 (d), 7.4 (d), 7.3 (m), 7.1 (m), 6.9 (t), 4.0 (dd), |
| | | | | | | 3.9 (pt), 3.2 (dd), 2.8 (m), 2.7 (m), 2.5 (s), 2.4 (q), |
| | | | | | | 1.1 (t) |
| I-1A.63 | S | H | 6-F | 1 | CH₃ | 7.6 (d), 7.4 (d), 7.3 (m), 7.1 (m), 6.9 (t), 4.0 (dd), |
| | | | | | | 3.9 (pt), 3.2 (dd), 2.8 (m), 2.7 (m), 2.4 (s), 2.3 (s) |
| I-1A.64 | S | H | 6-F | 1 | CH₂CCl₃ | 7.6 (d), 7.4 (d), 7.3 (m), 7.1 (m), 6.9 (t), 4.1-3.9 |
| | | | | | | (m), 3.3 (s), 3.2 (dd), 3.0-2.9 (m), 2.8-2.6 (m) |
| I-1A.65 | S | H | 6-F | 1 | CH₂CF₃ | 7.6 (d), 7.4 (d), 7.3 (m), 7.1 (m), 6.9 (t), 4.0 (m), |
| | | | | | | 3.9 (pt), 3.2 (dd), 2.9 (q), 2.7-2.6 (m) |
| I-1A.66 | S | H | 6-OCH₃ | 1 | CH₃ | 7.6 (d), 7.2 (m), 7.1 (m), 6.7 (d), 4.1 (dd), 3.9 (s), |
| | | | | | | 3.8 (pt), 3.2 (dd), 2.7-2.6 (m), 2.4 (s), 2.2 (s) |
| I-1A.67 | S | H | 8-CH₂CH₃ | 1 | CH₂CH₃ | 7.5 (d), 7.4 (s), 7.3 (d), 7.25 (m), 7.2 (t), 7.1 (t), |
| | | | | | | 6.8 (d), 4.1 (m), 3.9 (pt), 3.2 (d), 2.8 (s), 2.7 (s), |
| | | | | | | 2.5 (q), 2.4 (q), 1.2 (t), 1.1 (t) |
| I-1A.68 | S | H | 7-CH₃ | 1 | CH₃ | 131 |
| I-1A.69 | S | H | 7-CH₃ | 1 | CH₂CH₃ | 7.5 (t), 7.3-7.2 (m), 7.1 (t), 6.9 (d), 4.0-3.9 (m), 3.2 |
| | | | | | | (d), 2.8 (m), 2.4 (s), 2.3 (q), 2.2 (s), 1.1 (t) |
| I-1A.70 | S | H | 7-Br | 1 | CH₃ | 118 |
| I-1A.71 | S | H | 7-Br | 1 | CH₂CH₃ | 7.5 (m), 7.2 (m), 7.1 (t), 3.9 (m), 3.2 (m), 2.7 (s), |
| | | | | | | 2.5 (s), 2.4 (q), 1.1 (t) |
| I-1A.72 | S | H | 7-Cl | 1 | CH₂CH₃ | 7.6 (d), 7.5 (d), 7.4 (s), 7.2 (m), 7.1 (m), 3.9 (m), |
| | | | | | | 3.2 (d), 2.7 (s), 2.5 (s), 2.4 (q), 1.1 (t) |
| I-1A.73 | S | H | 7-OCH₃ | 1 | CH₂CH₃ | 7.5 (m), 7.2 (m), 7.1 (t), 6.9 (s), 6.7 (d), 3.9 (m), |
| | | | | | | 3.7 (s), 3.2 (d), 2.7 (s), 2.5 (s), 2.4 (q), 1.1 (t) |
| I-1A.74 | CH₂ | H | 8-CH₂CH₃ | 1 | CH₂CH₃ | 7.2 (s), 7.1-7.0 (m), 4.7 (d), 3.7 (dd), 3.5 (d), 3.3 |
| | | | | | | (dd), 3.1 (dd), 2.7-2.5 (m), 2.5 (q), 2.3 (q), 1.2 (t), |
| | | | | | | 1.1 (t) |
| I-1A.75 | CH₂ | H | 8-CH₂CH₃ | 1 | CH₃ | 7.2 (s), 7.1-7.0 (m), 4.7 (d), 3.7 (dd), 3.6 (d), 3.4 |
| | | | | | | (dd), 3.1 (dd), 2.7-2.5 (m), 2.5 (q), 2.4 (s), 2.2 (s), |
| | | | | | | 1.1 (t) |
| I-1A.76 | S | H | 8-Cl | 2 | CH₃ | 7.8 (s), 7.5 (d), 7.3-7.2 (m), 7.1 (t), 7.0 (d), 4.0-3.9 |
| | | | | | | (m), 3.1 (d), 3.0-2.8 (m), 2.7 (t), 2.6 (m), 2.5 (m), |
| | | | | | | 2.4 (s), 1.9-1.7 (m) |
| I-1B.1 | C═O | 2-Cl | H | 1 | CH₃ | 205-208 (fumaric acid adduct) |
| I-1B.2 | S | H | 8-NH₂ | 1 | CH₃ | 101-102 |
| I-1B.3 | S | H | 8-Cl | 1 | CH₂CH₃ | 100-105 |
| I-1B.4 | S | 2-Cl | H | 1 | CH₃ | 7.6 (m), 7.5 (m), 7.4 (d), 7.3-7.2 (m), 7.1 (d), 6.2 |
| | | | | | | s( ), 3.0 (m), 2.6 (s), 2.5 (s), 2.3 (s) |
| I-1B.5 | S | 2-Cl | H | 1 | CH₂CH₃ | 7.6 (m), 7.5 (m), 7.4 (d), 7.3-7.2 (m), 7.1 (d), 6.2 |
| | | | | | | (s), 3.0 (s), 2.6-2.4 (m), 1.1 (t) |
| I-1B.6 | S | 1-Cl | 8-Cl | 1 | CH₂CH₃ | 7.6 (s), 7.5 (d), 7.4 (d), 7.2 (m), 7.1 (t), 6.3 (s), |
| | | | | | | 3.0 (s), 2.7-2.4 (m), 1.1 (t) |
| I-1B.7 | S | 2-F | H | 1 | CH₂CH₃ | 7.6 (m), 7.5 (m), 7.4 (d), 7.3-7.2 (m), 6.9 (d), 6.8 |
| | | | | | | (m), 6.2 (s), 3.0 (m), 2.6-2.4 (m), 1.1 (t) |
| I-1B.8 | S | 4-F | 8-Cl | 1 | CH₂CH₃ | 7.6 (s), 7.5 (d), 7.2 (d), 7.1 (m), 7.0 (d), 6.9 (t), |
| | | | | | | 6.3 (s), 3.0 (s), 2.6-2.4 (m), 1.1 (t) |
| I-1B.9 | S | 1-Cl | 8-Cl | 1 | CH₃ | 7.6 (s), 7.5 (d), 7.4 (d), 7.2 (d), 7.1 (t), 6.3 (s), |
| | | | | | | 3.0 (s), 2.6-2.4 (m), 2.3 (s) |
| I-1B.10 | S | 2-F | H | 1 | CH₃ | 7.6 (d), 7.5 (t), 7.4 (m), 7.3 (m), 6.9 (d), 6.8 (t), |
| | | | | | | 6.2 (s), 3.0 (s), 2.6-2.5 (m), 2.3 (s) |
| I-1B.11 | S | 4-F | 8-Cl | 1 | CH₃ | 7.6 (s), 7.5 (d), 7.2 (d), 7.1 (m), 7.0 (d), 6.9 (t), |
| | | | | | | 6.3 (s), 3.0 (s), 2.6-2.5 (m), 2.4 (s) |
| I-1B.12 | S | 1-Cl | H | 1 | CH₃ | 7.6 (m), 7.5 (m), 7.3 (d), 7.2 (m), 7.1 (t), 6.3 (s), |
| | | | | | | 3.0 (s), 2.6-2.5 (m), 2.3 (s) |
| I-1B.13 | S | 1-Cl | H | 1 | CH₂CH₃ | 7.6 (m), 7.5 (m), 7.4 (d), 7.3-7.2 (m), 7.1 (t), 6.3 |
| | | | | | | (s), 3.1 (m), 2.6 (s), 2.5 (m), 2.4 (q), 1.1 (t) |
| I-1B.14 | S | 4-Cl | H | 1 | CH₃ | 7.6 (m), 7.3-7.2 (m), 7.1 (t), 6.3 (5), 3.0 (s), 2.6 |
| | | | | | | (s), 2.5 (s), 2.3 (s) |
| I-1B.15 | S | 4-Cl | H | 1 | CH₂CH₃ | 7.6 (m), 7.3-7.2 (m), 7.1 (t), 6.3 (s), 3.0 (s), 2.6 |
| | | | | | | (s), 2.5 (s), 2.4 (q), 1.1 (t) |
| I-1B.16 | S | 4-F | H | 1 | CH₃ | 7.6 (m), 7.3 (m), 7.1 (m), 7.0 (d), 6.9 (t), 6.3 (s), |
| | | | | | | 3.0 (s), 2.6 (s), 2.5 (s), 2.3 (s) |
| I-1B.17 | S | 4-F | H | 1 | CH₂CH₃ | 7.6 (m), 7.4-7.3 (m), 7.2-7.1 (m), 7.0 (d), 6.9 (t), 6.3 |
| | | | | | | (s), 3.1 (s), 2.6 (s), 2.5 (s), 2.4 (q), 1.1 (t) |
| I-1B.18 | S | 1-F | H | 1 | CH₃ | 7.6 (m), 7.5 (m), 7.3-7.2 (m), 7.1 (m), 6.9 (t), 6.3 |
| | | | | | | (s), 3.1 (s), 2.6 (s), 2.5 (m), 2.3 (s) |
| I-1B.19 | S | 1-F | H | 1 | CH₂CH₃ | 7.6 (m), 7.5 (m), 7.3 (m), 7.2 (d), 7.1 (m), 6.9 (t), |
| | | | | | | 6.3 (s), 3.1 (s), 2.6 (s), 2.5 (s), 2.4 (q), 1.1 (t) |
| I-1B.20 | S | 2-F | 8-Cl | 1 | CH₂CH₃ | 7.6 (s), 7.5-7.4 (m), 7.3 (m), 7.2 (d), 6.9 (d), 6.8 |
| | | | | | | (t), 6.2 (s), 3.1 (s), 2.6 (s), 2.5 (s), 2.4 (q), 1.1 |
| | | | | | | (t) |
| I-1B.21 | S | 1-F | 8-Cl | 1 | CH₃ | 7.6 (s), 7.5 (d), 7.3-7.0 (m), 6.9 (t), 6.3 (s), 3.1 |
| | | | | | | (s), 2.6 (s), 2.5 (s), 2.3 (s) |
| I-1B.22 | S | 1-F | 8-Cl | 1 | CH₂CH₃ | 7.6 (s), 7.5 (d), 7.3-7.1 (m), 6.9 (t), 6.3 (s), 3.1 |
| | | | | | | (s), 2.6 (s), 2.5 (s), 2.4 (q), 1.1 (t) |
| I-1B.23 | S | 1,4-F₂ | H | 1 | CH₃ | 7.6 (m), 7.3 (m), 6.8 (m), 6.3 (s), 3.1 (s), 2.6 (s), |
| | | | | | | 2.5 (s), 2.3 (s) |
| I-1B.24 | S | 1,4-F₂ | H | 1 | CH₂CH₃ | 7.6 (m), 7.3-7.1 (m), 6.9 (m), 6.2 (s), 3.1 (m), 2.6 |
| | | | | | | (s), 2.5 (m), 2.4 (q), 1.1 (t) |
| I-1B.25 | S | 4-CF₃ | H | 1 | CH₃ | 7.6 (m), 7.5 (d), 7.4 (d), 7.3 (m), 7.2 (t), 6.3 (s), |
| | | | | | | 3.1 (m), 2.6 (s), 2.5 (m), 2.4 (s) |
| I-1B.26 | S | 4-CF₃ | H | 1 | CH₂CH₃ | 7.6 (m), 7.5 (d), 7.4 (d), 7.3 (m), 7.2 (t), 6.3 (s), |
| | | | | | | 3.1 (m), 2.6 (s), 2.5 (m), 2.4 (q), 1.1 (t) |
| I-1B.27 | S | H | 7-F | 1 | CH₂CH₃ | 7.6 (dd), 7.4 (d), 7.3-7.1 (m), 6.9 (t), 6.3 (s), 3.0 |
| | | | | | | (s), 2.6 (s), 2.4 (q), 1.1 (t) |
| I-1B.28 | S | H | 7-F | 1 | CH₃ | 7.6 (dd), 7.5 (d), 7.3-7.1 (m), 7.0 (t), 6.3 (s), 3.1 |
| | | | | | | (s), 2.5 (s), 2.4 (s) |
| I-1B.29 | S | H | 7-F | 1 | CH₂CCl₃ | 7.6 (dd), 7.4 (d), 7.3-7.1 (m), 7.0 (t), 6.3 (s), 3.3 |
| | | | | | | (s), 3.0 (s) |
| I-1B.30 | S | H | 7-F | 1 | CH₂CF₃ | 7.6 (dd), 7.5 (d), 7.3-7.1 (m), 7.0 (t), 6.3 (s), |
| | | | | | | 3.1-2.9 (m), 2.8 (m) |
| I-1B.31 | S | H | 8-Br | 1 | CH₂CH₃ | 109-113 |
| I-1B.32 | S | H | 8-Br | 1 | CH₃ | 112-114 |
| I-1B.33 | S | H | 8-CH₃ | 1 | CH₂CH₃ | 7.5-7.4 (m), 7.3-7.1 (m), 6.3 (s), 3.1 (s), 2.6 (s), 2.5 |
| | | | | | | (q), 2.2 (s), 1.1 (t), |
| I-1B.34 | S | H | 8-CH₃ | 1 | CH₃ | 7.5-7.4 (m), 7.3-7.1 (m), 6.3 (s), 3.1 (s), 2.6 (m), 2.4 |
| | | | | | | (s), 2.3 (s) |
| I-1B.35 | S | H | 8-Br | 1 | CH₂CF₃ | 144-145 |
| I-1B.36 | S | H | 8-CH₃ | 1 | CH₂CF₃ | 142-143 |
| I-1B.37 | S | H | 6,8-(CH₃)₂ | 1 | CH₂CF₃ | 119-120 |
| I-1B.38 | S | H | 6,8-(CH₃)₂ | 1 | CH₂ | 7.5 (d), 7.3-7.1 (m), 6.3 (s), 3.0 (s), 2.6 (s), 2.5 |
| | | | | | | (s), 2.4 (s) |
| I-1B.39 | S | H | 6,8-(CH₃)₂ | 1 | CH₂CH₃ | 7.5 (d), 7.3-7.0 (m), 6.3 (s), 3.0 (s), 2.6 (s), 2.55 |
| | | | | | | (s), 2.5 (q), 2.2 (s), 1.1 (t) |
| I-1B.40 | S | H | 8-CH₂CH₃ | 1 | CH₂CH₃ | 7.5 (m), 7.2 (m), 7.15 (t), 7.1 (m), 6.3 (s), 3.0 (s), |
| | | | | | | 2.6 (m), 2.5 (m), 1.2 (t), 1.1 (t) |
| I-1B.41 | S | H | H | 1 | CH(CH₃)₂ | 7.6 (m), 7.5 (m), 7.4 (d), 7.3-7.0 (m), 6.3 (s), 3.0 |
| | | | | | | (s), 2.7 (hpt), 2.6 (s), 1.1 (d) |
| I-1B.42 | S | H | H | 1 | CH₂CF₃ | 7.6 (m), 7.5 (m), 7.4 (d), 7.3-7.1 (m), 6.3 (s), 3.1-3.0 |
| | | | | | | (m), 2.9 (s), 2.8 (s) |
| I-1B.43 | S | H | H | 1 | CH₂CCl₃ | 7.6 (m), 7.5 (m), 7.4 (d), 7.3-7.1 (m), 6.3 (s), 3.2 |
| | | | | | | (s), 3.0 (s) |
| I-1B.44 | S | H | H | 1 | CH₂CHCH | 7.6 (m), 7.5 (m), 7.4 (d), 7.3-7.1 (m), 6.3 (s), 6.0-5.8 |
| | | | | | | (m), 5.2-5.1 (m), 3.1-3.0 (m), 2.7-2.5 (m) |
| I-1B.45 | S | H | H | 1 | CH₂CH₃ | 7.8 (m), 7.6 (m), 7.5 (d), 7.3-7.1 (m), 6.3 (s), 3.0 |
| | | | | | | (s), 2.7-2.6 (m), 2.5 (q), 1.2 (t) |
| I-1B.46 | S | H | H | 1 | CO₂CH₂CH₃ | 7.7 (m), 7.6 (m), 7.5 (d), 7.3-7.1 (m), 6.3 (s), 4.2 (q), |
| | | | | | | 3.7-3.5 (m), 3.0 (s), 1.3 (t) |
| I-1B.47 | C═O | 2-Cl | H | 1 | CH₂CH₃ | 8.0 (d), 7.9 (d), 7.8 (d), 7.6 (m), 7.5 (m), 7.4 (s), |
| | | | | | | 7.3 (d), 7.2 (m), 6.3 (s), 3.0 (s), 2.6 (s), 2.5 (q), |
| | | | | | | 1.1 (t) |
| I-1B.48 | S | H | 8-C(CH₃)₃ | 1 | CH₂CCl₃ | 169 |
| I-1B.49 | S | H | 6,8-(CH₃)₂ | 1 | CH₂CCl₃ | 167 |
| I-1B.50 | S | H | 8-C(CH₃)₃ | 1 | CH₂CF₃ | 159 |
| I-1B.51 | S | H | 8-C(CH₃)₃ | 1 | CH₃ | 172 |
| I-1B.52 | S | H | 8-C(CH₃)₃ | 1 | CH₂CH₃ | 85 |
| I-1B.53 | S | H | 8-CH(CH₃)₂ | 1 | CH₃ | 105-107 |
| I-1B.54 | S | H | 8-CH(CH₃)₂ | 1 | CH₂CH₃ | 7.5-7.4 (m), 7.3-7.1 (m), 6.3 (s), 3.1 (s), 2.9 (hpt), |
| | | | | | | 2.6 (s), 2.5 (m), 1.2 (d), 1.1 (t) |
| I-1B.55 | S | H | 8-CH₂CH₃ | 1 | CH₂CF₃ | 7.5-7.4 (m), 7.3-7.1 (m), 6.3 (s), 3.0 (m), 2.8 (m), 2.6 |
| | | | | | | (q), 1.2 (t) |
| I-1B.56 | S | H | 8-CH(CH₃)₂ | 1 | CH₂CCl₃ | 7.5 (s), 7.4-7.3 (m), 7.2 (d), 7.1-7.0 (m), 6.3 (s), 3.3 |
| | | | | | | (s), 3.0 (s), 2.8 (hpt), 1.2 (d) |
| I-1B.57 | S | H | 8-CH₃ | 1 | CH₂CCl₃ | 178 |
| I-1B.58 | S | H | 6-OCH₃ | 1 | CH₂CH₃ | 7.5 (d), 7.3-7.1 (m), 6.9 (d), 6.3 (s), 3.9 (s), 3.0 |
| | | | | | | (s), 1.2 (t) |
| I-1B.59 | S | H | 6-OCH₃ | 1 | CH₃ | 7.5 (d), 7.3 (t), 7.2 (t), 7.1 (t), 6.8 (d), 6.3 (s), |
| | | | | | | 3.9 (s), 3.0 (s), 2.6 (s), 2.5 (s), 2.3 (s) |
| I-1B.60 | S | H | 6-Cl | 1 | CH₂CH₃ | 7.6 (dd), 7.4 (d), 7.3-7.1 (m), 6.3 (s), 3.0 (m), |
| | | | | | | 2.6-2.4 (m), 1.1 (t) |
| I-1B.61 | S | H | 6-Cl | 1 | CH₂CF₃ | 7.6 (d), 7.5 (d), 7.4 (d), 7.3-7.1 (m), 6.3 (s), 3.0 |
| | | | | | | (m), 2.9 (m), 2.8 (m), 2.3 (s) |
| I-1B.62 | S | H | 6-Cl | 1 | CH₂CCl₃ | 7.6 (m), 7.4 (s), 7.2-7.1 (m), 6.4 (s), 3.3 (d), 3.1-2.9 |
| | | | | | | (m) |
| I-1B.63 | S | H | 6-Cl | 1 | CH₃ | 7.6 (m), 7.4 (d), 7.3-7.1 (m), 6.3 (s), 3.0 (s), 2.6 |
| | | | | | | (s), 2.5 (s), 2.3 (s) |
| I-1B.64 | S | H | 6-F | 1 | CH₂CH₃ | 7.6 (d), 7.4 (d), 7.3-7.1 (m), 6.4 (s), 3.0 (s), 2.6 |
| | | | | | | (m), 2.5 (q), 1.2 (t) |
| I-1B.65 | S | H | 6-F | 1 | CH₃ | 7.6 (d), 7.4 (d), 7.3-7.2 (m), 7.1 (t), 6.3 (s), 3.0 |
| | | | | | | (s), 2.6-2.5 (m), 2.3 (s) |
| I-1B.66 | S | H | 6-F | 1 | CH₂CF₃ | 7.6 (d), 7.5 (d), 7.4-7.2 (m), 7.1 (t), 6.3 (s), 3.1 |
| | | | | | | (m), 2.9 (s), 2.8 (s) |
| I-1B.67 | S | H | 6-F | 1 | CH₂CCl₃ | 7.6 (d), 7.4 (d), 7.3-7.2 (m), 7.1 (t), 6.3 (s), 3.4 |
| | | | | | | (s), 3.1-3.0 (m) |
| I-1B.68 | S | H | 7-Cl | 1 | CH₂CF₃ | 69 |
| I-1B.69 | S | H | 7-Cl | 1 | CH₃ | 131 |
| I-1B.70 | S | H | 7-CH₃ | 1 | CH₂CH₃ | 103 |
| I-1B.71 | S | H | 7-Br | 1 | CH₃ | 7.7 (s), 7.5 (m), 7.4 (d), 7.3-7.1 (m), 6.3 (s), 3.0 |
| | | | | | | (s), 2.5 (m), 2.4 (s) |
| I-1B.72 | S | H | 7-Br | 1 | CH₂CH₃ | 7.7 (s), 7.6-7.4 (m), 7.3-7.1 (m), 6.3 (s), 3.0 (s), 2.6 |
| | | | | | | (s), 2.5 (q), 1.1 (t) |
| I-1B.73 | S | H | 7-Cl | 1 | CH₃ | 7.6 (m), 7.4 (d), 7.2 (m), 7.1 (m), 6.3 (s), 3.0 (s), |
| | | | | | | 2.5 (m), 2.3 (s) |
| I-1B.74 | S | H | 7-Cl | 1 | CH₂CH₃ | 7.6 (d), 7.4 (d), 7.3-7.2 (m), 6.3 (s), 3.0 (s), 2.6 |
| | | | | | | (s), 2.5 (q), 1.1 (t) |
| I-1B.75 | S | H | 8-OCF₃ | 1 | CH₂CH₃ | 7.5 (m), 7.4 (d), 7.3-7.1 (m), 6.4 (s), 3.0 (s), 2.6 |
| | | | | | | (s), 2.5 (m), 1.1 (t) |
| I-1B.76 | S | H | 8-Cl | 1 | CH₂CHCH₂ | 7.6 (s), 7.4 (d), 7.2-7.1 (m), 6.3 (s), 5.9 (m), 5.2 |
| | | | | | | (m), 3.0 (s), 2.9 (s), 2.5 (s) |
| I-1B.77 | S | H | 7-CH₃ | 1 | CH₂CF₃ | 122 |
| I-1B.78 | S | H | 7-CH₃ | 1 | CH₂CCl₃ | 179 |
| I-1B.79 | S | H | 7-Br | 1 | CH₂CF₃ | 83 |
| I-1B.80 | S | H | 7-Br | 1 | CH₂CCl₃ | 165 |
| I-1B.81 | S | H | 7-Cl | 1 | CH₂CCl₃ | 186 |
| I-1B.82 | S | H | 7-OCH₃ | 1 | CH₃ | 148 |
| I-1B.83 | S | H | 7-OCH₃ | 1 | CH₂CH₃ | 111 |
| I-1B.84 | S | H | 7-OCH₃ | 1 | CH₂CF₃ | 129 |
| I-1B.85 | CH₂ | H | 8-CH₂CH₃ | 1 | CH₂CH₃ | 7.5 (s), 7.3-7.1 (m), 6.3 (s), 3.7 (m), 3.5 (m), 3.1 |
| | | | | | | (s), 2.9 (s), 2.6 (q), 2.5 (q), 1.2 (t), 1.1 (t) |
| I-1B.86 | CH₂ | H | 8-CH₂CH₃ | 1 | CH₃ | 7.5 (s), 7.2-7.1 (m), 6.3 (s), 3.7 (m), 3.5 (m), 3.2 |
| | | | | | | (s), 3.0 (s), 2.6 (q), 2.3 (s), 1.2 (t) |
| I-1B.87 | S | H | 8-Cl | 2 | CH₃ | 7.4 (m), 7.3-7.2 (m), 7.1 (t), 6.3 (s), 3.4-3.3 (m), 2.8 |
| | | | | | | (s), 2.7 (m), 2.4 (s), 2.0 (s) |
| I-1B.88 | S | H | 8-CH₃ | 2 | CH₃ | 7.5-7.0 (m), 6.2 (s), 3.5-3.3 (m), 2.8-2.6 (m), 2.4 (s), |
| | | | | | | 2.3 (s), 2.0 (m) |

TABLE 2

| | |
|---|---|
| | (I) |
| 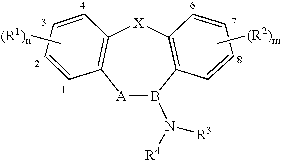 | |

| Compound | A-B | X | (R¹)ₙ | (R²)ₘ | NR³R⁴ | m.p. [° C.] |
|---|---|---|---|---|---|---|
| I-1 | C—C | S | H | 8-Cl | 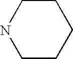 | 131-134 |
| I-2 | C—C | S | H | 8-Cl | 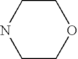 | 140-142 |
| I-3 | C—C | O | H | 8-Cl | 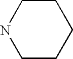 | 85-92 |
| I-4 | C—C | O | H | 8-Cl | 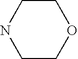 | 87-93 |

Examples of Action Against Plant, Structural and Human Health Pests

The action of the compounds of formula I against pests for example is demonstrated for the compounds of tables 3 and 4 and for a variety of species. Not all compounds of tables 3 and 4 were tested against each of the species.

TABLE 3

| | |
|---|---|
| | (I-1) |
| 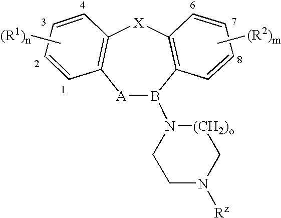 | |

| No. | A-B | X | (R¹)ₙ | (R²)ₘ | o | R^{z} | adduct with |
|---|---|---|---|---|---|---|---|
| I-1.1 | C—C | S | H | 8-SCH₃ | 1 | CH₃ | methane sulfonic acid |
| I-1.2 | C—C | S | H | 8-Cl | 1 | CH₃ | maleic acid |
| I-1.3 | C—C | S | 3-F | 8-CF₃ | 1 | CH₂CH₂OH | dimaleic acid |
| I-1.4 | C—C | S | 2-F | 8-SCH₃ | 1 | CH₂CH₂OH | dimaleic acid |
| I-1.5 | C—C | S(═O) | 3-OCH₃ | 8-Cl | 1 | CH₃ | maleic acid |
| I-1.6 | C—C | S | 3-F | 8-SCH₂CH₃ | 1 | CH₃ | maleic acid |
| I-1.7 | C—C | S | H | 6-F | 1 | CH₂CH₂OH | maleic acid |
| I-1.8 | C—C | S | 2-Cl | 6-F | 1 | CH₃ | maleic acid |
| I-1.9 | C—C | S | 3-F | 8-COCH₃ | 1 | CH₂CH₂OH | dimaleic acid |
| I-1.10 | C—C | S | H | 8-Cl | 1 | CH₂CH₃ | — |
| I-1.11 | C—C | S | H | 8-Cl | 1 | (CH₂)₃CH₃ | — |
| I-1.12 | C—C | S | H | 8-Cl | 1 | CH(CH₃)₂ | — |
| I-1.13 | C—C | S | H | 8-Cl | 1 | C₆H₁₁ | — |
| I-1.14 | C—C | S | 3-Cl | 8-Cl | 1 | CH₃ | — |
| I-1.15 | C—C | S | 2-Cl | 8-Cl | 1 | CH₃ | — |
| I-1.16 | C—C | S | 3-F | 8-SCH₂CH₃ | 1 | CH₂CH₃ | — |
| I-1.17 | C—C | S | H | 6-Cl | 1 | CH₃ | — |
| I-1.18 | C—C | S | H | 8-NH₂ | 1 | CH₃ | — |
| I-1.19 | C—C | 0 | H | 8-Cl | 1 | CH₃ | — |
| I-1.20 | C—C | 0 | H | 8-Cl | 1 | C₆H₁₁ | — |
| I-1.21 | C—C | 0 | H | 8-Cl | 1 | C₆H₅ | — |
| I-1.22 | C—C | CH₂ | H | 8-Cl | 1 | CH₃ | — |
| I-1.23 | C—C | S | H | H | 1 | CH₂CH₃ | — |
| I-1.24 | C—C | S | 3-Cl | H | 1 | CH₂CH₃ | — |
| I-1.25 | C—C | S | 3-F | H | 1 | CH₂CH₃ | — |
| I-1.26 | C—C | S | 3-F | H | 1 | CH₃ | — |
| I-1.27 | C—C | S | 4-F | 8-Cl | 1 | CH₂CH₃ | — |
| I-1.28 | C—C | S | 3-F | H | 1 | CH₃ | — |
| I-1.29 | C—C | S | H | 8-CN | 1 | CH₃ | — |
| I-1.30 | C—C | S | H | 7-Cl | 1 | CH₃ | — |
| I-1.31 | C—C | S | H | 8-F | 1 | CH₃ | — |
| I-1.32 | C—C | S | H | 8-I | 1 | CH₃ | — |
| I-1.33 | C—C | S | H | 8-C₆H₅ | 1 | CH₃ | — |
| I-1.34 | C—C | S | 3-F | 8-Cl | 1 | CH₂CH₃ | — |
| I-1.35 | C—C | S | H | 8-Cl | 1 | CH₂CH₃ | — |
| I-1.36 | C═C | S | 3-F | 8-CH₃ | 1 | CH₂CH₃ | — |
| I-1.37 | C—C | S | 3-Cl | H | 1 | CH₃ | maleic acid |
| I-1.38 | C—C | O | H | 8-Cl | 1 | C(═O)OCH₂CH₃ | — |
| I-1.39 | C—C | S | H | 8-Cl | 1 | (CH₂)20CH₃ | — |
| I-1.40 | C—C | S | 4-Cl | 8-Cl | 1 | CH₃ | — |
| I-1.41 | C—C | S | 3-F | 8-SCH₂CH₃ | 1 | CH₃ | — |
| I-1.42 | C—C | S | 3-F | 8-CH₃ | 1 | CH₂CH₃ | — |
| I-1.43 | C═C | S | 4-F | 8-Cl | 1 | CH₃ | — |
| I-1.44 | C═C | S | 4-Cl | H | 1 | CH₂CH₃ | — |
| I-1.45 | C—C | S | H | 8-CCCH₃ | 1 | CH₃ | — |
| I-1.46 | C—C | S | H | H | 1 | CH₃ | — |
| I-1.47 | C—C | O | H | 8-Cl | 1 | C₆H₁₁ | — |

TABLE 4

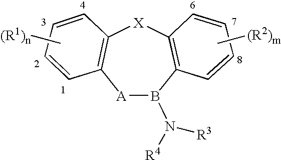

| No. | A-B | X | (R¹)ₙ | (R²)ₘ | NR³R⁴ | adduct with |
|---|---|---|---|---|---|---|
| I.1 | C—C | S | 2-Cl | H | NH₂ | maleic acid |
| I.2 | C—C | S(═O) | 2-Cl | H | HN(CH₂)₂NH₂ | dimaleic acid |
| I.3 | C—C | S | H | 8-Cl | HNC(═O)NHC(CH₃)₃ | |
| I.4 | C—C | S | H | 8-Cl | 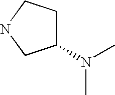 | |
| I.5 | C—C | S | H | 8-Cl | 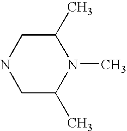 | |
| I.6 | C—C | O | H | 8-Cl | 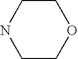 | |
| I.7 | C—C | S | H | 8-SCH₃ | 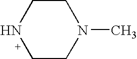 | methane sulfonic acid |
| I.8 | C—C | S | H | H | NC(═S)NCH₂CH₂OH | |

The action of the compounds of the formula I against pests was demonstrated by the following experiments:

Nematicidal Evaluation

Test compounds were prepared and formulated into aqueous formulations using acetone. The formulations were tested using root knot nematode (2nd instar) and soybean cyst nematode (2nd instar) as target species.

Test Procedures for Root-Knot Nematode (Meloidogyne Incognita):

Tomato plants (var. Bonny Best) were grown in the greenhouse in plastic tubs (4 to 6 plants per tub). The plants and soil (a 50:50 mixture of sand and “New Egypt” sandy loam) were infested with M. incognita J2 (to establish the “in-house” colony, M. incognita J2 were initially acquired from Auburn University). The plants were kept pruned and were used on an “as needed” basis. The tomato plants were kept in the cylinder containing hydroponic solution and aerated until the nematodes were no longer present in the solution (usually about 60 days). The cultures were chekked daily by eluting a small volume (approximately 20 ml) from the bottom of a funnel attached to the cylinder into a small crystallizing dish and observed using a binocular dissecting scope. If needed for testing, the nematodes were cleaned and concentrated by pouring the culture solution through a sieve for cleaning and a sieve for concentrating. The nematodes were then resuspended in water to a concentration of approximately 20 to 50 nematodes per 50 μl. These were counted by putting 25 μl of the nematode solution into a well of an unused well of an assay plate. The total was then multiplied by 2 for a final total of nematodes per 50 μl of solution. To microtiter plates containing about 1.0 mg of compound, 80:20 acetone was added to each well and the solution was mixed to obtain the desired compound concentration. The nematode solution was added to each plate. The plates were then sealed and they were placed in an incubator at 27° C. and 50% (+/−2%) relative humidity. After 72 hours, the population mortality was read, whereby immobility of nematodes was regarded as mortality.

In this test, compounds I-1.4, I-1.5, I-1.6, I-1.7, I-1.8, I-1.9, I-1.10, I-1.17, I-1.30, I-1.31, I-1.32, I-1.34, I.1, I.2, and I.6 at 500 ppm showed over 95% mortality compared to untreated controls.

Test Procedures for Soybean Cyst Nematode (Heterodera Glycine):

The soybean bean cyst nematode culture was maintained in a greenhouse and soybean eggs and J2 larvae were obtained for testing by dislodging soybean cysts from the roots with a sieve. The cysts were broken to release the eggs and the eggs were maintained in water. The eggs hatched after 5-7 days at 28° C. To microtiter plates containing about 150 μg of compound, 80:20 acetone was added to each well and the solution was mixed to obtain the desired compound concentration. The nematode solution was added to the plate. The plates were then sealed and placed in an incubator at 27° C. and 50% (+/−2%) relative humidity. After 72 hours, the population mortality was read, whereby immobility of nematodes was regarded as mortality.

In this test, compounds I-1.4, I-1.8, I-1.29, I-1.33, I.2, I.3, and I.8 at 100 ppm showed over 95% mortality compared to untreated controls.

Activity Against Insects and Arachnids

Southern Armyworm (Spodoptera Eridania), 2nd Instar Larvae

The active compounds were formulated for testing the activity against insects and arachnids as a 10.000 ppm solution in a mixture of 35% acetone and water, which was diluted with water, if needed.

A Sieva lima bean leaf expanded to 7-8 cm in length is dipped in the test solution with agitation for 3 seconds and allowed to dry in a hood. The leaf is then placed in a 100×10 mm petri dish containing a damp filter paper on the bottom and ten 2nd instar caterpillars. At 5 days, observations are made of mortality, reduced feeding, or any interference with normal molting.

In this test, compounds I-1.10, I-1.12, I-1.14, I-1.15, I-1.17, I-1.25, I-1.29, I-1.30, I-1.32, I-1.33, I-1.35, I-1.38, I-1.40, I-1.41, I.1, I.2 and I.3 at 1500 ppm showed over 50% mortality in comparison with untreated controls.

Cotton Aphid (Aphis Gossypii)

The active compounds were formulated in 50:50 acetone:water and 100 ppm Kinetic® surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds I-1.1, I-1.2, I-1.3, I-1.6, I-1.10, I-1.11, I-1.12, I-1.13, I-1.14, I-1.16, I-1.17, I-1.19, I-1.20, I-1.21, I-1.22, I-1.25, I-1.29, I-1.32, I-1.33, I-1.34, I-1.35, I-1.37, I-1.41, I-1.42, I.4, I.5, and I.7 at 300 ppm showed over 90% mortality in comparison with untreated controls.

Green Peach Aphid (Myzus Persicae)

The active compounds were formulated in 50:50 acetone:water and 100 ppm Kinetic® surfactant.

Pepper plants in the 2nd leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

Bean Aphid (Aphis Fabae)

The active compounds were formulated in 50:50 acetone:water and 100 ppm Kinetic® surfactant.

Nasturtium plants grown in Metro mix in the 1St leaf-pair stage (variety ‘Mixed Jewle’) were infested with approximately 2-30 laboratory-reared aphids by placing infested cut plants on top of the test plants. The cut plants were removed after 24 hr. Each plant was dipped into the test solution to provide complete coverage of the foliage, stem, protruding seed surface and surrounding cube surface and allowed to dry in the fume hood. The treated plants were kept at about 25° C. with continuous fluorescent light. Aphid mortality was determined after 3 days.

Silverleaf Whitefly (Bemisia Argentifolii)

The active compounds were formulated in 50:50 acetone:water and 100 ppm Kinetic® surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were colleted using an aspirator and an 0.6 cm, non-toxic Tygon® tubing (R-3603) connected to a barrier pipette tip. The tip, containing the collected insects, was then gently inserted into the soil containing the treated plant, allowing insects to crawl out of the tip to reach the foliage for feeding. The cups were covered with a re-usable screened lid (150 micron mesh polyester screen PeCap from Tetko Inc).

Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.

In this test, compounds I-1.1, I-1.2, I-1.6, I-1.11, I-1.12, I-1.16, I-1.25, I-1.32, I-1.33, I-1.34, I-1.35, I-1.36, I-1.37, I-1.41, I-1.42, and 1.7 at 300 ppm showed over 90% mortality compared to untreated controls.

2-Spotted Spider Mite (Tetranychus Urticae, OP-Resistant Strain)

The active compounds were formulated in 50:50 acetone:water and 100 ppm Kinetic® surfactant.

Sieva lima bean plants with primary leaves expanded to 7-12 cm were infested by placing on each a small piece from an infested leaf (with about 100 mites) taken from the main colony. This was done at about 2 hours before treatment to allow the mites to move over to the test plant to lay eggs. The piece of leaf used to transfer the mites was removed. The newly-infested plants were dipped in the test solution and allowed to dry. The test plants were kept under fluorescent light (24 hour photoperiod) at about 25° C. and 20-40% relative humidity. After 5 days, one leaf was removed and mortality counts were made.

In this test, compounds I-1.28, I-1.43, and I-1.44 at 300 ppm showed over 50% mortality compared to untreated controls.

Yellowfever Mosquitos (Aedes Aegypti)

The test compound (1 Vol % in acetone) was applied to water in glass dishes containing 4th instar aedes aegypti. The test dishes were maintained at about 25° C. and observed daily for mortality. Each test weas replicated in 3 test dishes.

In this test, compound I-1.41 at 100 ppm after 6 days showed over 90% mortality compared to untreated controls.

Eastern Subterranean Termites (Reticulitérmes flávipes)

Toxicant treatments (1.0% test compound w/w) were applied to 4.25 cm (diam.) filter papers (VWR #413, qualitative) in acetone solution. Treatment levels (% test compound) were calculated on basis of a mean weight per filter paper of 106.5 mg. Treatment solutions were adjusted to provide the quantity of toxicant (mg) required per paper in 213 ml of acetone (volume required for saturation of paper). Acetone only was applied for untreated controls. Treated papers were vented to evaporate the acetone, moistened with 0.25 ml water, and enclosed in 50×9 mm Petri dishes with tight-fit lids (3-mm hole in side of each dish for termite entry).

Termite bioassays were conducted in 100×15 mm Petri dishes with 10 g fine sand spread in a thin layer over the bottom of each dish. An additional 2.5 g sand was piled against the side of each dish. The sand was moistened with 2.8 ml water applied to the piled sand. Water was added to dishes as needed over the course of the bioassays to maintain high moisture content. Bioassays were done with one treated filter (inside enclosure) and 30 termite workers per test dish. Each treatment level was replicated in 2 test dishes. Test dishes were maintained at about 25° C. and 85% humidity for 12 days and observed daily for mortality.

In this test, compound I-1.41 at 1% (weight per weight filter paper) after 12 days showed 100% mortality, whereas untreated controls showed 8% mortality.

Orchid Thrips (Dichromothrips Corbetti)

Dichromothrips corbetti adults used for bioassay were obtained from a colony maintained continuously under laboratory conditions. For testing purposes, the test compound was diluted to a concentration of 500 ppm (wt compound: vol diluent) in a 1:1 mixture of acetone:water, plus 0.01% Kinetic® surfactant. Thrips potency of each compound was evaluated by using a floral-immersion technique. Plastic petri dishes were used as test arenas. All petals of individual, intact orchid flowers were dipped into treatment solution for approximately 3 seconds and allowed to dry for 2 hours. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, compounds I-1.10 and I-1.19 showed over 50% mortality compared to untreated controls.

1. A method for controlling insects, arachnids or nematodes comprising contacting an insect, arachnid or nematode or their food supply, habitat or breeding grounds with a pesticidally effective amount of a compound of formula I 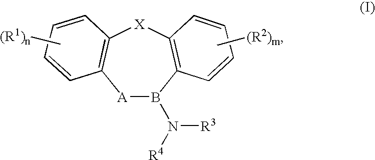
wherein
A-B denotes C—C or C═C;
X is sulfur, oxygen, sulfinyl (S═O), sulfonyl (SO₂), NR^{a}, or CR^{b}R^{c};
R^{a} hydrogen, C₁-C₆-alkyl, C₂-C₆-alkenyl, or C₂-C₆-alkynyl wherein the carbon atoms in these groups may be substituted by 1 to 3 groups R#
R^{#} halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, or C₁-C₆-alkylthio;
phenyl or benzyl, each unsubstituted or substituted with any combination of 1 to 5 halogen, 1 to 3 C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
R^{b}, R^{c} are each independently hydrogen, hydroxy, amino, C₁-C₆-alkyl, C₂-C₆-alkenyl, C₂-C₆-alkinyl, C₁-C₆-hydroxyalkyl, C₁-C₆-alkylamino, di(C₁-C₆-alkyl)amino, wherein the carbon atoms in these groups may be substituted by 1 to 3 groups R#, or
phenyl, unsubstituted or substituted with any combination of 1 to 5 halogen, 1 to 3 C₁-C₆-alkyl, C₁-C₆-haloalkyl, C₁-C₆-alkoxy or C₁-C₆-haloalkoxy groups, or
CR^{b}R^{c} represents C═O or C═CR^{j}R^{k}, wherein R^{j} and R^{k} each independently are hydrogen, halogen, cyano, C₁-C₆-alkyl, C₁-C₆-haloalkyl, C₃-C₆-cycloalkyl;
R¹, R² are each independently hydrogen, halogen, hydroxy, mercapto, amino, cyano, nitro,
C₁-C₆-alkyl, C₁-C₆-alkoxy, C₁-C₆-alkylamino, di(C₁-C₆-alkyl)amino, C₁-C₆-alkylthio, C₂-C₆-alkenyl, C₂-C₆-alkenyloxy, C₂-C₆-alkenylamino, C₂-C₆-alkenylthio, C₂-C₆-alkynyl, C₂-C₆-alkynyloxy, C₂-C₆-alkynylamino, C₂-C₆-alkynylthio, C₁-C₆-alkylsulfonyl, C₁-C₆-alkylsulfoxyl, C₂-C₆-alkenylsulfonyl, C₂-C₆-alkynylsulfoxyl, formyl, C₁-C₆-alkylcarbonyl, hydroxycarbonyl, C₁-C₆-alkoxycarbonyl, carbonyloxy, C₁-C₆-alkylcarbonyloxy, phenyloxy, C(O)NR^{d}R^{e}, (SO₂)ₙNR^{d}R^{e}, wherein the carbon atoms in the aliphatic and aromatic groups may be substituted by 1 to 3 groups R^{#} wherein R^{d} and R^{e} are each independently groups as listed for R^{a}; or
C(═NOR^{f})-Γₚ-R^{f}′, wherein R^{f}′ and R^{f} are each independently hydrogen or C₁-C₆-alkyl, F is oxygen, sulfur or NR^{f} and p is 0 or 1; or
a mono- or bicyclic 5- to 10-membered aromatic ringsystem which may contain 1 to 4 heteroatoms selected from oxygen, sulfur and nitrogen and which is unfused or fused to the aromatic group to which it is bonded and which, when unfused, is bonded directly or through an oxygen, sulfur, C₁-C₆-alkyl, or C₁-C₆-alkoxy linkage, and which is unsubstituted or substituted with any combination of 1 to 5 groups R^{#}; or
C₃-C₁₂-cycloalkyl, which is bonded directly or through an oxygen, sulfur or C₁-C₆-alkyl linkage, and which is unsubstituted or substituted with any combination of 1 to 5 groups R^{#};
R³, R⁴ are each independently hydrogen, C₁-C₆-alkyl, C₁-C₆-haloalkyl, C₁-C₆-alkylamino, C₁-C₆-alkoxy, C₃-C₆-cycloalkyl, wherein the carbon atoms in these aliphatic groups may be substituted by 1 to 3 groups R^{#}, or C(O)R^{g}, C(O)NR^{h}Rⁱ C(S)NR^{h}Rⁱ,
R^{g} hydrogen, C₁-C₆-alkyl, C₁-C₆-alkoxy, or
phenyl or benzyl, each unsubstituted or substituted with any combination of 1 to 5 halogen, 1 to 3 C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
R^{h}, Rⁱ are each independently groups as listed for R^{a};
or R³ and R⁴ together with the nitrogen atom to which they are attached form a saturated or partially saturated mono- or bicyclic 5- to 10-membered ringsystem containing 1 to 3 heteroatoms selected from nitrogen and oxygen or 5-membered hetaryl containing 1 to 4 nitrogen atoms, wherein the carbon and/or nitrogen atoms in the saturated, partially saturated or aromatic rings are unsubstituted or substituted with any combination of 1 to 4 groups selected from amino, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-alkylamino, di(C₁-C₆-alkyl)amino, C₂-C₆-alkenylamino, C₂-C₆-alkynylamino, C₁-C₆-hydroxyalkyl, hydroxycarbonyl-C₁-C₄-alkyl, C₁-C₆-alkoxycarbonyl-C₁-C₄-alkyl, formyl-C₁-C₄-alkyl, formyl-C₁-C₄-alkoxy, C₁-C₆-alkylcarbonyl-C₁-C₄-alkoxy, C₃-C₆-cycloalkyl, which is bonded directly or via an oxygen, sulfur or C₁-C₆-alkyl linkage, and C₅-C₈-cycloalkenyl, wherein the carbon atoms in these aliphatic groups can be substituted by 1 to 4 groups selected from halogen, cyano, hydroxy and nitro, and wherein the nitrogen atoms which form part of the saturated rings can be present as [N⁺—O⁻] (aminoxide);
m is 0, 1, 2, 3 or 4;
n is 0, 1, 2, 3 or 4;
or the enantiomers or diastereomers, salts or esters thereof.

2. A method for controlling insects, arachnids or nematodes comprising contacting an insect, arachnid or nematode or their food supply, habitat or breeding grounds with a pesticidally effective amount of a composition comprising a compound of formula I as defined in claim 1 and an agronomically acceptable carrier.

3. The method according to claim 2, wherein the compositions are formulated into dusting powders or granules, dispersible powders, granules or grains, aqueous dispersions, suspensions, pastes, or emulsions.

4. A method for protecting growing plants from attack or infestation by insects, arachnids or nematodes comprising contacting a plant, or soil or water in which the plant is growing, with a pesticidally effective amount of a compound of formula I as defined in claim 1.

5. A method according to claim 4, wherein the compound of formula I is applied at a rate of about 50 g/ha to about 500 g/ha.

6. The method according to claim 1 or 2, wherein the pests are selected from insects and nematodes.

7. The method according to claim 4 or 5, wherein the pests are selected from insects and nematodes.

8. A method according to claims 1, 2, 4 or 5, wherein the pests are selected from the orders Homoptera, Lepidoptera, Diptera, Thysanoptera, and Nematoda.

9. Compounds of formula I-3 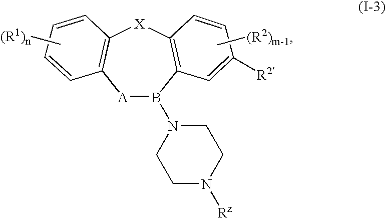
wherein
A-B denotes C—C or C═C;
X is oxygen, sulfur, amino, methylamino or methylene;
R¹ is hydrogen, hydroxy, mercapto, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxycarbonyl, aminosulfonyl (NH₂SO₂), C₁-C₆-alkylaminosulfonyl or di(C₁-C₆-alkyl)aminosulfonyl;
R² is hydrogen, hydroxy, mercapto, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₂-C₆-alkenyl or C₂-C₆-alkynyl;
R²′ is bromo, C₁-C₆-alkyl or C₁-C₆-thioalkyl;
R^{z} is C₂-C₆-alkyl, C₂-C₆-haloalkyl, C₂-C₆-alkenyl, C₂-C₆-alkynyl or C₃-C₁₀-cycloalkyl;
n is 1 or 2;
m is 1 or 2;
or the enantiomers, diastereomers, salts or esters thereof,
with the proviso that
if X is sulfur, A-B denotes C—C, (R¹)ₙ is 3-fluoro, m is zero and R²′, is isopropyl, R^{z} is not ethyl or isopropyl;
if X is oxygen, A-B denotes C—C, n and m are zero and R^{z} is allyl, R², is not ethyl;
if X is oxygen, A-B denotes C—C, n and m are zero and R^{z} is ethyl, R²′ is not methyl.

10. A process for the preparation of compounds of formula I-3 according to claim 9, wherein X is sulfur and A-B denotes C—C, by reduction of compunds of formula I-3, wherein X is sulfur and A-B denotes C═C, which comprises NaBH₃CN as the reduction agent.
